(19) United States
(12) Patent Application Publication
(10) Pub. No.: US 2016/0039842 A1
(43) Pub. Date: Feb. 11, 2016

(54) OXAZOLO[5,4-C]QUINOLIN-2-ONE COMPOUNDS AS BROMODOMAIN INHIBITORS

(71) Applicant: EPIGENETIX, INC., Delray Beach, FL (US)

(72) Inventors: Jeffrey S. ALBERT, Dollard-des-Ormeaux (CA); Shawn JOHNSTONE, Vaudreuil-Dorion (CA); Paul JONES, Verdun (CA)

(21) Appl. No.: 14/776,662

(22) PCT Filed: Mar. 13, 2014
(86) PCT No.: PCT/US14/26837

§ 371 Date: Sep. 14, 2015

Related U.S. Application Data
(60) Provisional application No. 61/789,639, filed on Mar. 15, 2013.

(51) Int. Cl. C07D 498/04

(52) U.S. Cl. CPC C07D 498/04

(57) ABSTRACT

The present invention relates to compounds useful as bromodomain inhibitors. The invention also provides pharmaceutically acceptable compositions comprising compounds of the present invention and methods of using said compounds and compositions in the treatment of various diseases and disorders.

### RELATED APPLICATIONS

This application claims the benefit of U.S. provisional patent application No. 61/789,639 entitled “OXAZOLO[5,4-c]QUINOLIN-2-ONE COMPOUNDS AS BROMODOMAIN INHIBITORS” filed on Mar. 15, 2013, which is herein incorporated by reference in its entirety.

### FIELD OF THE INVENTION

The present invention relates to compounds useful as bromodomain inhibitors.

### BACKGROUND

Bromodomains are found in a variety of mammalian DNA-binding proteins. The bromodomain, which is the conserved structural module in chromatin-associated proteins and histone acetyltranferases, is known to recognize acetyl-lysine residues on proteins. Bromodomain inhibitors are believed to be useful in the treatment of a variety of diseases or conditions, such as cancer as well as chronic autoimmune and inflammatory conditions.

### SUMMARY OF THE INVENTION

Described herein are compounds of Formula (I) useful as bromodomain inhibitors.

In one aspect provided herein is a method for inhibiting activity of a bromodomain-containing protein, or a mutant thereof, in a biological sample comprising the step of contacting said biological sample with a compound of the Formula (I).

In another aspect provided herein is a method for inhibiting activity of a bromodomain-containing protein, or a mutant thereof, activity in a patient comprising the step of administering to said patient a compound of Formula (I).

In another aspect provided herein is a method for treating a bromodomain-containing protein-mediated disorder in a patient in need thereof, comprising the step of administering to said patient a compound of Formula (I).

In another aspect provided herein are compounds, and pharmaceutically acceptable compositions thereof, useful for treating a variety of diseases, disorders or conditions associated with abnormal cellular responses triggered by events mediated by bromodomain-containing proteins. Such diseases, disorders, or conditions include those described herein.

In another aspect provided herein are compounds that are also useful for the study of bromodomain-containing proteins in biological and pathological phenomena, the study of intracellular signal transduction pathways mediated by bromodomain-containing proteins, and the comparative evaluation of new inhibitors of bromodomain-containing proteins.

In another aspect provided herein are compounds of Formula (I):

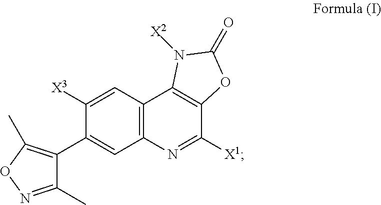

or a pharmaceutically acceptable salt thereof,
wherein:
X¹ is H, —C(O)NR¹R², —C(O)—R¹, —C(O)OR¹, cycloalkyl, heterocycloalkyl, aryl, heteroaryl, —CH₂OR¹, —CH₂R¹, or —C≡N;
X² is H, optionally substituted alkyl, optionally substituted alkenyl, optionally substituted cycloalkyl, optionally substituted aryl, optionally substituted heterocycloalkyl, optionally substituted heteroaryl, optionally substituted —CH₂-cycloalkyl, optionally substituted —CH₂-aryl, optionally substituted —CH₂-heterocycloalkyl, optionally substituted —CH₂-heteroaryl, optionally substituted —CH(C₁-C₆-alkyl)-alkyl, optionally substituted —CH(C₁-C₆-alkyl)-cycloalkyl, optionally substituted —CH(C₁-C₆-alkyl)-aryl, optionally substituted —CH(C₁-C₆-alkyl)-heterocycloalkyl, or optionally substituted —CH(C₁-C₆-alkyl)-heteroaryl;
X³ is —OR³, —C≡N, —CH₂OR³, —NH-alkyl, —N(alkyl)₂, —CH₂N(alkyl)₂, —CH₂NH(alkyl), or halogen, and

wherein R¹, R² and R³ are each independently H, C₁-C₁₂alkyl, cycloalkyl, heterocycloalkyl, aryl, or heteroaryl, optionally substituted with alkyl.

In another aspect is a pharmaceutical composition comprising a compound of Formula (I) with a pharmaceutically acceptable carrier, diluent and excipient.

In another aspect is the use of a compound of Formula (I) for the treatment of a disease or condition for which a bromodomain inhibitor is indicated. In some embodiments is the use of a compound of Formula (I) for the treatment of an auto-immune disorder, an inflammatory disorder, a dermal disorder, or cancer. In another embodiment is the use of a compound of Formula (I) for the treatment of an auto-immune disorder. In another embodiment is the use of a compound of Formula (I) for the treatment of an inflammatory disorder. In some embodiments the inflammatory disorder is rheumatoid arthritis, irritable bowel syndrome or psoriasis. In another embodiment is the use of a compound of Formula (I) for the treatment of cancer. In another embodiment is the use of a compound of Formula (I) for the treatment brain cancer, pancreatic cancer, breast cancer, lung cancer or prostate cancer. In another embodiment is the use of a compound of Formula (I) for the treatment of brain cancer. In some embodiments the brain cancer is glioblastoma multiforme. In another embodiment is the use of a compound of Formula (I) for the treatment of pancreatic cancer. In another embodiment is the use of a compound of Formula (I) for the treatment of breast cancer. In another embodiment is the use of a compound of Formula (I) for the treatment of lung cancer. In another embodiment is the use of a compound of Formula (I) for the treatment of prostate cancer.

In another aspect is the use of a compound of Formula (I) in the manufacture of a medicament for the treatment of a disease or condition for which a bromodomain inhibitor is indicated. In some embodiments is the use of a compound of Formula (I) in the manufacture of a medicament for the treatment of an auto-immune disorder, an inflammatory disorder, a dermal disorder, or cancer. In another embodiment is the use of a compound of Formula (I) in the manufacture of a medicament for the treatment of an auto-immune disorder. In another embodiment is the use of a compound of Formula (I) in the manufacture of a medicament for the treatment of an inflammatory disorder. In some embodiments the inflammatory disorder is rheumatoid arthritis, irritable bowel syndrome or psoriasis. In another embodiment is the use of a compound of Formula (I) in the manufacture of a medicament for the treatment of cancer. In another embodiment is the use of a compound of Formula (I) in the manufacture of a medicament for the treatment brain cancer, pancreatic cancer, breast cancer, lung cancer or prostate cancer. In another embodiment is the use of a compound of Formula (I) in the manufacture of a medicament for the treatment of brain cancer. In some embodiments the brain cancer is glioblastoma multiforme. In another embodiment is the use of a compound of Formula (I) in the manufacture of a medicament for the treatment of pancreatic cancer. In another embodiment is the use of a compound of Formula (I) in the manufacture of a medicament for the treatment of breast cancer. In another embodiment is the use of a compound of Formula (I) in the manufacture of a medicament for the treatment of lung cancer. In another embodiment is the use of a compound of Formula (I) in the manufacture of a medicament for the treatment of prostate cancer.

In another aspect is a method of treating a disease or condition for which a bromodomain inhibitor is indicated in a subject in need thereof which comprises administering a therapeutically effective amount of a compound of Formula (I). In some embodiments is a method of treating an auto-immune disorder, an inflammatory disorder, a dermal disorder, or cancer in a subject in need thereof which comprises administering a therapeutically effective amount of a compound of Formula (I). In another embodiment is a method of treating an auto-immune disorder in a subject in need thereof which comprises administering a therapeutically effective amount of a compound of Formula (I). In another embodiment is a method of treating an inflammatory disorder in a subject in need thereof which comprises administering a therapeutically effective amount of a compound of Formula (I). In some embodiments the inflammatory disorder is rheumatoid arthritis, irritable bowel syndrome or psoriasis. In another embodiment is a method of treating cancer in a subject in need thereof which comprises administering a therapeutically effective amount of a compound of Formula (I). In another embodiment is a method of treating brain cancer, pancreatic cancer, breast cancer, lung cancer or prostate cancer in a subject in need thereof which comprises administering a therapeutically effective amount of a compound of Formula (I). In another embodiment is a method of treating brain cancer in a subject in need thereof which comprises administering a therapeutically effective amount of a compound of Formula (I). In some embodiments the brain cancer is glioblastoma multiforme. In another embodiment is a method of treating pancreatic cancer in a subject in need thereof which comprises administering a therapeutically effective amount of a compound of Formula (I). In another embodiment is a method of treating breast cancer in a subject in need thereof which comprises administering a therapeutically effective amount of a compound of Formula (I). In another embodiment is a method of treating lung cancer in a subject in need thereof which comprises administering a therapeutically effective amount of a compound of Formula (I). In another embodiment is a method of treating prostate cancer in a subject in need thereof which comprises administering a therapeutically effective amount of a compound of Formula (I).

In another aspect is a method for inhibiting a bromodomain which comprising contacting the bromodomain with a compound of Formula (I).

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “bromodomain inhibitor” denotes a compound which inhibits the binding of a bromodomain with its cognate acetylated proteins. In one embodiment the bromodomain inhibitor is a compound which inhibits the binding of a bromodomain to acetylated lysine residues. In a further embodiment the bromodomain inhibitor is a compound which inhibits the binding of a bromodomain to acetylated lysine residues on histones, particularly histones H3 and H4.

In a particular embodiment the bromodomain inhibitor is a compound that inhibits the binding of BET family bromodomains to acetylated lysine residues (hereafter referred to as a “BET family bromodomain inhibitor”). The BET family of bromodomain containing proteins comprises 4 proteins (BRD2, BRD3, BRD4 and BRD-t) which contain tandem bromodomains capable of binding to two acetylated lysine residues in close proximity, increasing the specificity of the interaction.

Definitions of specific functional groups and chemical terms are described in more detail below. For purposes of this invention, the chemical elements are identified in accordance with the Periodic Table of the Elements, CAS version, Handbook of Chemistry and Physics, 75^{th}, Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th}, Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions, p. 268 (E. L. Eliel, Ed., Univ. of Notre Dame Press, Notre Dame, Ind. 1972).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired products of the present invention. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

The number of carbon atoms in a hydrocarbyl substituent can be indicated by the prefix “Cₓ-C_{y},” where x is the minimum and y is the maximum number of carbon atoms in the substituent.

The prefix “halo” indicates that the substituent to which the prefix is attached is substituted with one or more independently selected halogen radicals. For example, “haloalkyl” means an alkyl substituent wherein at least one hydrogen radical is replaced with a halogen radical.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

As used herein a “direct bond” or “covalent bond” refers to a single, double or triple bond. In certain embodiments, a “direct bond” or “covalent bond” refers to a single bond.

The terms “halo” and “halogen” as used herein refer to an atom selected from fluorine (fluoro, —F), chlorine (chloro, —CI), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-6 carbon atoms. In some embodiments, aliphatic groups contain 1-4 carbon atoms, and in yet other embodiments aliphatic groups contain 1-3 carbon atoms. Aliphatic groups include, but are not limited to, alkyl, alkenyl, alkynyl, carbocycle. Suitable aliphatic groups include, but are not limited to, linear or branched, alkyl, alkenyl, and alkynyl groups, and hybrids thereof such as (cycloalkyl)alkyl, (cyclo alkenyl)alkyl or (cycloalkyl)alkenyl.

The term “unsaturated”, as used herein, means that a moiety has one or more units of unsaturation.

As used herein, the following abbreviations may have the following meanings:

| Abbreviation | Term |
|---|---|
| AcOH | Acetic acid |
| Approx. | Approximately |
| Aq. | Aqueous |
| CHCl₃ | Chloroform |
| Cs₂CO₃ | Cesium carbonate |
| d | Day(s) |
| DCM | Dichloromethane |
| DDQ | 2,3-dichloro-5,6-dicyano-p-benzoquinone |
| DIAD | Diisopropylazodicarboxylate |
| DMAP | Dimethylaminopyridine |
| DME | 1,2-dimethoxyethane |
| DMF | N,N-dimethyl formamide |
| EtOAc | Ethyl acetate |
| h | Hour(s) |
| HATU | (dimethylamino)-N,N-dimethyl(3H- |
| | [1,2,3]triazolo[4,5-6]pyridin-3- |
| | yloxy)methaniminium hexafluorophosphate |
| HCl | hydrochloric acid |
| KOtBu | Potassium tert-butoxide |
| LC-MS | Liquid chromatography mass spectrum |
| m | Minute(s) |
| MeCN | Acetonitrile |
| MeOH | Methanol |
| MgSO₄ | Magnesium sulfate |
| MsCl | Methanesulfonyl chloride |
| N₂ | Nitrogen |
| NaBH₄ | Sodium borohydride |
| NaHCO₃ | Sodium bicarbonate |
| NaOH | Sodium hydroxide |
| Na₂SO₄ | Sodium sulfate |
| NMR | Nuclear magnetic resonance |
| Pd(OAc)₂ | Palladium acetate |
| PPh₃ | Triphenylphosphine |
| pTSA | p-Toluenesulfonic acid |
| rt | room temperature |
| TEA | Triethylamine |
| THF | Tetrahydrofuran |
| TFA | trifluoroacetic acid |
| TLC | Thin layer chromatography |

The terms “cycloaliphatic”, “carbocycle”, “cycloalkyl”, “carbocyclyl”, “carbocyclo”, or “carbocyclic”, used alone or as part of a larger moiety, refer to a saturated or partially unsaturated cyclic aliphatic monocyclic or bicyclic ring systems, as described herein, having from 3 to 18 carbon ring atoms, wherein the aliphatic ring system is optionally substituted as defined above and described herein. Cycloaliphatic or cycloalkyl groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons. The terms “cycloaliphatic”, “carbocycle”, “cycloalkyl”, “carbocyclyl”, “carbocyclo”, or “carbocyclic” also include aliphatic rings that are fused to one or more aromatic or nonaromatic rings, where the radical or point of attachment is on an aliphatic ring.

As used herein, the term “cycloalkylene” refers to a bivalent cycloalkyl group. In certain embodiments, a cycloalkylene group is a 1,1-cycloalkylene group (i.e., a spiro-fusedring). Exemplary 1,1-cycloalkylene groups include

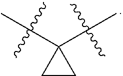 [SMILES: *C1(*)CC1]

In other embodiments, a cycloalkylene group is a 1,2-cycloalkylene group or a 1,3-cycloalkylene group. Exemplary 1,2-cycloalkylene groups include

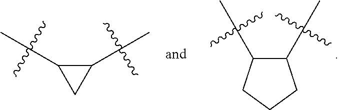 [SMILES: *C1CC1* ; *C1CCCC1*]

The term “alkyl” as used herein, refers to a saturated, straight- or branched-chain hydrocarbon radical typically containing from 1 to 20 carbon atoms. For example, “C₁-C₈ alkyl” contains from one to eight carbon atoms. Examples of alkyl radicals include, but are not limited to, methyl, ethyl, propyl, isopropyl, w-butyl, tert-butyl, neopentyl, n-hexyl, heptyl, octyl radicals and the like.

The term “alkenyl” as used herein, denotes a straight- or branched-chain hydrocarbon radical containing one or more double bonds and typically from 2 to 20 carbon atoms. For example, “C2-C8 alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” as used herein, denotes a straight- or branched-chain hydrocarbon radical containing one or more triple bonds and typically from 2 to 20 carbon atoms. For example, “C2-C8 alkynyl” contains from two to eight carbon atoms.

Representative alkynyl groups include, but are not limited to, for example, ethynyl, 1-propynyl, 1-butynyl, heptynyl, octynyl and the like.

The term “aryl” used alone or as part of a larger moiety as in “aralkyl”, “aralkoxy”, or “aryloxyalkyl”, refers to monocyclic, bicyclic, and tricyclic ring systems having a total of five to 15 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. The term “aralkyl” or “arylalkyl” refers to an alkyl residue attached to an aryl ring.

Examples of aralkyl include, but are not limited to, benzyl, phenethyl and the like. Also included within the scope of the term aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 18 ring atoms, preferably 5, 6, or 9 ring atoms; having 6, 10, or 14 it electrons shared in a cyclic array; and having, in addition to carbon atoms, from one to five heteroatoms. The term “heteroatom” includes but is not limited to nitrogen, oxygen, or sulfur, and includes any oxidized form of nitrogen or sulfur, and any quaternized form of a basic nitrogen. A heteroaryl may be a single ring, or two or more fused rings. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted. Examples include, but are not limited to, pyridinylmethyl, pyrimidinylethyl and the like.

As used herein, the terms “heterocycle”, “heterocycloalkyl”, “heterocyclyl”, “heterocyclic radical”, and “heterocyclic ring” are used interchangeably and refer to a stable 3- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl). Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl and the like.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, 2-azabicyclo[2.2.1]heptanyl, octahydroindolyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond between ring atoms but is not aromatic. The term “partially unsaturated” is intended to encompass rings having multiple sites of unsaturation, but is not intended to include aryl or heteroaryl moieties, as herein defined.

The term “bivalent hydrocarbon” refers to a bivalent saturated or unsaturated hydrocarbon group. Such bivalent hydrocarbon groups include alkylene, alkenylene, and alkynylene groups.

The term “alkylene” refers to a divalent group derived from a straight or branched saturated hydrocarbyl chain typically containing from 1 to 20 carbon atoms, more typically from 1 to 8 carbon atoms. Examples of an “alkylene” include a polymethylene group, i.e., —(CH2)n-, wherein n is a positive integer, preferably from 1 to 6, from 1 to 4, from 1 to 3, from 1 to 2, or from 2 to 3; or —CH₂—, —CH₂CH₂—, —CH₂CH₂CH₂—, —CH₂CH₂CH₂CH₂—, and —CH₂CH(CH₃)CH₂—. A substituted alkylene chain is a polymethylene group in which one or more methylene hydrogen atoms are replaced with a substituent. Suitable substituents include those described below for a substituted aliphatic group.

The term “alkenylene” refers to a divalent unsaturated hydrocarbyl group which may be linear or branched and which has at least one carbon-carbon double bond. An alkenylene group typically contains 2 to 20 carbon atoms, more typically from 2 to 8 carbon atoms. Non-limiting examples of alkenylene groups include —C(H)═C(H)—, —C(H)═C(H)—CH₂—, —C(H)═C(H)—CH₂—CH₂—, —CH₂—C(H)═C(H)—CH₂—, —C(H)═C(H)—CH(CH₃)—, and —CH₂—C(H)═C(H)—CH(CH₂CH₃)—.

The term “alkynylene” refers to a divalent unsaturated hydrocarbon group which may be linear or branched and which has at least one carbon-carbon triple bond.

Representative alkynylene groups include, by way of example, —C≡C—, —C≡C—CH₂—, —C≡C—CH₂—CH₂—, —CH₂—C≡C—CH₂—, —C≡C—CH(CH₃)—, and —CH₂—C≡C—CH(CH₂CH₃)—.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The terms “optionally substituted”, “optionally substituted alkyl,” “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with substituents including, but not limited to:

—F, —CI, —Br, —I,

—OH, protected hydroxy, alkoxy, oxo, thiooxo,

—NO₂, —CN, CF₃, N₃,

—NH₂, protected amino, —NH-alkyl, —NH-alkenyl, —NH-alkynyl, —NH-cycloalkyl, —NH-aryl, —NH-heteroaryl, —NH-heterocyclic, -dialkylamino, -diarylamino,

-diheteroarylamino,

—O-alkyl, —O-alkenyl, —O-alkynyl, —O-cycloalkyl, —O-aryl, —O-heteroaryl,

—O-heterocyclic,

—C(O)-alkyl, —C(O)-alkenyl, —C(O)-alkynyl, —C(O)-cycloalkyl, —C(O)-aryl,

—C(O)-heteroaryl, —C(O)-heterocycloalkyl,

—CONH₂, —CONH— alkyl, —CONH— alkenyl, —CONH-alkynyl, —CONH— cycloalkyl, —CONH-aryl, —CONH-heteroaryl, —CONH-heterocycloalkyl,

—O—CO₂-alkyl, —O—CO₂-alkenyl, —O—CO₂-alkynyl, —O—CO₂-cycloalkyl, —O—CO₂-aryl, —O—CO₂-heteroaryl, —O—CO₂-heterocycloalkyl, —OCONH₂, —OCONH-alkyl, —OCONH-alkenyl, —OCONH-alkynyl, —OCONH-cycloalkyl, —OCONH-aryl, —OCONH-heteroaryl,

—OCONH-heterocycloalkyl,

—NHC(O)-alkyl, —NHC(O)-alkenyl, —NHC(O)— alkynyl, —NHC(O)-cycloalkyl,

—NHC(O)-aryl, —NHC(O)-heteroaryl, —NHC(O)-heterocycloalkyl, —NHCO₂-alkyl,

—NHCO₂-alkenyl, —NHCO₂-alkynyl, —NHCO₂-cycloalkyl, —NHCO₂-aryl,

—NHCO₂-heteroaryl, —NHCO₂-heterocycloalkyl, —NHC(O)NH₂, —NHC(O)NH-alkyl,

—NHC(O)NH-alkenyl, —NHC(O)NH-alkenyl, —NHC(O)NH-cycloalkyl, —NHC(O)NH-aryl, —NHC(O)NH-heteroaryl, —NHC(O)NH-heterocycloalkyl, NHC(S)NH₂, —NHC(S)NH-alkyl, —NHC(S)NH-alkenyl, —NHC(S)NH— alkynyl, —NHC(S)NH-cycloalkyl, —NHC(S)NH-aryl, —NHC(S)NH-heteroaryl, —NHC(S)NH-heterocycloalkyl, —NHC(NH)NH₂,

—NHC(NH)NH-alkyl, —NHC(NH)NH-alkenyl, —NHC(NH)NH-alkenyl,

—NHC(NH)NH-cycloalkyl, —NHC(NH)NH-aryl, —NHC(NH)NH-heteroaryl,

—NHC(NH)NH-heterocycloalkyl, —NHC(NH)-alkyl, —NHC(NH)-alkenyl, —NHC(NH)— alkenyl, —NHC(NH)-cycloalkyl, —NHC(NH)-aryl, —NHC(NH)-heteroaryl,

—NHC(NH)-heterocycloalkyl,

—C(NH)NH-alkyl, —C(NH)NH-alkenyl, —C(NH)NH-alkynyl, —C(NH)NH-cycloalkyl, —C(NH)NH-aryl, —C(NH)NH-heteroaryl, —C(NH)NH-heterocycloalkyl,

—S(O)-alkyl, —S(O)-alkenyl, —S(O)-alkynyl, —S(O)-cycloalkyl, —S(O)-aryl,

—S(O)₂-alkyl, —S(O)₂-alkenyl, —S(O)₂-alkynyl, —S(O)₂-cycloalkyl, —S(O)₂-aryl,

—S(O)-heteroaryl, —S(O)-heterocycloalkyl-SO₂NH₂, —SO₂NH-alkyl, —SO₂NH-alkenyl, —SO₂NH-alkynyl, —SO₂NH-cycloalkyl, —SO₂NH-aryl, —SO₂NH-heteroaryl,

—SO₂NH-heterocycloalkyl,

—NH—SO₂-alkyl, —NH—SO₂-alkenyl, —NH—SO₂-alkynyl, —NH—SO₂-cycloalkyl, —NH—SO₂-aryl, —NH—SO₂-heteroaryl, —NH—SO₂-heterocycloalkyl, —CH₂NH₂, —CH₂—SO₂CH₃,

-alkyl, -alkenyl, -alkynyl, -aryl, -arylalkyl, -heteroaryl, -heteroarylalkyl,

-heterocycloalkyl, -cycloalkyl, -carbocyclic, -heterocyclic, polyalkoxyalkyl, polyalkoxy, -methoxymethoxy, -methoxyethoxy, —SH, —S-alkyl, —S-alkenyl, —S-alkynyl, —S-cycloalkyl, —S-aryl, —S-heteroaryl, —S-heterocycloalkyl, or methylthiomethyl.

In certain embodiments, suitable monovalent substituents on a substitutable carbon atom of an “optionally substituted” group are independently halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})2; —(CH₂)₀₋₄SR^{∘}; —(CH₂)₀₋₄Ph, which may be substituted with R^{∘}; —(CH₂)₀₋₄O(CH₂)₀₋₄Ph which may be substituted with R^{∘};

—CH═CHPh, which may be substituted with R^{∘}; —NO₂; —CN; —N₃; —(CH2))₀₋₄N(R^{∘})₂;

—(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂;

—(CH₂)₀₋₄N(R^{∘}C(O)OR^{∘}; —N(R^{∘})N(R^{∘})C(O)R^{∘}; —N(R^{∘})N(R^{∘})C(O)NR^{∘}₂;

—N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘};

—(CH2)₀₋₄SC(O)R^{∘}; —(CH2)₀₋₄C(O)NR^{∘}₂; —C(S)NR^{∘}₂; —C(S)SR^{∘}; —SC(S)SR^{∘},

—(CH2)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘};

—(CH₂)₀₋₄SSR^{∘}; —(CH₂)₀₋₄S(O)₂R^{∘}; —(CH₂)₀₋₄(O)₂OR^{∘}; —(CH₂)₀₋₄OS(O)₂R^{∘}; —S(O)₂NR^{∘}₂;

—(CH2)₀₋₄S(O)R^{∘}; —N(R^{∘})S(O)₂NR^{∘}₂; —N(R^{∘}S(O)₂R^{∘}; —N(OR^{∘})R^{∘}; —C(NH)NR^{∘}₂; —P(O)₂R^{∘};

—P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3 to 12 membered saturated, partially unsaturated, or aryl mono- or bicyclic ring having 0 to 4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH2)₀₋₂R*, -(haloR*), —(CH2)₀₋₂OH, —(CH2)₀₋₂OR*, —(CH2)₀₋₂CH(OR*)₂, —O(haloR′), —CN, —N₃, —(CH₂)₀₋₂C(O)R*, —(CH₂)₀₋₂C(O)OH, —(CH₂)₀₋₂C(O)OR*, —(CH₂)₀₋₂SR*, —(CH₂)o-₂SH, —(CH₂)₀₋₂NH₂, —(CH₂)₀₋₂NHR*, —(CH₂)₀₋₂NR*₂, —NO₂, —SiR*3, —OSiR*₃, —C(O)SR*—(C₁₋₄ straight or branched alkylene)C(O)OR*, or —SSR*, wherein each R* is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃₀—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR₂)₂₋₃₀—, wherein each independent occurrence of R is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R*, -(haloR*), —OH, —OR*, —O(haloR′), —CN, —C(O)OH, —C(O)OR*, —NH₂, —NHR*, —NR*₂, or —NO₂, wherein each R* is unsubstituted or where preceded by “halo” is substituted only with one or more halogens, and is independently C₁₋₄ aliphatic, —CH2Ph, —O(CH2)₀₋₁Ph, or a 5-6-membered saturated, partially unsaturated, or aryl ring having 0-4 heteroatoms independently selected from nitrogen, oxygen, or sulfur.

As used herein, the term “inhibitor” is defined as a compound that binds to and/or inhibits the target bromodomain-containing protein (such as a BET protein, e.g., BRD2, BRD3, BRD4, and/or BRDT) with measurable affinity. In certain embodiments, an inhibitor has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in activity of at least one bromodomain-containing protein between a sample comprising a provided compound, or composition thereof, and at least one histone methyltransferase, and an equivalent sample comprising at least one bromodomain-containing protein, in the absence of said compound, or composition thereof.

The term “subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts, or salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, or magnesium salts, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds formed by the process of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development”, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology”, John Wiley and Sons, Ltd. (2002).

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

In one aspect provided herein are compounds of Formula (I):

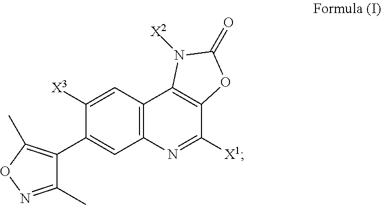

or a pharmaceutically acceptable salt thereof,
wherein:
X¹ is H, —C(O)NR¹R², —C(O)R¹, —C(O)OR¹, cycloalkyl, heterocycloalkyl, aryl, heteroaryl, —CH₂OR¹, —CH₂R¹, or —C≡N;
X² is H, optionally substituted alkyl, optionally substituted alkenyl, optionally substituted cycloalkyl, optionally substituted aryl, optionally substituted heterocycloalkyl, optionally substituted heteroaryl, optionally substituted —CH₂-cyloalkyl, optionally substituted —CH₂-aryl, optionally substituted —CH₂-heterocycloalkyl, optionally substituted —CH₂-heteroaryl, optionally substituted —CH(C₁-C₆-alkyl)-alkyl, optionally substituted —CH(C₁-C₆-alkyl)-cycloalkyl, optionally substituted —CH(C₁-C₆-alkyl)-aryl, optionally substituted —CH(C₁-C₆-alkyl)-heterocycloalkyl, or optionally substituted —CH(C₁-C₆-alkyl)-heteroaryl;
X³ is —OR³, —C≡N, —CH₂OR³, —NH-alkyl, —N(alkyl)₂, —CH₂N(alkyl)₂, —CH₂NH(alkyl), or halogen, and

wherein R¹, R² and R³ are each independently H, C₁-C₁₂alkyl, cycloalkyl, heterocycloalkyl, aryl, or heteroaryl, optionally substituted with alkyl.

In another embodiment is a compound of Formula (I) wherein X³ is —OR³. In another embodiment is a compound of Formula (I) wherein X³ is —OR³ and R³ is H. In another embodiment is a compound of Formula (I) wherein X³ is —OR³ and R³ is C₁-C₁₂alkyl. In another embodiment is a compound of Formula (I) wherein X³ is —OR³ and R³ is C₁-C₆alkyl. In another embodiment is a compound of Formula (I) wherein X³ is —OR³ and R³ is methyl. In another embodiment is a compound of Formula (I) wherein X³ is —OR³ and R³ is ethyl.

In another embodiment is a compound of Formula (I) wherein X¹ is H. In another embodiment is a compound of Formula (I) wherein X¹ is —C(O)NR¹R², —C(O)R¹, —C(O)OR¹, cycloalkyl, heterocycloalkyl, aryl, heteroaryl, —CH₂OR¹, or —CH₂R¹. In another embodiment is a compound of Formula (I) wherein X¹ is —C(O)NR¹R², and R¹ and R² are each independently H or C₁-C₆alkyl. In another embodiment is a compound of Formula (I) wherein X¹ is —C(O)NR¹R², and R¹ is H and R² is C₁-C₆alkyl. In another embodiment is a compound of Formula (I) wherein X¹ is —C(O)NR¹R², and R¹ is H and R² is methyl. In another embodiment is a compound of Formula (I) wherein X¹ is —C(O)NR¹R², and R¹ and R² are each H. In another embodiment is a compound of Formula (I) wherein X¹ is —C(O)NR¹R², and R¹ and R² are each C₁-C₆alkyl. In another embodiment is a compound of Formula (I) wherein X¹ is —C(O)NR¹R², and R¹ and R² are each methyl. In another embodiment is a compound of Formula (I) wherein X¹ is —C(O)R¹, and R¹ is heterocycloalkyl. In another embodiment is a compound of Formula (I) wherein X¹ is —C(O)R¹, and R¹ is morpholinyl. In another embodiment is a compound of Formula (I) wherein X¹ is —C(O)OR¹, and R¹ is H or C₁-C₆alkyl. In another embodiment is a compound of Formula (I) wherein X¹ is —C(O)OR¹, and R¹ is H. In another embodiment is a compound of Formula (I) wherein X¹ is —C(O)OR¹, and R¹ is C₁-C₆alkyl. In another embodiment is a compound of Formula (I) wherein X¹ is —C(O)OR¹, and R¹ is methyl. In another embodiment is a compound of Formula (I) wherein X¹ is —C(O)OR¹, and R¹ is ethyl. In another embodiment is a compound of Formula (I) wherein X¹ is cycloalkyl. In another embodiment is a compound of Formula (I) wherein X¹ is cyclohexyl. In another embodiment is a compound of Formula (I) wherein X¹ is heterocycloalkyl. In another embodiment is a compound of Formula (I) wherein X¹ is piperidinyl. In another embodiment is a compound of Formula (I) wherein X¹ is aryl. In another embodiment is a compound of Formula (I) wherein X¹ is phenyl. In another embodiment is a compound of Formula (I) wherein X¹ is heteroaryl. In another embodiment is a compound of Formula (I) wherein X¹ is imidazolyl. In another embodiment is a compound of Formula (I) wherein X¹ is —CH₂OR¹. In another embodiment is a compound of Formula (I) wherein X¹ is —CH₂OR¹, and R¹ is aryl. In another embodiment is a compound of Formula (I) wherein X¹ is —CH₂OR¹, and R¹ is phenyl. In another embodiment is a compound of Formula (I) wherein X¹ is —CH₂OR¹, and R¹ is C₁-C₆alkyl. In another embodiment is a compound of Formula (I) wherein X¹ is —CH₂OR¹, and R¹ is methyl. In another embodiment is a compound of Formula (I) wherein X¹ is —CH₂R¹. In another embodiment is a compound of Formula (I) wherein X¹ is —CH₂R¹, and R¹ is aryl. In another embodiment is a compound of Formula (I) wherein X¹ is —CH₂R¹, and R¹ is phenyl. In another embodiment is a compound of Formula (I) wherein X¹ is —CH₂R¹, and R¹ is C₁-C₆alkyl.

In another embodiment is a compound of Formula (I) wherein X² is H. In another embodiment is a compound of Formula (I) wherein X² is alkenyl. In another embodiment is a compound of Formula (I) wherein X² is allyl. In another embodiment is a compound of Formula (I) wherein X² is —CH₂-aryl optionally substituted with halogen or methoxy. In another embodiment is a compound of Formula (I) wherein X² is —CH₂-aryl optionally substituted with chloro or methoxy. In another embodiment is a compound of Formula (I) wherein X² is unsubstituted —CH₂-aryl. In another embodiment is a compound of Formula (I) wherein X² is benzyl optionally substituted with chloro or methoxy. In another embodiment is a compound of Formula (I) wherein X² is unsubstituted benzyl. In another embodiment is a compound of Formula (I) wherein X² is —CH₂-heteroaryl optionally substituted with halogen, trifluoromethyl, or methoxy. In another embodiment is a compound of Formula (I) wherein X² is —CH₂-pyridinyl or —CH₂-furanyl. In another embodiment is a compound of Formula (I) wherein X² is unsubstituted —CH₂-pyridinyl. In another embodiment is a compound of Formula (I) wherein X² is —CH₂-heterocycloalkyl. In another embodiment is a compound of Formula (I) wherein X² is —CH₂-piperidinyl or —CH₂-tetrahydropyranyl. In some embodiments is a compound of Formula (I) wherein X² is —CH₂-piperidinyl. In some embodiments is a compound of Formula (I) wherein X² is —CH₂-tetrahydropyranyl. In another embodiment is a compound of Formula (I) wherein X² is —CH₂-cycloalkyl. In another embodiment is a compound of Formula (I) wherein X² is —CH₂-cyclohexyl. In another embodiment is a compound of Formula (I) wherein X² is —CH(C₁-C₆-alkyl)-aryl. In another embodiment is a compound of Formula (I) wherein X² is —CH(CH₃)-phenyl. In another embodiment is a compound of Formula (I) wherein X² is aryl. In another embodiment is a compound of Formula (I) wherein X² is phenyl. In another embodiment is a compound of Formula (I) wherein X² is heteroaryl. In another embodiment is a compound of Formula (I) wherein X² is pyridinyl.

In another embodiment provided herein are compounds of Formula (Ia):

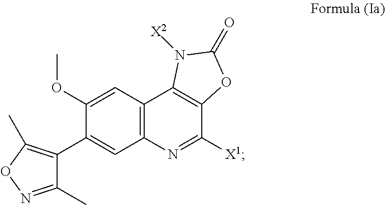

or a pharmaceutically acceptable salt thereof,
wherein:
X¹ is H, —C(O)NR¹R², —C(O)R¹, —C(O)OR¹, cycloalkyl, heterocycloalkyl, aryl, heteroaryl, —CH₂OR¹, —CH₂R¹, or —C≡N;
X² is H, optionally substituted alkyl, optionally substituted alkenyl, optionally substituted cycloalkyl, optionally substituted aryl, optionally substituted heterocycloalkyl, optionally substituted heteroaryl, optionally substituted —CH₂-cycloalkyl, optionally substituted —CH₂-aryl, optionally substituted —CH₂-heterocycloalkyl, optionally substituted —CH₂-heteroaryl, optionally substituted —CH(C₁-C₆-alkyl)-alkyl, optionally substituted —CH(C₁-C₆-alkyl)-cycloalkyl, optionally substituted —CH(C₁-C₆-alkyl)-aryl, optionally substituted —CH(C₁-C₆-alkyl)-heterocycloalkyl, or optionally substituted —CH(C₁-C₆-alkyl)-heteroaryl; and
R¹ and R² are each independently H, C₁-C₁₂alkyl, cycloalkyl, heterocycloalkyl, aryl, or heteroaryl.

In another embodiment is a compound of Formula (Ia) wherein X¹ is H. In another embodiment is a compound of Formula (Ia) wherein X¹ is —C(O)NR¹R², —C(O)R¹, —C(O)OR¹, cycloalkyl, heterocycloalkyl, aryl, heteroaryl, —CH₂OR¹, or —CH₂R¹. In another embodiment is a compound of Formula (Ia) wherein X¹ is —C(O)NR¹R², and R¹ and R² are each independently H or C₁-C₆alkyl. In another embodiment is a compound of Formula (Ia) wherein X¹ is —C(O)NR¹R², and R¹ is H and R² is C₁-C₆alkyl. In another embodiment is a compound of Formula (Ia) wherein X¹ is —C(O)NR¹R², and R¹ is H and R² is methyl. In another embodiment is a compound of Formula (Ia) wherein X ¹ is —C(O)NR¹R², and R¹ and R² are each H. In another embodiment is a compound of Formula (Ia) wherein X¹ is —C(O)NR¹R², and R¹ and R² are each C₁-C₆alkyl. In another embodiment is a compound of Formula (Ia) wherein X¹ is —C(O)NR¹R², and R¹ and R² are each methyl. In another embodiment is a compound of Formula (Ia) wherein X¹ is —C(O)R¹, and R¹ is heterocycloalkyl. In another embodiment is a compound of Formula (Ia) wherein X¹ is —C(O)R¹, and R¹ is morpholinyl. In another embodiment is a compound of Formula (Ia) wherein X¹ is —C(O)OR¹, and R¹ is H or C₁-C₆alkyl. In another embodiment is a compound of Formula (Ia) wherein X¹ is —C(O)OR¹, and R¹ is H. In another embodiment is a compound of Formula (Ia) wherein X¹ is —C(O)OR¹, and R¹ is C₁-C₆alkyl. In another embodiment is a compound of Formula (Ia) wherein X¹ is —C(O)OR¹, and R¹ is methyl. In another embodiment is a compound of Formula (Ia) wherein X¹ is —C(O)OR¹, and R¹ is ethyl. In another embodiment is a compound of Formula (Ia) wherein X¹ is cycloalkyl. In another embodiment is a compound of Formula (Ia) wherein X¹ is cyclohexyl. In another embodiment is a compound of Formula (Ia) wherein X¹ is heterocycloalkyl. In another embodiment is a compound of Formula (Ia) wherein X¹ is piperidinyl. In another embodiment is a compound of Formula (Ia) wherein X¹ is aryl. In another embodiment is a compound of Formula (Ia) wherein X¹ is phenyl. In another embodiment is a compound of Formula (Ia) wherein X¹ is heteroaryl. In another embodiment is a compound of Formula (Ia) wherein X¹ is imidazolyl. In another embodiment is a compound of Formula (Ia) wherein X¹ is —CH₂OR¹. In another embodiment is a compound of Formula (Ia) wherein X¹ is —CH₂OR¹, and R¹ is aryl. In another embodiment is a compound of Formula (Ia) wherein X¹ is —CH₂OR¹, and R¹ is phenyl. In another embodiment is a compound of Formula (Ia) wherein X¹ is —CH₂OR¹, and R¹ is C₁-C₆alkyl. In another embodiment is a compound of Formula (Ia) wherein X¹ is —CH₂OR¹, and R¹ is methyl. In another embodiment is a compound of Formula (Ia) wherein X¹ is —CH₂R¹. In another embodiment is a compound of Formula (Ia) wherein X¹ is —CH₂R¹, and R¹ is aryl. In another embodiment is a compound of Formula (Ia) wherein X¹ is —CH₂R¹, and R¹ is phenyl. In another embodiment is a compound of Formula (Ia) wherein X¹ is —CH₂R¹, and R¹ is C₁-C₆alkyl.

In another embodiment is a compound of Formula (Ia) wherein X² is H. In another embodiment is a compound of Formula (Ia) wherein X² is alkenyl. In another embodiment is a compound of Formula (Ia) wherein X² is allyl. In another embodiment is a compound of Formula (Ia) wherein X² is —CH₂-aryl optionally substituted with halogen or methoxy. In another embodiment is a compound of Formula (Ia) wherein X² is —CH₂-aryl optionally substituted with chloro or methoxy. In another embodiment is a compound of Formula (Ia) wherein X² is unsubstituted —CH₂-aryl. In another embodiment is a compound of Formula (Ia) wherein X² is benzyl optionally substituted with chloro or methoxy. In another embodiment is a compound of Formula (Ia) wherein X² is unsubstituted benzyl. In another embodiment is a compound of Formula (Ia) wherein X² is —CH₂-heteroaryl optionally substituted with halogen, trifluoromethyl, or methoxy. In another embodiment is a compound of Formula (Ia) wherein X² is —CH₂-pyridinyl or —CH₂-furanyl. In another embodiment is a compound of Formula (Ia) wherein X² is unsubstituted —CH₂-pyridinyl. In another embodiment is a compound of Formula (Ia) wherein X² is —CH₂-heterocycloalkyl. In another embodiment is a compound of Formula (Ia) wherein X² is —CH₂-piperidinyl or —CH₂-tetrahydropyranyl. In some embodiments is a compound of Formula (Ia) wherein X² is —CH₂-piperidinyl. In some embodiments is a compound of Formula (Ia) wherein X² is —CH₂-tetrahydropyranyl. In another embodiment is a compound of Formula (Ia) wherein X² is —CH₂-cycloalkyl. In another embodiment is a compound of Formula (Ia) wherein X² is —CH₂-cyclohexyl. In another embodiment is a compound of Formula (Ia) wherein X² is —CH(C₁-C₆-alkyl)-aryl. In another embodiment is a compound of Formula (Ia) wherein X² is —CH(CH₃)-phenyl. In another embodiment is a compound of Formula (Ia) wherein X² is aryl. In another embodiment is a compound of Formula (Ia) wherein X² is phenyl. In another embodiment is a compound of Formula (Ia) wherein X² is heteroaryl. In another embodiment is a compound of Formula (Ia) wherein X² is pyridinyl.

In another is a compound of Formula (Ia) wherein X¹ is H and X² is H, optionally substituted alkyl, optionally substituted alkenyl, optionally substituted cycloalkyl, optionally substituted aryl, optionally substituted heterocycloalkyl, optionally substituted heteroaryl, optionally substituted —CH₂-cycloalkyl, optionally substituted —CH₂-aryl, optionally substituted —CH₂-heterocycloalkyl, optionally substituted —CH₂-heteroaryl, optionally substituted —CH(C₁-C₆-alkyl)-alkyl, optionally substituted —CH(C₁-C₆-alkyl)-cycloalkyl, optionally substituted —CH(C₁-C₆-alkyl)-aryl, optionally substituted —CH(C₁-C₆-alkyl)-heterocycloalkyl, or optionally substituted —CH(C₁-C₆-alkyl)-heteroaryl.

In another is a compound of Formula (Ia) wherein X¹ is H and X² is optionally substituted alkyl. In another is a compound of Formula (Ia) wherein X¹ is H and X² is optionally substituted alkenyl. In another is a compound of Formula (Ia) wherein X¹ is H and X² is optionally substituted cycloalkyl. In another is a compound of Formula (Ia) wherein X¹ is H and X² is optionally substituted aryl. In another is a compound of Formula (Ia) wherein X¹ is H and X² is optionally substituted heterocycloalkyl. In another is a compound of Formula (Ia) wherein X¹ is H and X² is optionally substituted heteroaryl. In another is a compound of Formula (Ia) wherein X¹ is H and X² is optionally substituted —CH₂-cycloalkyl. In another is a compound of Formula (Ia) wherein X¹ is H and X² is optionally substituted —CH₂-aryl. In another is a compound of Formula (Ia) wherein X¹ is H and X² is optionally substituted —CH₂-heterocycloalkyl. In another is a compound of Formula (Ia) wherein X¹ is H and X² is optionally substituted —CH₂-heteroaryl. In another is a compound of Formula (Ia) wherein X¹ is H and X² is optionally substituted —CH(C₁-C₆-alkyl)-alkyl. In another is a compound of Formula (Ia) wherein X¹ is H and X² is optionally substituted —CH(C₁-C₆-alkyl)-heterocycloalkyl. In another is a compound of Formula (Ia) wherein X¹ is H and X² is optionally substituted —CH(C₁-C₆-alkyl)-heteroaryl.

Further described herein are compounds of Formula (Ia) useful as bromodomain inhibitors.

In some embodiments provided herein is a method for inhibiting activity of a bromodomain-containing protein, or a mutant thereof, in a biological sample comprising the step of contacting said biological sample with a compound of the Formula (Ia).

In some embodiments provided herein is a method for inhibiting activity of a bromodomain-containing protein, or a mutant thereof, activity in a patient comprising the step of administering to said patient a compound of Formula (Ia).

In some embodiments provided herein is a method for treating a bromodomain-containing protein-mediated disorder in a patient in need thereof, comprising the step of administering to said patient a compound of Formula (Ia).

In some embodiments is a pharmaceutical composition comprising a compound of Formula (Ia) with a pharmaceutically acceptable carrier, diluent and excipient.

In some embodiments is the use of a compound of Formula (Ia) for the treatment of a disease or condition for which a bromodomain inhibitor is indicated. In some embodiments is the use of a compound of Formula (Ia) for the treatment of an auto-immune disorder, an inflammatory disorder, a dermal disorder, or cancer. In another embodiment is the use of a compound of Formula (Ia) for the treatment of an auto-immune disorder. In another embodiment is the use of a compound of Formula (Ia) for the treatment of an inflammatory disorder. In some embodiments the inflammatory disorder is rheumatoid arthritis, irritable bowel syndrome or psoriasis. In another embodiment is the use of a compound of Formula (Ia) for the treatment of cancer. In another embodiment is the use of a compound of Formula (Ia) for the treatment brain cancer, pancreatic cancer, breast cancer, lung cancer or prostate cancer. In another embodiment is the use of a compound of Formula (Ia) for the treatment of brain cancer. In some embodiments the brain cancer is glioblastoma multiforme. In another embodiment is the use of a compound of Formula (Ia) for the treatment of pancreatic cancer. In another embodiment is the use of a compound of Formula (Ia) for the treatment of breast cancer. In another embodiment is the use of a compound of Formula (Ia) for the treatment of lung cancer. In another embodiment is the use of a compound of Formula (Ia) for the treatment of prostate cancer.

In some embodiments is the use of a compound of Formula (Ia) in the manufacture of a medicament for the treatment of a disease or condition for which a bromodomain inhibitor is indicated. In some embodiments is the use of a compound of Formula (Ia) in the manufacture of a medicament for the treatment of an auto-immune disorder, an inflammatory disorder, a dermal disorder, or cancer. In another embodiment is the use of a compound of Formula (Ia) in the manufacture of a medicament for the treatment of an auto-immune disorder. In another embodiment is the use of a compound of Formula (Ia) in the manufacture of a medicament for the treatment of an inflammatory disorder. In some embodiments the inflammatory disorder is rheumatoid arthritis, irritable bowel syndrome or psoriasis. In another embodiment is the use of a compound of Formula (Ia) in the manufacture of a medicament for the treatment of cancer. In another embodiment is the use of a compound of Formula (Ia) in the manufacture of a medicament for the treatment brain cancer, pancreatic cancer, breast cancer, lung cancer or prostate cancer. In another embodiment is the use of a compound of Formula (Ia) in the manufacture of a medicament for the treatment of brain cancer. In some embodiments the brain cancer is glioblastoma multiforme. In another embodiment is the use of a compound of Formula (Ia) in the manufacture of a medicament for the treatment of pancreatic cancer. In another embodiment is the use of a compound of Formula (Ia) in the manufacture of a medicament for the treatment of breast cancer. In another embodiment is the use of a compound of Formula (Ia) in the manufacture of a medicament for the treatment of lung cancer. In another embodiment is the use of a compound of Formula (Ia) in the manufacture of a medicament for the treatment of prostate cancer.

In some embodiments is a method of treating a disease or condition for which a bromodomain inhibitor is indicated in a subject in need thereof which comprises administering a therapeutically effective amount of a compound of Formula (Ia). In some embodiments is a method of treating an auto-immune disorder, an inflammatory disorder, a dermal disorder, or cancer in a subject in need thereof which comprises administering a therapeutically effective amount of a compound of Formula (Ia). In another embodiment is a method of treating an auto-immune disorder in a subject in need thereof which comprises administering a therapeutically effective amount of a compound of Formula (Ia). In another embodiment is a method of treating an inflammatory disorder in a subject in need thereof which comprises administering a therapeutically effective amount of a compound of Formula (Ia). In some embodiments the inflammatory disorder is rheumatoid arthritis, irritable bowel syndrome or psoriasis. In another embodiment is a method of treating cancer in a subject in need thereof which comprises administering a therapeutically effective amount of a compound of Formula (Ia). In another embodiment is a method of treating brain cancer, pancreatic cancer, breast cancer, lung cancer or prostate cancer in a subject in need thereof which comprises administering a therapeutically effective amount of a compound of Formula (Ia). In another embodiment is a method of treating brain cancer in a subject in need thereof which comprises administering a therapeutically effective amount of a compound of Formula (Ia). In some embodiments the brain cancer is glioblastoma multiforme. In another embodiment is a method of treating pancreatic cancer in a subject in need thereof which comprises administering a therapeutically effective amount of a compound of Formula (Ia). In another embodiment is a method of treating breast cancer in a subject in need thereof which comprises administering a therapeutically effective amount of a compound of Formula (Ia). In another embodiment is a method of treating lung cancer in a subject in need thereof which comprises administering a therapeutically effective amount of a compound of Formula (Ia). In another embodiment is a method of treating prostate cancer in a subject in need thereof which comprises administering a therapeutically effective amount of a compound of Formula (Ia).

In another embodiment is a method for inhibiting a bromodomain which comprising contacting the bromodomain with a compound of Formula (Ia).

In another embodiment provided herein are compounds of Formula (II):

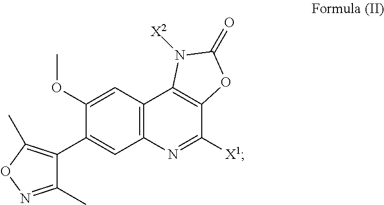

or a pharmaceutically acceptable salt thereof,
wherein:
X¹ is —CH₂NR¹R²;
X² is H, optionally substituted alkyl, optionally substituted alkenyl, optionally substituted cycloalkyl, optionally substituted aryl, optionally substituted heterocycloalkyl, optionally substituted heteroaryl, optionally substituted —CH₂-cycloalkyl, optionally substituted —CH₂-aryl, optionally substituted —CH₂-heterocycloalkyl, optionally substituted —CH₂-heteroaryl, optionally substituted —CH(C₁-C₆-alkyl)-alkyl, optionally substituted —CH(C₁-C₆-alkyl)-cycloalkyl, optionally substituted —CH(C₁-C₆-alkyl)-aryl, optionally substituted —CH(C₁-C₆-alkyl)-heterocycloalkyl, or optionally substituted —CH(C₁-C₆-alkyl)-heteroaryl; and
R¹ and R² are each independently H, C₁-C₁₂alkyl, cycloalkyl, heterocycloalkyl, aryl, or heteroaryl, or —C(O)(C₁-C₆-alkyl).

In another embodiment is a compound of Formula (II) wherein R¹ is H and R² is H. In another embodiment is a compound of Formula (II) wherein R¹ is H and R² is C₁-C₁₂alkyl. In another embodiment is a compound of Formula (II) wherein R¹ is H and R² is C₁-C₆alkyl. In another embodiment is a compound of Formula (II) wherein R¹ is H and R² is methyl. In another embodiment is a compound of Formula (II) wherein R¹ is H and R² is ethyl. In another embodiment is a compound of Formula (II) wherein R¹ is H and R² is propyl. In another embodiment is a compound of Formula (II) wherein R¹ is C₁-C₁₂alkyl and R² is C₁-C₁₂alkyl. In another embodiment is a compound of Formula (II) wherein R¹ is C₁-C₆alkyl and R² is C₁-C₆alkyl. In another embodiment is a compound of Formula (II) wherein R¹ is —CH₃ and R² is —CH₃. In another embodiment is a compound of Formula (II) wherein R¹ is —CH₃ and R² is —CH₂CH₃. In another embodiment is a compound of Formula (II) wherein R¹ is —CH₃ and R² is —CH₂CH₂CH₃. In another embodiment is a compound of Formula (II) wherein R¹ is H and R² is —C(O)(C₁-C₆-alkyl). In another embodiment is a compound of Formula (II) wherein R¹ is H and R² is —C(O)CH₃. In another embodiment is a compound of Formula (II) wherein R¹ is H and R² is —C(O)(C₁-C₆-alkyl). In another embodiment is a compound of Formula (II) wherein R¹ is H and R² is —C(O)CH₂CH₃. In another embodiment is a compound of Formula (II) wherein R¹ is H and R² is —C(O)CH₂CH₂CH₃. In another embodiment is a compound of Formula (II) wherein R¹ is C₁-C₁₂alkyl and R² is —C(O)(C₁-C₆-alkyl). In another embodiment is a compound of Formula (II) wherein R¹ is C₁-C₆alkyl and R² is —C(O)(C₁-C₆-alkyl). In another embodiment is a compound of Formula (II) wherein R¹ is C₁-C₆alkyl and R² is —C(O)CH₃. In another embodiment is a compound of Formula (II) wherein R¹ is C₁-C₆alkyl and R² is —C(O)CH₂CH₃. In another embodiment is a compound of Formula (II) wherein R¹ is C₁-C₆alkyl and R² is —C(O)CH₂CH₂CH₃. In another embodiment is a compound of Formula (II) wherein R¹ is —CH₃ and R² is —C(O)CH₃. In another embodiment is a compound of Formula (II) wherein R¹ is —CH₃ and R² is —C(O)CH₂CH₃. In another embodiment is a compound of Formula (II) wherein R¹ is —CH₃ and R² is —C(O)CH₂CH₂CH₃. In another embodiment is a compound of Formula (II) wherein R¹ is —CH₂CH₃ and R² is —C(O)CH₃. In another embodiment is a compound of Formula (II) wherein R¹ is —CH₂CH₃ and R² is —C(O)CH₂CH₃. In another embodiment is a compound of Formula (II) wherein R¹ is —CH₂CH₃ and R² is —C(O)CH₂CH₂CH₃.

In another embodiment is a compound of Formula (II) wherein X² is H. In another embodiment is a compound of Formula (II) wherein X² is alkenyl. In another embodiment is a compound of Formula (II) wherein X² is allyl. In another embodiment is a compound of Formula (II) wherein X² is —CH₂-aryl optionally substituted with halogen or methoxy. In another embodiment is a compound of Formula (II) wherein X² is —CH₂-aryl optionally substituted with chloro or methoxy. In another embodiment is a compound of Formula (II) wherein X² is unsubstituted —CH₂-aryl. In another embodiment is a compound of Formula (II) wherein X² is benzyl optionally substituted with chloro or methoxy. In another embodiment is a compound of Formula (II) wherein X² is unsubstituted benzyl. In another embodiment is a compound of Formula (II) wherein X² is —CH₂-heteroaryl optionally substituted with halogen, trifluoromethyl, or methoxy. In another embodiment is a compound of Formula (II) wherein X² is —CH₂-pyridinyl or —CH₂-furanyl. In another embodiment is a compound of Formula (II) wherein X² is unsubstituted —CH₂-pyridinyl. In another embodiment is a compound of Formula (II) wherein X² is —CH₂-heterocycloalkyl. In another embodiment is a compound of Formula (II) wherein X² is —CH₂-piperidinyl or —CH₂-tetrahydropyranyl. In some embodiments is a compound of Formula (II) wherein X² is —CH₂-piperidinyl. In some embodiments is a compound of Formula (II) wherein X² is —CH₂-tetrahydropyranyl. In another embodiment is a compound of Formula (II) wherein X² is —CH₂-cycloalkyl. In another embodiment is a compound of Formula (II) wherein X² is —CH₂-cyclohexyl. In another embodiment is a compound of Formula (II) wherein X² is —CH(C₁-C₆-alkyl)-aryl. In another embodiment is a compound of Formula (II) wherein X² is —CH(CH₃)-phenyl. In another embodiment is a compound of Formula (II) wherein X² is aryl. In another embodiment is a compound of Formula (II) wherein X² is phenyl. In another embodiment is a compound of Formula (II) wherein X² is heteroaryl. In another embodiment is a compound of Formula (II) wherein X² is pyridinyl.

In another embodiment is a compound selected from: Ethyl 1-benzyl-7-(3,5-dimethylisoxazol-4-yl)-8-methoxy-2-oxo-oxazolo[5,4-c]quinoline-4-carboxylate, 1-Benzyl-7-(3,5-dimethylisoxazol-4-yl)-8-methoxy-oxazolo[5,4-c]quinolin-2-one, 7-(3,5-Dimethylisoxazol-4-yl)-8-methoxy-1-[(1R)-1-(2-pyridyl)ethyl]oxazolo[5,4-c]quinolin-2-one, 7-(3,5-Dimethylisoxazol-4-yl)-8-methoxy-1-phenyl-oxazolo[5,4-c]quinolin-2-one, 7-(3,5-Dimethylisoxazol-4-yl)-8-methoxy-1-(2-pyridyl)oxazolo[5,4-c]quinolin-2-one, 1-(Cyclohexylmethyl)-7-(3,5-dimethylisoxazol-4-yl)-8-methoxy-oxazolo[5,4-c]quinolin-2-one, 7-(3,5-Dimethylisoxazol-4-yl)-8-methoxy-1-tetrahydropyran-3-yl-oxazolo[5,4-c]quinolin-2-one, 7-(3,5-Dimethylisoxazol-4-yl)-8-methoxy-1-(tetrahydropyran-2-ylmethyl)oxazolo[5,4-c]quinolin-2-one, 7-(3,5-Dimethylisoxazol-4-yl)-8-methoxy-1-(2-piperidylmethyl)oxazolo[5,4-c]quinolin-2-one, 1-allyl-7-(3,5-dimethylisoxazol-4-yl)-8-methoxy-oxazolo[5,4-c]quinolin-2-one, 7-(3,5-dimethylisoxazol-4-yl)-8-methoxy-1H-oxazolo[5,4-c]quinolin-2-one, 7-(3,5-Dimethylisoxazol-4-yl)-8-methoxy-1-(1-phenylethyl)oxazolo[5,4-c]quinolin-2-one, 7-(3,5-Dimethylisoxazol-4-yl)-8-methoxy-1-(3-pyridylmethyl)oxazolo[5,4-c]quinolin-2-one, 7-(3,5-dimethylisoxazol-4-yl)-8-methoxy-1-[[5-(trifluoromethyl)-2-furyl]methyl]oxazolo[5,4-c]quinolin-2-one, 7-(3,5-dimethylisoxazol-4-yl)-8-methoxy-1-(4-pyridylmethyl)oxazolo[5,4-c]quinolin-2-one, 7-(3,5-dimethylisoxazol-4-yl)-8-methoxy-1-(2-pyridylmethyl)oxazolo[5,4-c]quinolin-2-one, 1-[(3-chlorophenyl)methyl]-7-(3,5-dimethylisoxazol-4-yl)-8-methoxy-oxazolo[5,4-c]quinolin-2-one, 1-[(2-chlorophenyl)methyl]-7-(3,5-dimethylisoxazol-4-yl)-8-methoxy-oxazolo[5,4-c]quinolin-2-one, 7-(3,5-dimethylisoxazol-4-yl)-8-methoxy-1-[(3-methoxyphenyl)methyl]oxazolo[5,4-c]quinolin-2-one, 7-(3,5-dimethylisoxazol-4-yl)-8-methoxy-1-[(2-methoxyphenyl)methyl]oxazolo[5,4-c]quinolin-2-one, 1-Benzyl-7-(3,5-dimethylisoxazol-4-yl)-8-methoxy-2-oxo-oxazolo[5,4-c]quinoline-4-carboxylic acid, 1-Benzyl-7-(3,5-dimethylisoxazol-4-yl)-8-methoxy-N-methyl-2-oxo-oxazolo[5,4-c]quinoline-4-carboxamide, 1-benzyl-7-(3,5-dimethylisoxazol-4-yl)-8-methoxy-N,N-dimethyl-2-oxo-oxazolo[5,4-c]quinoline-4-carboxamide, 1-Benzyl-7-(3,5-dimethylisoxazol-4-yl)-8-methoxy-4-(morpholine-4-carbonyl)oxazolo[5,4-c]quinolin-2-one, 1-Benzyl-4-cyclohexyl-7-(3,5-dimethylisoxazol-4-yl)-8-methoxy-oxazolo[5,4-c]quinolin-2-one, 1-Benzyl-7-(3,5-dimethylisoxazol-4-yl)-8-methoxy-4-(4-piperidyl)oxazolo[5,4-c]quinolin-2-one, 1-benzyl-7-(3,5-dimethylisoxazol-4-yl)-8-methoxy-4-(methoxymethyl)oxazolo[5,4-c]quinolin-2-one, 1-Benzyl-7-(3,5-dimethylisoxazol-4-yl)-8-methoxy-4-(phenoxymethyl)oxazolo[5,4-c]quinolin-2-one, 1-Benzyl-7-(3,5-dimethylisoxazol-4-yl)-8-methoxy-4-phenyl-oxazolo[5,4-c]quinolin-2-one, 1-Benzyl-7-(3,5-dimethylisoxazol-4-yl)-4-(1H-imidazol-4-yl)-8-methoxy-oxazolo[5,4-c]quinolin-2-one, 1-Benzyl-7-(3,5-dimethylisoxazol-4-yl)-4-isobutyl-8-methoxy-oxazolo[5,4-c]quinolin-2-one, and 1,4-Dibenzyl-7-(3,5-dimethylisoxazol-4-yl)-8-methoxy-oxazolo[5,4-c]quinolin-2-one; or a pharmaceutically acceptable salt thereof.

In another embodiment is a compound selected from the examples as shown in Table 1.

TABLE 1

| Example | Structure | Nomenclature |
|---|---|---|
| 1 | 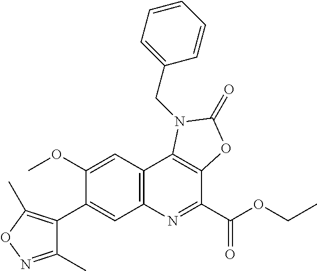 [SMILES: CCOC(=O)c1nc2cc(-c3c(C)noc3C)c(OC)cc2c2c1oc(=O)n2Cc1ccccc1] | Ethyl 1-benzyl-7-(3,5-dimethylisoxazol-4-yl)-8- methoxy-2-oxo-oxazolo[5,4-c]quinoline-4- carboxylate |
| 2 | 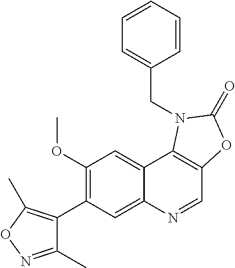 [SMILES: COc1cc2c(cc1-c1c(C)noc1C)ncc1oc(=O)n(Cc3ccccc3)c12] | 1-Benzyl-7-(3,5-dimethylisoxazol-4-yl)-8- methoxy-oxazolo[5,4-c]quinolin-2-one |
| 3 | 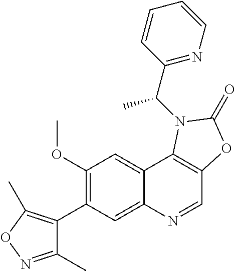 [SMILES: COc1cc2c(cc1-c1c(C)noc1C)ncc1oc(=O)n([C@H](C)c3ccccn3)c12] | 7-(3,5-Dimethylisoxazol-4-yl)-8-methoxy-1- [(1R)-1-(2-pyridyl)ethyl]oxazolo[5,4-c]quinolin- 2-one |
| 4 | 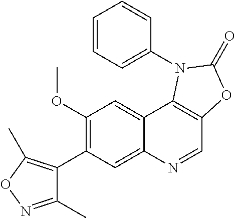 [SMILES: COc1cc2c(cc1-c1c(C)noc1C)ncc1oc(=O)n(-c3ccccc3)c12] | 7-(3,5-Dimethylisoxazol-4-yl)-8-methoxy-1- phenyl-oxazolo[5,4-c]quinolin-2-one |
| 5 | 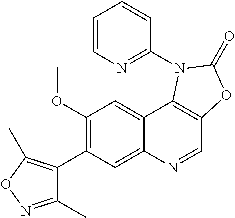 [SMILES: COc1cc2c(cc1-c1c(C)noc1C)ncc1oc(=O)n(-c3ccccn3)c12] | 7-(3,5-Dimethylisoxazol-4-yl)-8-methoxy-1-(2- pyridyl)oxazolo[5,4-c]quinolin-2-one |
| 6 | 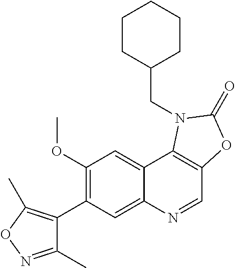 [SMILES: COc1cc2c(cc1-c1c(C)noc1C)ncc1oc(=O)n(CC3CCCCC3)c12] | 1-(Cyclohexylmethyl)-7-(3,5-dimethylisoxazol- 4-yl)-8-methoxy-oxazolo[5,4-c]quinolin-2-one |
| 7 | 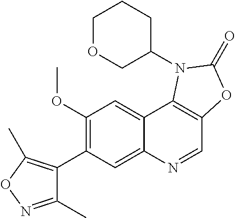 [SMILES: COc1cc2c(cc1-c1c(C)noc1C)ncc1oc(=O)n(C3CCCOC3)c12] | 7-(3,5-Dimethylisoxazol-4-yl)-8-methoxy-1- tetrahydropyran-3-yl-oxazolo[5,4-c]quinolin-2- one |
| 8 | 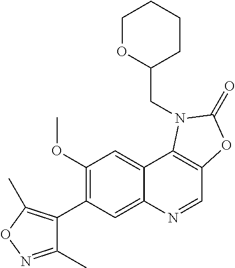 [SMILES: COc1cc2c(cc1-c1c(C)noc1C)ncc1oc(=O)n(CC3CCCCO3)c12] | 7-(3,5-Dimethylisoxazol-4-yl)-8-methoxy-1- (tetrahydropyran-2-ylmethyl)oxazolo[5,4- c]quinolin-2-one |
| 9 | 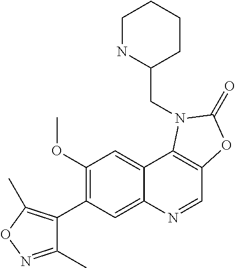 [SMILES: COc1cc2c(cc1-c1c(C)noc1C)ncc1oc(=O)n(CC3CCCCN3)c12] | 7-(3,5-Dimethylisoxazol-4-yl)-8-methoxy-1-(2- piperidylmethyl)oxazolo[5,4-c]quinolin-2-one |
| 10 | 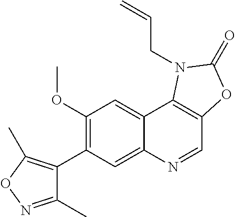 [SMILES: C=CCn1c(=O)oc2cnc3cc(-c4c(C)noc4C)c(OC)cc3c21] | 1-Allyl-7-(3,5-dimethylisoxazol-4-yl)-8- methoxy-oxazolo[5,4-c]quinolin-2-one |
| 11 | 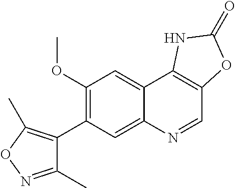 [SMILES: COc1cc2c(cc1-c1c(C)noc1C)ncc1oc(=O)[nH]c12] | 7-(3,5-Dimethylisoxazol-4-yl)-8-methoxy-1H- oxazolo[5,4-c]quinolin-2-one |
| 12 | 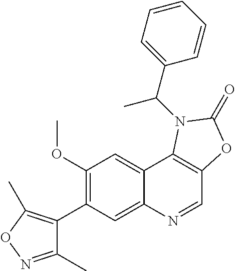 [SMILES: COc1cc2c(cc1-c1c(C)noc1C)ncc1oc(=O)n(C(C)c3ccccc3)c12] | 7-(3,5-Dimethylisoxazol-4-yl)-8-methoxy-1-(1- phenylethyl)oxazolo[5,4-c]quinolin-2-one |
| 12A | 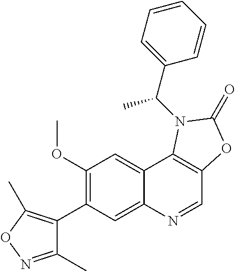 [SMILES: COc1cc2c(cc1-c1c(C)noc1C)ncc1oc(=O)n([C@H](C)c3ccccc3)c12] | 7-(3,5-Dimethylisoxazol-4-yl)-8-methoxy-1- [(1R)-1-phenylethyl]oxazolo[5,4-c]quinolin-2- one |
| 13 | 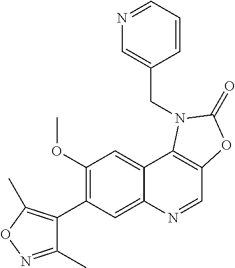 [SMILES: COc1cc2c(cc1-c1c(C)noc1C)ncc1oc(=O)n(Cc3cccnc3)c12] | 7-(3,5-Dimethylisoxazol-4-yl)-8-methoxy-1-(3- pyridylmethyl)oxazolo[5,4-c]quinolin-2-one |
| 14 | 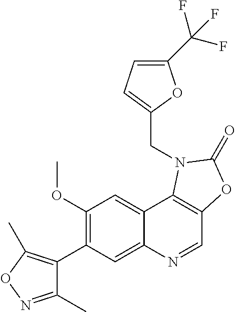 [SMILES: COc1cc2c(cc1-c1c(C)noc1C)ncc1oc(=O)n(Cc3ccc(C(F)(F)F)o3)c12] | 7-(3,5-Dimethylisoxazol-4-yl)-8-methoxy-1-[[5- (trifluoromethyl)-2-furyl]methyl]oxazolo[5,4- c]quinolin-2-one |
| 15 | 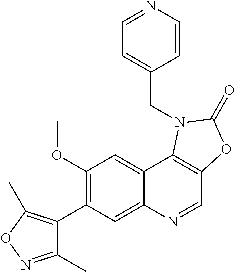 [SMILES: COc1cc2c(cc1-c1c(C)noc1C)ncc1oc(=O)n(Cc3ccncc3)c12] | 7-(3,5-Dimethylisoxazol-4-yl)-8-methoxy-1-(4- pyridylmethyl)oxazolo[5,4-c]quinolin-2-one |
| 16 | 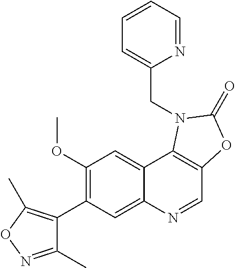 [SMILES: COc1cc2c(cc1-c1c(C)noc1C)ncc1oc(=O)n(Cc3ccccn3)c12] | 7-(3,5-Dimethylisoxazol-4-yl)-8-methoxy-1-(2- pyridylmethyl)oxazolo[5,4-c]quinolin-2-one |
| 17 | 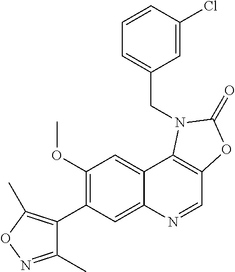 [SMILES: COc1cc2c(cc1-c1c(C)noc1C)ncc1oc(=O)n(Cc3cccc(Cl)c3)c12] | 1-[(3-Chlorophenyl)methyl]-7-(3,5- dimethylisoxazol-4-yl)-8-methoxy-oxazolo[5,4- c]quinolin-2-one |
| 18 | 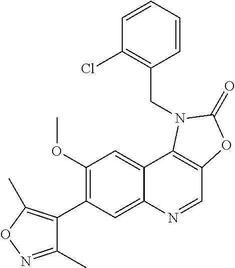 [SMILES: COc1cc2c(cc1-c1c(C)noc1C)ncc1oc(=O)n(Cc3ccccc3Cl)c12] | 1-[(2-Chlorophenyl)methyl]-7-(3,5- dimethylisoxazol-4-yl)-8-methoxy-oxazolo[5,4- c]quinolin-2-one |
| 19 | 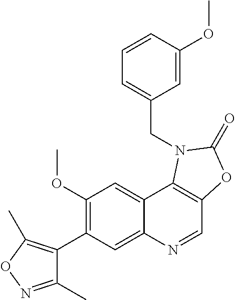 [SMILES: COc1cccc(Cn2c(=O)oc3cnc4cc(-c5c(C)noc5C)c(OC)cc4c32)c1] | 7-(3,5-Dimethylisoxazol-4-yl)-8-methoxy-1-[(3- methoxyphenyl)methyl]oxazolo[5,4-c]quinolin- 2-one |
| 20 | 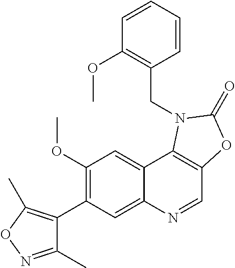 [SMILES: COc1ccccc1Cn1c(=O)oc2cnc3cc(-c4c(C)noc4C)c(OC)cc3c21] | 7-(3,5-dimethylisoxazol-4-yl)-8-methoxy-1-[(2- methoxyphenyl)methyl]oxazolo[5,4-c]quinolin- 2-one |
| 21 | 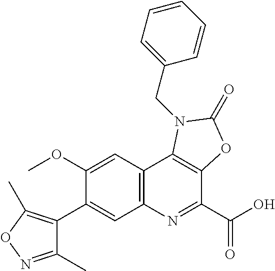 [SMILES: COc1cc2c(cc1-c1c(C)noc1C)nc(C(=O)O)c1oc(=O)n(Cc3ccccc3)c12] | 1-Benzyl-7-(3,5-dimethylisoxazol-4-yl)-8- methoxy-2-oxo-oxazolo[5,4-c]quinoline-4- carboxylic acid |
| 22 | 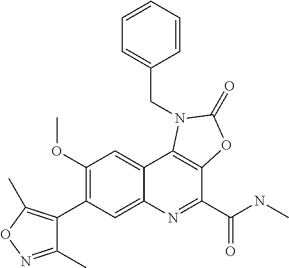 [SMILES: CNC(=O)c1nc2cc(-c3c(C)noc3C)c(OC)cc2c2c1oc(=O)n2Cc1ccccc1] | 1-Benzyl-7-(3,5-dimethylisoxazol-4-yl)-8- methoxy-N-methyl-2-oxo-oxazolo[5,4- c]quinoline-4-carboxamide |
| 23 | 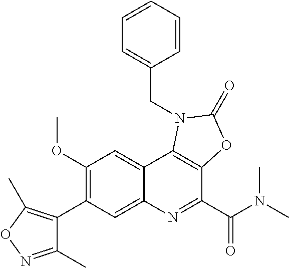 [SMILES: COc1cc2c(cc1-c1c(C)noc1C)nc(C(=O)N(C)C)c1oc(=O)n(Cc3ccccc3)c12] | 1-Benzyl-7-(3,5-dimethylisoxazol-4-yl)-8- methoxy-N,N-dimethyl-2-oxo-oxazolo[5,4- c]quinoline-4-carboxamide |
| 24 | 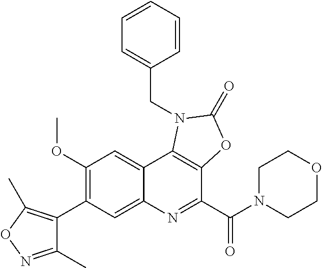 [SMILES: COc1cc2c(cc1-c1c(C)noc1C)nc(C(=O)N1CCOCC1)c1oc(=O)n(Cc3ccccc3)c12] | 1-Benzyl-7-(3,5-dimethylisoxazol-4-yl)-8- methoxy-4-(morpholine-4-carbonyl)oxazolo[5,4- c]quinolin-2-one |
| 25 | 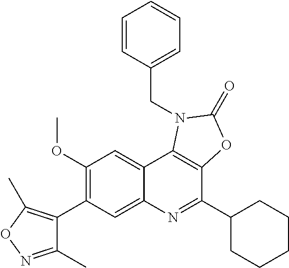 [SMILES: COc1cc2c(cc1-c1c(C)noc1C)nc(C1CCCCC1)c1oc(=O)n(Cc3ccccc3)c12] | 1-Benzyl-4-cyclohexyl-7-(3,5-dimethylisoxazol- 4-yl)-8-methoxy-oxazolo[5,4-c]quinolin-2-one |
| 26 | 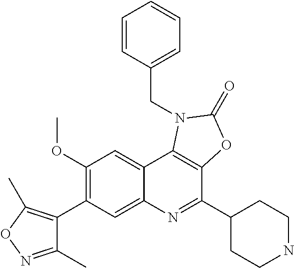 [SMILES: COc1cc2c(cc1-c1c(C)noc1C)nc(C1CCNCC1)c1oc(=O)n(Cc3ccccc3)c12] | 1-Benzyl-7-(3,5-dimethylisoxazol-4-yl)-8- methoxy-4-(4-piperidyl)oxazolo[5,4-c]quinolin- 2-one |
| 27 | 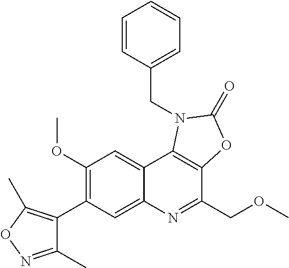 [SMILES: COCc1nc2cc(-c3c(C)noc3C)c(OC)cc2c2c1oc(=O)n2Cc1ccccc1] | 1-Benzyl-7-(3,5-dimethylisoxazol-4-yl)-8- methoxy-4-(methoxymethyl)oxazolo[5,4- c]quinolin-2-one |
| 28 | 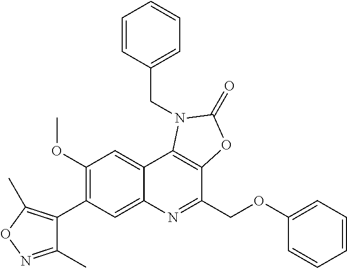 [SMILES: COc1cc2c(cc1-c1c(C)noc1C)nc(COc1ccccc1)c1oc(=O)n(Cc3ccccc3)c12] | 1-Benzyl-7-(3,5-dimethylisoxazol-4-yl)-8- methoxy-4-(phenoxymethyl)oxazolo[5,4- c]quinolin-2-one |
| 29 | 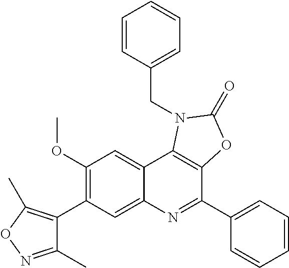 [SMILES: COc1cc2c(cc1-c1c(C)noc1C)nc(-c1ccccc1)c1oc(=O)n(Cc3ccccc3)c12] | 1-Benzyl-7-(3,5-dimethylisoxazol-4-yl)-8- methoxy-4-phenyl-oxazolo[5,4-c]quinolin-2-one |
| 30 | 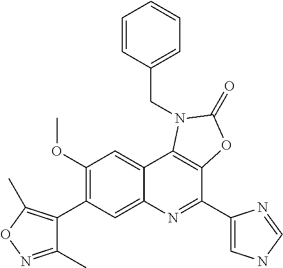 [SMILES: COc1cc2c(cc1-c1c(C)noc1C)nc(-c1c[nH]cn1)c1oc(=O)n(Cc3ccccc3)c12] | 1-Benzyl-7-(3,5-dimethylisoxazol-4-yl)-4-(1H- imidazol-4-yl)-8-methoxy-oxazolo[5,4- c]quinolin-2-one |
| 31 | 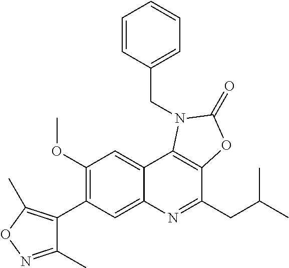 [SMILES: COc1cc2c(cc1-c1c(C)noc1C)nc(CC(C)C)c1oc(=O)n(Cc3ccccc3)c12] | 1-Benzyl-7-(3,5-dimethylisoxazol-4-yl)-4- isobutyl-8-methoxy-oxazolo[5,4-c]quinolin-2- one |
| 32 | 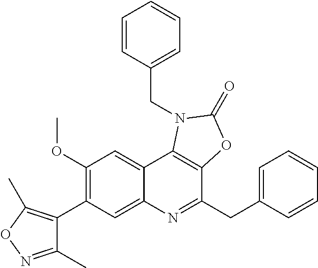 [SMILES: COc1cc2c(cc1-c1c(C)noc1C)nc(Cc1ccccc1)c1oc(=O)n(Cc3ccccc3)c12] | 1,4-Dibenzyl-7-(3,5-dimethylisoxazol-4-yl)-8- methoxy-oxazolo[5,4-c]quinolin-2-one |
| 33 | 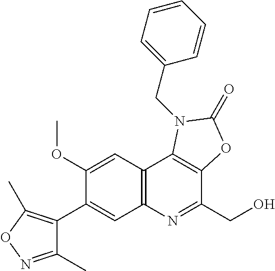 [SMILES: COc1cc2c(cc1-c1c(C)noc1C)nc(CO)c1oc(=O)n(Cc3ccccc3)c12] | 1-Benzyl-7-(3,5-dimethylisoxazol-4-yl)-4- (hydroxymethyl)-8-methoxy-oxazolo[5,4- c]quinolin-2-one |
| 34 | 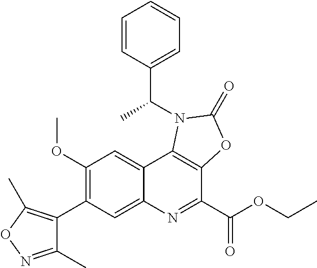 [SMILES: CCOC(=O)c1nc2cc(-c3c(C)noc3C)c(OC)cc2c2c1oc(=O)n2[C@H](C)c1ccccc1] | Ethyl 7-(3,5-dimethylisoxazol-4-yl)-8-methoxy- 2-oxo-1-[(1R)-1-phenylethyl]oxazolo[5,4- c]quinoline-4-carboxylate |
| 35 | 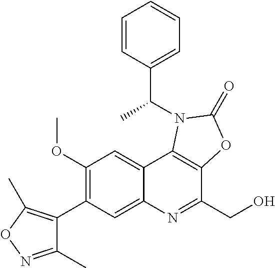 [SMILES: COc1cc2c(cc1-c1c(C)noc1C)nc(CO)c1oc(=O)n([C@H](C)c3ccccc3)c12] | 7-(3,5-Dimethylisoxazol-4-yl)-4- (hydroxymethyl)-8-methoxy-1-[(1R)-1- phenylethyl]oxazolo[5,4-c]quinolin-2-one |
| 36 | 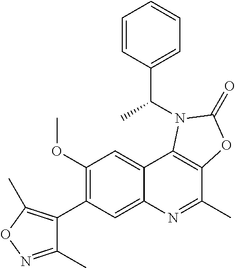 [SMILES: COc1cc2c(cc1-c1c(C)noc1C)nc(C)c1oc(=O)n([C@H](C)c3ccccc3)c12] | 7-(3,5-Dimethylisoxazol-4-yl)-8-methoxy-4- methyl-1-[(1R)-1-phenylethyl]oxazolo[5,4- c]quinolin-2-one |
| 37 | 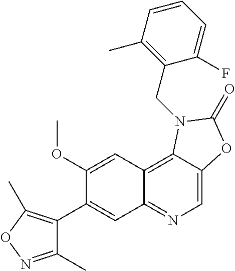 [SMILES: COc1cc2c(cc1-c1c(C)noc1C)ncc1oc(=O)n(Cc3c(C)cccc3F)c12] | 7-(3,5-Dimethylisoxazol-4-yl)-1-[(2-fluoro-6- methyl-phenyl)methyl]-8-methoxy-oxazolo[5,4- c]quinolin-2-one |
| 38 | 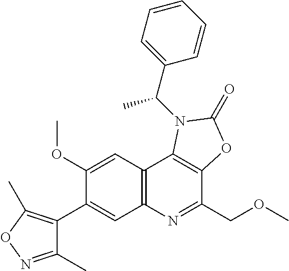 [SMILES: COCc1nc2cc(-c3c(C)noc3C)c(OC)cc2c2c1oc(=O)n2[C@H](C)c1ccccc1] | 7-(3,5-Dimethylisoxazol-4-yl)-8-methoxy-4- (methoxymethyl)-1-[(1R)-1- phenylethyl]oxazolo[5,4-c]quinolin-2-one |
| 39 | 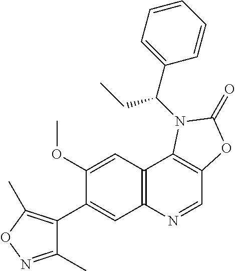 [SMILES: CC[C@H](c1ccccc1)n1c(=O)oc2cnc3cc(-c4c(C)noc4C)c(OC)cc3c21] | 7-(3,5-Dimethylisoxazol-4-yl)-8-methoxy-1- [(1R)-1-phenylpropyl]oxazolo[5,4-c]quinolin-2- one |
| 40 | 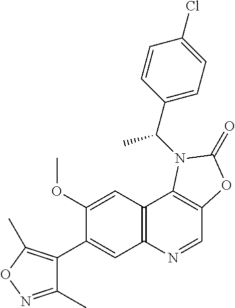 [SMILES: COc1cc2c(cc1-c1c(C)noc1C)ncc1oc(=O)n([C@H](C)c3ccc(Cl)cc3)c12] | Ethyl 1-[(1R)-1-(4-chlorophenyl)ethyl]-7-(3,5- dimethylisoxazol-4-yl)-8-methoxy-oxazolo[5,4- c]quinolin-2-one |
| 41 | 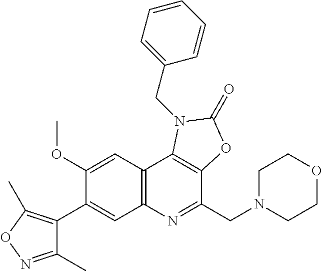 [SMILES: COc1cc2c(cc1-c1c(C)noc1C)nc(CN1CCOCC1)c1oc(=O)n(Cc3ccccc3)c12] | 1-Benzyl-7-(3,5-dimethylisoxazol-4-yl)-8- methoxy-4-(morpholinomethyl)oxazolo[5,4- c]quinolin-2-one |
| 42 | 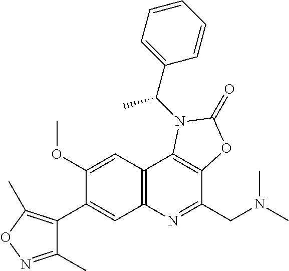 [SMILES: COc1cc2c(cc1-c1c(C)noc1C)nc(CN(C)C)c1oc(=O)n([C@H](C)c3ccccc3)c12] | 4-(Dimethylaminomethyl)-7-(3,5- dimethylisoxazol-4-yl)-8-methoxy-1-[(1R)-1- phenylethyl]oxazolo[5,4-c]quinolin-2-one |
| 43 | 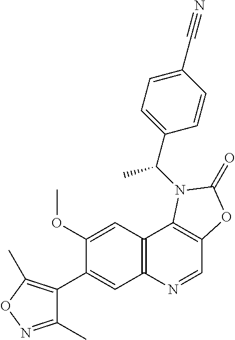 [SMILES: COc1cc2c(cc1-c1c(C)noc1C)ncc1oc(=O)n([C@H](C)c3ccc(C#N)cc3)c12] | 4-[(1R)-1-[7-(3,5-Dimethylisoxazol-4-yl)-8- methoxy-2-oxo-oxazolo[5,4-c]quinolin-1- yl]ethyl]benzonitrile |
| 44 | 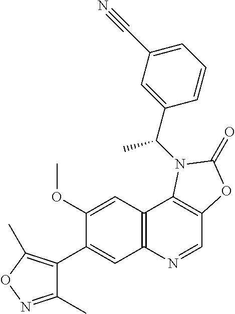 [SMILES: COc1cc2c(cc1-c1c(C)noc1C)ncc1oc(=O)n([C@H](C)c3cccc(C#N)c3)c12] | 3-[(1R)-1-[7-(3,5-Dimethylisoxazol-4-yl)-8- methoxy-2-oxo-oxazolo[5,4-c]quinolin-1- yl]ethyl]benzonitrile |
| 45 | 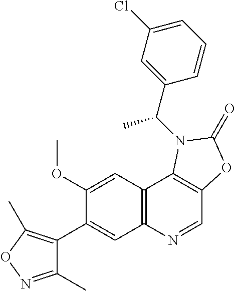 [SMILES: COc1cc2c(cc1-c1c(C)noc1C)ncc1oc(=O)n([C@H](C)c3cccc(Cl)c3)c12] | 1-[(1R)-1-(3-Chlorophenyl)ethyl]-7-(3,5- dimethylisoxazol-4-yl)-8-methoxy-oxazolo[5,4- c]quinolin-2-one |
| 46 | 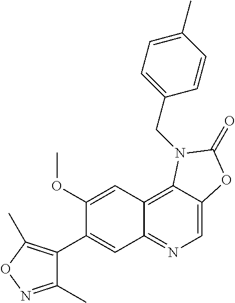 [SMILES: COc1cc2c(cc1-c1c(C)noc1C)ncc1oc(=O)n(Cc3ccc(C)cc3)c12] | 7-(3,5-Dimethylisoxazol-4-yl)-8-methoxy-1-(p- tolylmethyl)oxazolo[5,4-c]quinolin-2-one |
| 47 | 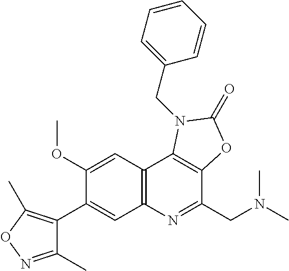 [SMILES: COc1cc2c(cc1-c1c(C)noc1C)nc(CN(C)C)c1oc(=O)n(Cc3ccccc3)c12] | 1-Benzyl-4-(dimethylaminomethyl)-7-(3,5- dimethylisoxazol-4-yl)-8-methoxy-oxazolo[5,4- c]quinolin-2-one |
| 48 | 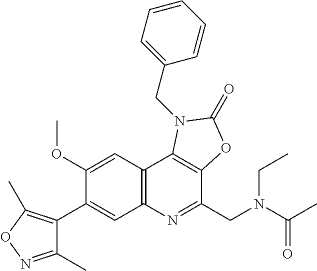 [SMILES: CCN(Cc1nc2cc(-c3c(C)noc3C)c(OC)cc2c2c1oc(=O)n2Cc1ccccc1)C(C)=O] | N-[[1-Benzyl-7-(3,5-dimethylisoxazol-4-yl)-8- methoxy-2-oxo-oxazolo[5,4-c]quinolin-4- yl]methyl]-N-ethyl-acetamide |
| 49 | 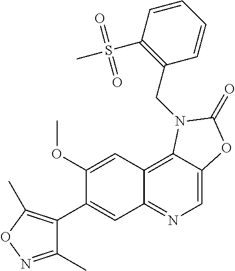 [SMILES: COc1cc2c(cc1-c1c(C)noc1C)ncc1oc(=O)n(Cc3ccccc3S(C)(=O)=O)c12] | 7-(3,5-Dimethylisoxazol-4-yl)-8-methoxy-1-[(2- methylsulfonylphenyl)methyl]oxazolo[5,4- c]quinolin-2-one |
| 50 | 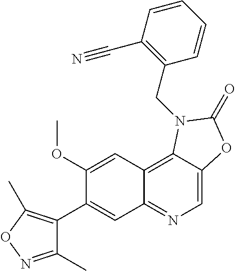 [SMILES: COc1cc2c(cc1-c1c(C)noc1C)ncc1oc(=O)n(Cc3ccccc3C#N)c12] | 2-[[7-(3,5-Dimethylisoxazol-4-yl)-8-methoxy-2- oxo-oxazolo[5,4-c]quinolin-1- yl]methyl]benzonitrile |
| 51 | 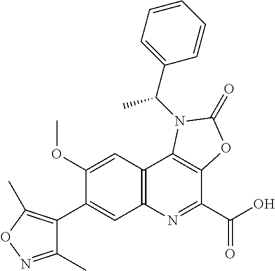 [SMILES: COc1cc2c(cc1-c1c(C)noc1C)nc(C(=O)O)c1oc(=O)n([C@H](C)c3ccccc3)c12] | 7-(3,5-Dimethylisoxazol-4-yl)-8-methoxy-2- oxo-1-[(1R)-1-phenylethyl]oxazolo[5,4- c]quinoline-4-carboxylic acid |
| 52 | 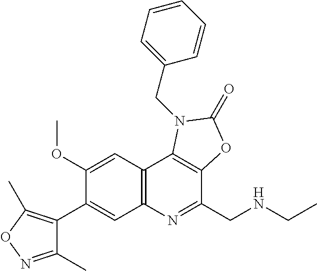 [SMILES: CCNCc1nc2cc(-c3c(C)noc3C)c(OC)cc2c2c1oc(=O)n2Cc1ccccc1] | 1-Benzyl-7-(3,5-dimethylisoxazol-4-yl)-4- (ethylaminomethyl)-8-methoxy-oxazolo[5,4- c]quinolin-2-one |
| 53 | 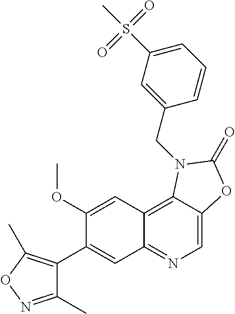 [SMILES: COc1cc2c(cc1-c1c(C)noc1C)ncc1oc(=O)n(Cc3cccc(S(C)(=O)=O)c3)c12] | 7-(3,5-Dimethylisoxazol-4-yl)-8-methoxy-1-[(3- methylsulfonylphenyl)methyl]oxazolo[5,4- c]quinolin-2-one |
| 54 | 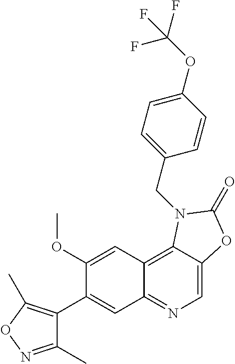 [SMILES: COc1cc2c(cc1-c1c(C)noc1C)ncc1oc(=O)n(Cc3ccc(OC(F)(F)F)cc3)c12] | 7-(3,5-Dimethylisoxazol-4-yl)-8-methoxy-1-[[4- (trifluoromethoxy)phenyl]methyl]oxazolo[5,4- c]quinolin-2-one |
| 55 | 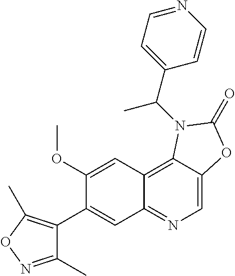 [SMILES: COc1cc2c(cc1-c1c(C)noc1C)ncc1oc(=O)n(C(C)c3ccncc3)c12] | 7-(3,5-Dimethylisoxazol-4-yl)-8-methoxy-1-[1- (4-pyridyl)ethyl]oxazolo[5,4-c]quinolin-2-one |
| 56 | 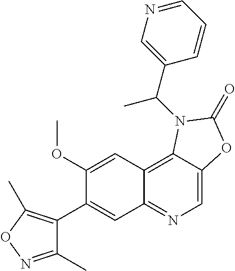 [SMILES: COc1cc2c(cc1-c1c(C)noc1C)ncc1oc(=O)n(C(C)c3cccnc3)c12] | 7-(3,5-dimethylisoxazol-4-yl)-8-methoxy-1-[1- (3-pyridyl)ethyl]oxazolo[5,4-c]quinolin-2-one |
| 57 | 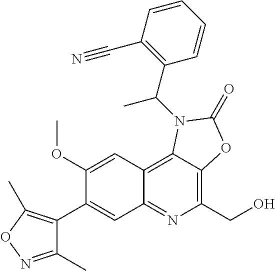 [SMILES: COc1cc2c(cc1-c1c(C)noc1C)nc(CO)c1oc(=O)n(C(C)c3ccccc3C#N)c12] | 2-[[7-(3,5-Dimethylisoxazol-4-yl)-4- (hydroxymethyl)-8-methoxy-2-oxo-oxazolo[5,4- c]quinolin-l-yl]methyl]benzonitrile |
| 58 | 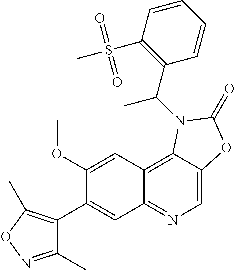 [SMILES: COc1cc2c(cc1-c1c(C)noc1C)ncc1oc(=O)n(C(C)c3ccccc3S(C)(=O)=O)c12] | 7-(3,5-Dimethylisoxazol-4-yl)-8-methoxy-1- [(1R)-1-(2- methylsulfonylphenyl)ethyl]oxazolo[5,4- c]quinolin-2-one |
| 59 | 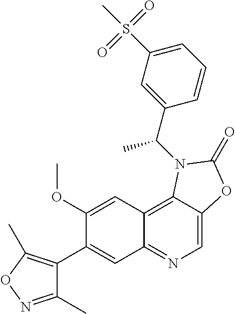 [SMILES: COc1cc2c(cc1-c1c(C)noc1C)ncc1oc(=O)n([C@H](C)c3cccc(S(C)(=O)=O)c3)c12] | 7-(3,5-Dimethylisoxazol-4-yl)-8-methoxy-l- [(1R)-1-(3- methylsulfonylphenyl)ethyl]oxazolo[5,4- c]quinolin-2-one |
| 60 | 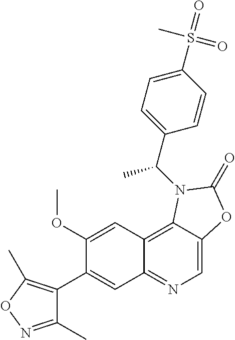 [SMILES: COc1cc2c(cc1-c1c(C)noc1C)ncc1oc(=O)n([C@H](C)c3ccc(S(C)(=O)=O)cc3)c12] | 7-(3,5-Dimethylisoxazol-4-yl)-8-methoxy-1- [(1R)-1-(4- methylsulfonylphenyl)ethyl]oxazolo[5,4- c]quinolin-2-one |
| 61 | 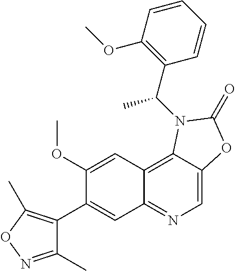 [SMILES: COc1cc2c(cc1-c1c(C)noc1C)ncc1oc(=O)n([C@H](C)c3ccccc3OC)c12] | 7-(3,5-Dimethylisoxazol-4-yl)-8-methoxy-1- [(1R)-1-(2-methoxyphenyl)ethyl]oxazolo[5,4- c]quinolin-2-one |
| 62 | 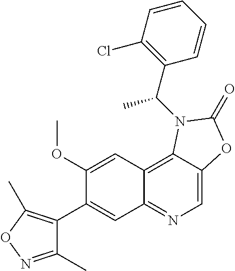 [SMILES: COc1cc2c(cc1-c1c(C)noc1C)ncc1oc(=O)n([C@H](C)c3ccccc3Cl)c12] | 1-[(1R)-1-(2-Chlorophenyl)ethyl]-7-(3,5- dimethylisoxazol-4-yl)-8-methoxy-oxazolo[5,4- c]quinolin-2-one |
| 63 | 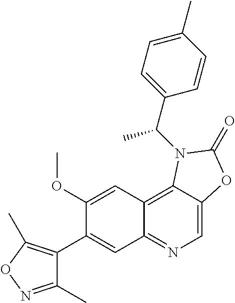 [SMILES: COc1cc2c(cc1-c1c(C)noc1C)ncc1oc(=O)n([C@H](C)c3ccc(C)cc3)c12] | 7-(3,5-Dimethylisoxazol-4-yl)-8-methoxy-1- [(1R)-1-(p-tolyl)ethyl]oxazolo[5,4-c]quinolin-2- one |
| 64 | 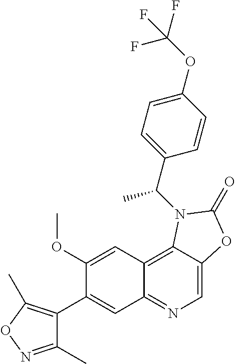 [SMILES: COc1cc2c(cc1-c1c(C)noc1C)ncc1oc(=O)n([C@H](C)c3ccc(OC(F)(F)F)cc3)c12] | 7-(3,5-Dimethylisoxazol-4-yl)-8-methoxy-1- [(1R)-1-]4- (trifluoromethoxy)phenyl]ethyl]oxazolo[5,4- c]quinolin-2-one |
| 65 | 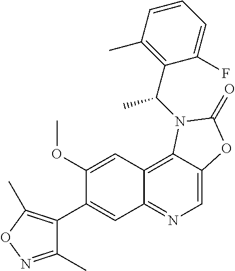 [SMILES: COc1cc2c(cc1-c1c(C)noc1C)ncc1oc(=O)n([C@H](C)c3c(C)cccc3F)c12] | 7-(3,5-Dimethylisoxazol-4-yl)-1-[(1R)-1-(2- fluoro-6-methyl-phenyl)ethyl]-8-methoxy- oxazolo[5,4-c]quinolin-2-one |
| 66 | 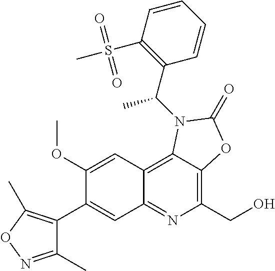 [SMILES: COc1cc2c(cc1-c1c(C)noc1C)nc(CO)c1oc(=O)n([C@H](C)c3ccccc3S(C)(=O)=O)c12] | 7-(3,5-Dimethylisoxazol-4-yl)-4- (hydroxymethyl)-8-methoxy-1-[(1R)-1-(2- methylsulfonylphenyl)ethyl]oxazolo[5,4- c]quinolin-2-one |
| 67 | 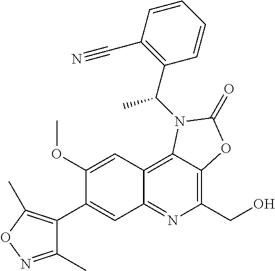 [SMILES: COc1cc2c(cc1-c1c(C)noc1C)nc(CO)c1oc(=O)n([C@H](C)c3ccccc3C#N)c12] | 2-[(1R)-1-[7-(3,5-Dimethylisoxazol-4-yl)-4- (hydroxymethyl)-8-methoxy-2-oxo-oxazolo[5,4- c]quinolin-l-yl]ethyl]benzonitrile |
| 68 | 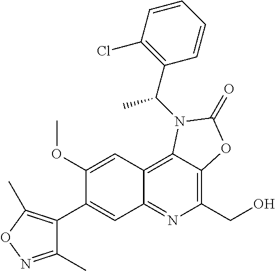 [SMILES: COc1cc2c(cc1-c1c(C)noc1C)nc(CO)c1oc(=O)n([C@H](C)c3ccccc3Cl)c12] | 1-[(1R)-1-(2-Chlorophenyl)ethyl]-7-(3,5- dimethylisoxazol-4-yl)-4-(hydroxymethyl)-8- methoxy-oxazolo[5,4-c]quinolin-2-one |
| 69 | 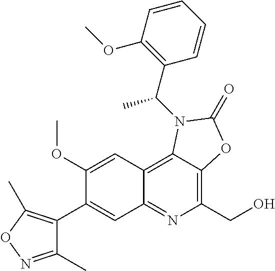 [SMILES: COc1cc2c(cc1-c1c(C)noc1C)nc(CO)c1oc(=O)n([C@H](C)c3ccccc3OC)c12] | 7-(3,5-Dimethylisoxazol-4-yl)-4- (hydroxymethyl)-8-methoxy-1-[(1R)-1-(2- methoxyphenyl)ethyl]oxazolo[5,4-c]quinolin-2- one |
| 70 | 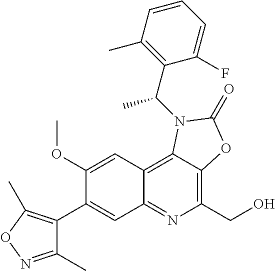 [SMILES: COc1cc2c(cc1-c1c(C)noc1C)nc(CO)c1oc(=O)n([C@H](C)c3c(C)cccc3F)c12] | 7-(3,5-Dimethylisoxazol-4-yl)-1-[(1R)-1-(2- fluoro-6-methyl-phenyl)ethyl]-4- (hydroxymethyl)-8-methoxy-oxazolo[5,4- c]quinolin-2-one |
| 71 | 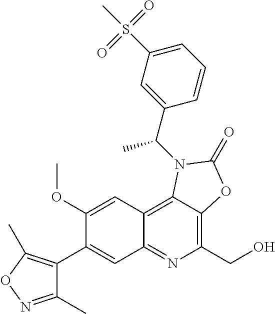 [SMILES: COc1cc2c(cc1-c1c(C)noc1C)nc(CO)c1oc(=O)n([C@H](C)c3cccc(S(C)(=O)=O)c3)c12] | 7-(3,5-Dimethylisoxazol-4-yl)-4- (hydroxymethyl)-8-methoxy-1-[(1R)-1-(3- methylsulfonylphenyl)ethyl]oxazolo[5,4- c]quinolin-2-one |
| 72 | 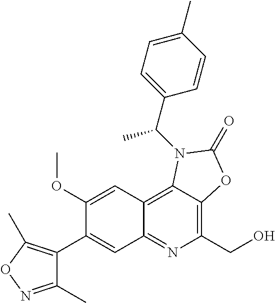 [SMILES: COc1cc2c(cc1-c1c(C)noc1C)nc(CO)c1oc(=O)n([C@H](C)c3ccc(C)cc3)c12] | 7-(3,5-Dimethylisoxazol-4-yl)-4- (hydroxymethyl)-8-methoxy-1-[(1R)-1-(p- tolyl)ethyl]oxazolo[5,4-c]quinolin-2-one |
| 73 | 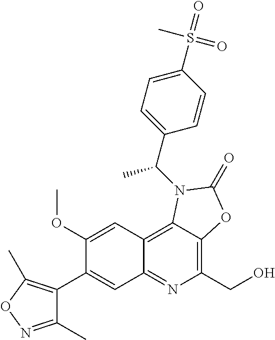 [SMILES: COc1cc2c(cc1-c1c(C)noc1C)nc(CO)c1oc(=O)n([C@H](C)c3ccc(S(C)(=O)=O)cc3)c12] | 7-(3,5-Dimethylisoxazol-4-yl)-4- (hydroxymethyl)-8-methoxy-1-[(1R)-1-(4- methylsulfonylphenyl)ethyl]oxazolo[5,4- c]quinolin-2-one |
| 74 | 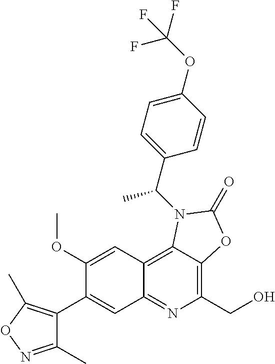 [SMILES: COc1cc2c(cc1-c1c(C)noc1C)nc(CO)c1oc(=O)n([C@H](C)c3ccc(OC(F)(F)F)cc3)c12] | 7-(3,5-Dimethylisoxazol-4-yl)-4- (hydroxymethyl)-8-methoxy-1-[(1R)-1-[4- (trifluoromethoxy)phenyl]ethyl]oxazolo[5,4- c]quinolin-2-one |
| 75 | 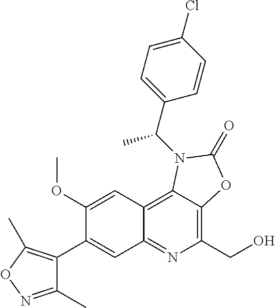 [SMILES: COc1cc2c(cc1-c1c(C)noc1C)nc(CO)c1oc(=O)n([C@H](C)c3ccc(Cl)cc3)c12] | 1-[(1R)-1-(4-Chlorophenyl)ethyl]-7-(3,5- dimethylisoxazol-4-yl)-4-(hydroxymethyl)-8- methoxy-oxazolo[5,4-c]quinolin-2-one |
| 76 | 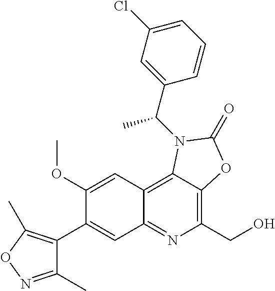 [SMILES: COc1cc2c(cc1-c1c(C)noc1C)nc(CO)c1oc(=O)n([C@H](C)c3cccc(Cl)c3)c12] | 1-[(1R)-1-(3-Chlorophenyl)ethyl]-7-(3,5- dimethylisoxazol-4-yl)-4-(hydroxymethyl)-8- methoxy-oxazolo[5,4-c]quinolin-2-one |
| 77 | 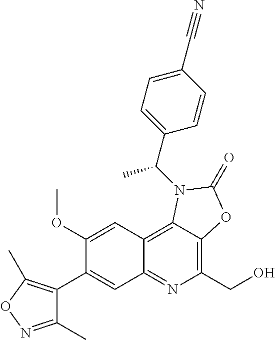 [SMILES: COc1cc2c(cc1-c1c(C)noc1C)nc(CO)c1oc(=O)n([C@H](C)c3ccc(C#N)cc3)c12] | 4-[(1R)-1-[7-(3,5-Dimethylisoxazol-4-yl)-4- (hydroxymethyl)-8-methoxy-2-oxo-oxazolo[5,4- c]quinolin-l-yl]ethyl]benzonitrile |
| 78 | 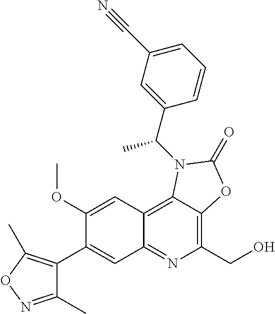 [SMILES: COc1cc2c(cc1-c1c(C)noc1C)nc(CO)c1oc(=O)n([C@H](C)c3cccc(C#N)c3)c12] | 3-[(1R)-1-[7-(3,5-Dimethylisoxazol-4-yl)-4- (hydroxymethyl)-8-methoxy-2-oxo-oxazolo[5,4- c]quinolin-1-yl]ethyl]benzonitrile |
| 79 | 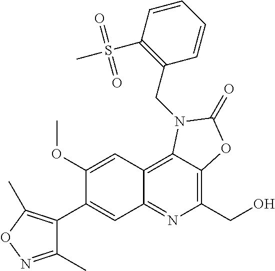 [SMILES: COc1cc2c(cc1-c1c(C)noc1C)nc(CO)c1oc(=O)n(Cc3ccccc3S(C)(=O)=O)c12] | 7-(3,5-Dimethylisoxazol-4-yl)-4- (hydroxymethyl)-8-methoxy-1-[(2- methylsulfonylphenyl)methyl]oxazolo[5,4- c]quinolin-2-one |
| 80 | 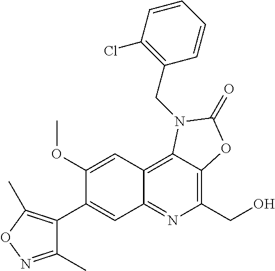 [SMILES: COc1cc2c(cc1-c1c(C)noc1C)nc(CO)c1oc(=O)n(Cc3ccccc3Cl)c12] | 1-[(2-Chlorophenyl)methyl]-7-(3,5- dimethylisoxazol-4-yl)-4-(hydroxymethyl)-8- methoxy-oxazolo[5,4-c]quinolin-2-one |
| 81 | 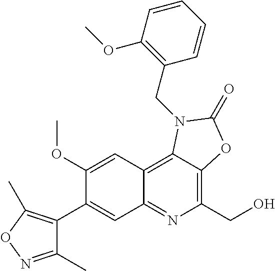 [SMILES: COc1ccccc1Cn1c(=O)oc2c(CO)nc3cc(-c4c(C)noc4C)c(OC)cc3c21] | 7-(3,5-Dimethylisoxazol-4-yl)-4- (hydroxymethyl)-8-methoxy-1-[(2- methoxyphenyl)methyl]oxazolo[5,4-c]quinolin- 2-one |
| 82 | 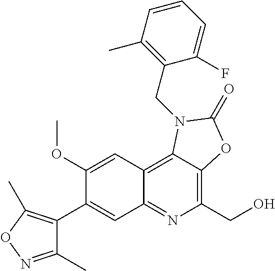 [SMILES: COc1cc2c(cc1-c1c(C)noc1C)nc(CO)c1oc(=O)n(Cc3c(C)cccc3F)c12] | (3,5-Dimethylisoxazol-4-yl)-1-[(2-fluoro-6- methyl-phenyl)methyl]-4-(hydroxymethyl)-8- methoxy-oxazolo[5,4-c]quinolin-2-one |
| 83 | 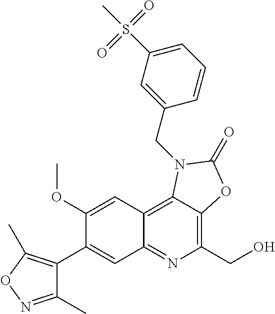 [SMILES: COc1cc2c(cc1-c1c(C)noc1C)nc(CO)c1oc(=O)n(Cc3cccc(S(C)(=O)=O)c3)c12] | 7-(3,5-Dimethylisoxazol-4-yl)-4- (hydroxymethyl)-8-methoxy-1-[(3- methylsulfonylphenyl)methyl]oxazolo[5,4- c]quinolin-2-one |
| 84 | 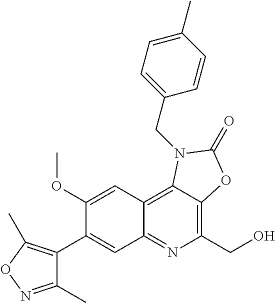 [SMILES: COc1cc2c(cc1-c1c(C)noc1C)nc(CO)c1oc(=O)n(Cc3ccc(C)cc3)c12] | 7-(3,5-Dimethylisoxazol-4-yl)-4- (hydroxymethyl)-8-methoxy-1-(p- tolylmethyl)oxazolo[5,4-c]quinolin-2-one |
| 85 | 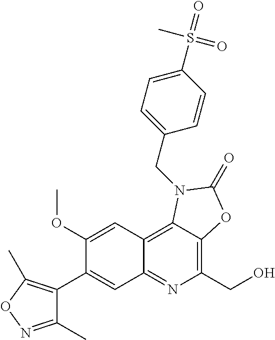 [SMILES: COc1cc2c(cc1-c1c(C)noc1C)nc(CO)c1oc(=O)n(Cc3ccc(S(C)(=O)=O)cc3)c12] | 7-(3,5-Dimethylisoxazol-4-yl)-4- (hydroxymethyl)-8-methoxy-1-[(4- methylsulfonylphenyl)methyl]oxazolo[5,4- c]quinolin-2-one |
| 86 | 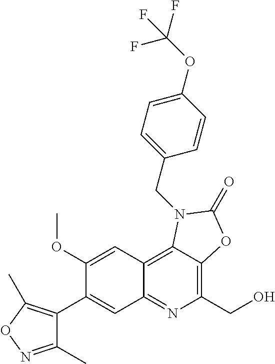 [SMILES: COc1cc2c(cc1-c1c(C)noc1C)nc(CO)c1oc(=O)n(Cc3ccc(OC(F)(F)F)cc3)c12] | 7-(3,5-Dimethylisoxazol-4-yl)-4- (hydroxymethyl)-8-methoxy-1-[[4- (trifluoromethoxy)phenyl]methyl]oxazolo[5,4- c]quinolin-2-one |
| 87 | 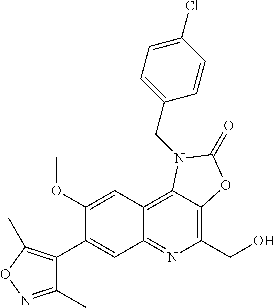 [SMILES: COc1cc2c(cc1-c1c(C)noc1C)nc(CO)c1oc(=O)n(Cc3ccc(Cl)cc3)c12] | 1-[(4-Chlorophenyl)methyl]-7-(3,5- dimethylisoxazol-4-yl)-4-(hydroxymethyl)-8- methoxy-oxazolo[5,4-c]quinolin-2-one |
| 88 | 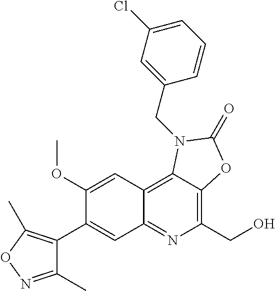 [SMILES: COc1cc2c(cc1-c1c(C)noc1C)nc(CO)c1oc(=O)n(Cc3cccc(Cl)c3)c12] | 1-[(3-Chlorophenyl)methyl]-7-(3,5- dimethylisoxazol-4-yl)-4-(hydroxymethyl)-8- methoxy-oxazolo[5,4-c]quinolin-2-one |
| 89 | 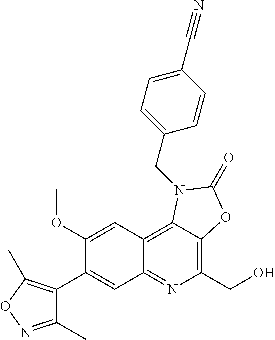 [SMILES: COc1cc2c(cc1-c1c(C)noc1C)nc(CO)c1oc(=O)n(Cc3ccc(C#N)cc3)c12] | 4-[[7-(3,5-Dimethylisoxazol-4-yl)-4- (hydroxymethyl)-8-methoxy-2-oxo-oxazolo[5,4- c]quinolin-1-yl]methyl]benzonitrile |
| 90 | 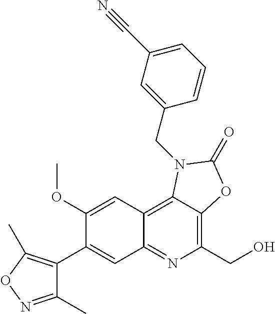 [SMILES: COc1cc2c(cc1-c1c(C)noc1C)nc(CO)c1oc(=O)n(Cc3cccc(C#N)c3)c12] | 3-[[7-(3,5-Dimethylisoxazol-4-yl)-4- (hydroxymethyl)-8-methoxy-2-oxo-oxazolo[5,4- c]quinolin-1-yl]methyl]benzonitrile |
| 91 | 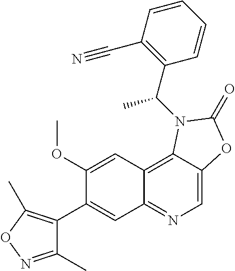 [SMILES: COc1cc2c(cc1-c1c(C)noc1C)ncc1oc(=O)n([C@H](C)c3ccccc3C#N)c12] | 2-[(1R)-1-[7-(3,5-Dimethylisoxazol-4-yl)-8- methoxy-2-oxo-oxazolo[5,4-c]quinolin-1- yl]ethyl]benzonitrile |
| 92 | 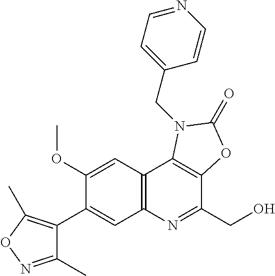 [SMILES: COc1cc2c(cc1-c1c(C)noc1C)nc(CO)c1oc(=O)n(Cc3ccncc3)c12] | 7-(3,5-Dimethylisoxazol-4-yl)-4- (hydroxymethyl)-8-methoxy-1-(4- pyridylmethyl)oxazolo[5,4-c]quinolin-2-one |
| 93 | 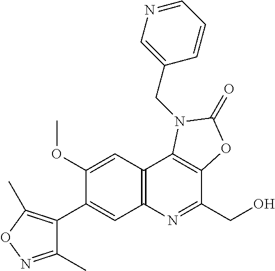 [SMILES: COc1cc2c(cc1-c1c(C)noc1C)nc(CO)c1oc(=O)n(Cc3cccnc3)c12] | 7-(3,5-Dimethylisoxazol-4-yl)-4- (hydroxymethyl)-8-methoxy-1-(3- pyridylmethyl)oxazolo[5,4-c]quinolin-2-one |
| 94 | 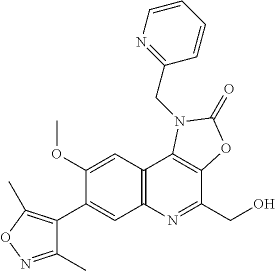 [SMILES: COc1cc2c(cc1-c1c(C)noc1C)nc(CO)c1oc(=O)n(Cc3ccccn3)c12] | 7-(3,5-Dimethylisoxazol-4-yl)-4- (hydroxymethyl)-8-methoxy-1-(2- pyridylmethyl)oxazolo[5,4-c]quinolin-2-one |
| 95 | 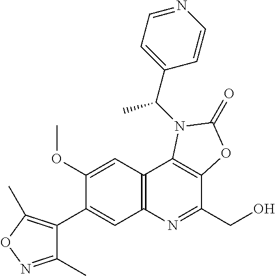 [SMILES: COc1cc2c(cc1-c1c(C)noc1C)nc(CO)c1oc(=O)n([C@H](C)c3ccncc3)c12] | 7-(3,5-Dimethylisoxazol-4-yl)-4- (hydroxymethyl)-8-methoxy-1-[(1R)-1-(4- pyridyl)ethyl]oxazolo[5,4-c]quinolin-2-one |
| 96 | 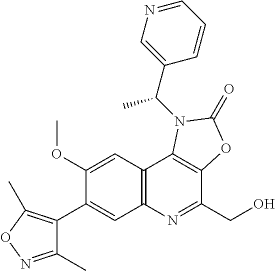 [SMILES: COc1cc2c(cc1-c1c(C)noc1C)nc(CO)c1oc(=O)n([C@H](C)c3cccnc3)c12] | 7-(3,5-Dimethylisoxazol-4-yl)-4- (hydroxymethyl)-8-methoxy-1-[(1R)-1-(3- pyridyl)ethyl]oxazolo[5,4-c]quinolin-2-one |
| 97 | 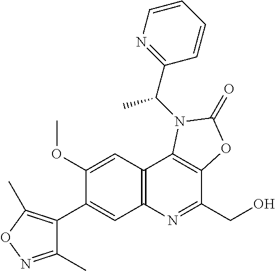 [SMILES: COc1cc2c(cc1-c1c(C)noc1C)nc(CO)c1oc(=O)n([C@H](C)c3ccccn3)c12] | 7-(3,5-Dimethylisoxazol-4-yl)-4- (hydroxymethyl)-8-methoxy-1-[(1R)-1-(2- pyridyl)ethyl]oxazolo[5,4-c]quinolin-2-one |

### Process to Form Compounds of Formulas of the Present Invention

An exemplary process to form particular compounds of formulas of the present invention is shown in Scheme 1.

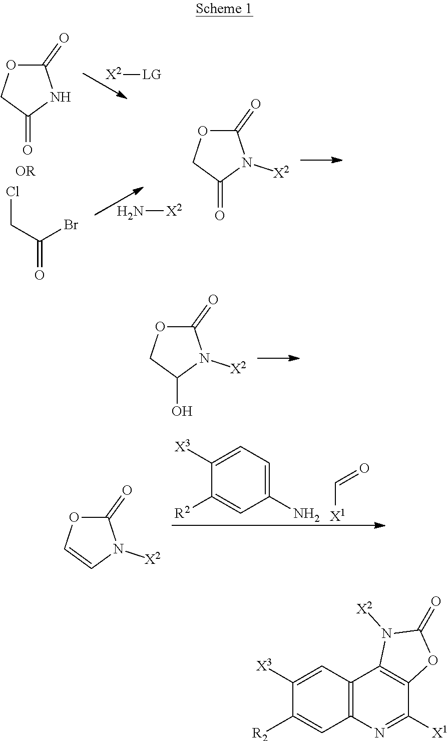

wherein X1, X2, and X3 are previously defined herein and LG is a leaving group.

Another exemplary process to form particular compounds of formulas of the present invention is shown in Scheme 2.

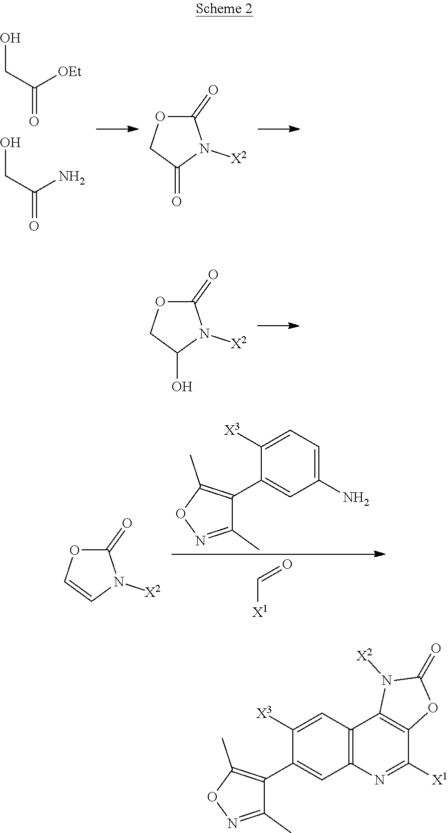

wherein X1, X2, and X3 are previously defined herein.

In certain embodiments, the present invention provides a method of inhibiting a bromodomain-containing protein (such as a BET protein, e.g., BRD2, BRD3, BRD4, and/or BRDT) comprising contacting said bromodomain-containing protein with any compound depicted in the tables herein, or a pharmaceutically acceptable salt or composition thereof.

One embodiment of the present invention provides a method of treating cancer comprising administering to a patient with cancer a therapeutically effective amount of a compound of formula (I) according to the invention or a pharmaceutically acceptable salt thereof, alone or admixed with a pharmaceutically acceptable carrier. Another embodiment of the present invention provides a method of treating cancer comprising administering to a patient with cancer a therapeutically effective amount of a compound of formula (Ia) according to the invention or a pharmaceutically acceptable salt thereof, alone or admixed with a pharmaceutically acceptable carrier. Another embodiment of the present invention provides a method of treating cancer comprising administering to a patient with cancer a therapeutically effective amount of a compound of formula (II) according to the invention or a pharmaceutically acceptable salt thereof, alone or admixed with a pharmaceutically acceptable carrier.

One embodiment of the present invention provides pharmaceutically acceptable preparations comprising a compound of formula (I) and pharmaceutically acceptable excipient. Another embodiment of the present invention provides pharmaceutically acceptable preparations comprising a compound of formula (Ia) and pharmaceutically acceptable excipient. Another embodiment of the present invention provides pharmaceutically acceptable preparations comprising a compound of formula (II) and pharmaceutically acceptable excipient.

One embodiment of the present invention provides a method of treating cancer, wherein said cancer is selected from the group consisting of brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone and thyroid.

One embodiment of the present invention provides composition of a compound or compounds of the present invention in combination with an additional therapeutic agent.

One embodiment of the present invention provides composition of a compound or compounds of formula (I) in combination with an additional therapeutic agent. Another embodiment of the present invention provides composition of a compound or compounds of formula (Ia) in combination with an additional therapeutic agent. Another embodiment of the present invention provides composition of a compound or compounds of formula (II) in combination with an additional therapeutic agent.

One embodiment of the present invention provides a method for inhibiting activity of a bromodomain-containing protein, or a mutant thereof, in a biological sample comprising the step of contacting said biological sample with compound or compounds formula (I). Another embodiment of the present invention provides a method for inhibiting activity of a bromodomain-containing protein, or a mutant thereof, in a biological sample comprising the step of contacting said biological sample with compound or compounds formula (Ia). Another embodiment of the present invention provides a method for inhibiting activity of a bromodomain-containing protein, or a mutant thereof, in a biological sample comprising the step of contacting said biological sample with compound or compounds formula (II).

One embodiment of the present invention provides a method for inhibiting activity of a bromodomain-containing protein, wherein the bromodomain-containing protein is a BET protein.

One embodiment of the present invention provides a method for inhibiting activity of a bromodomain-containing protein, wherein the BET protein is BRD4.

One embodiment of the present invention provides a method for inhibiting activity of a bromodomain-containing protein, or a mutant thereof, activity in a patient comprising the step of administering to said patient a compound or compounds of formula (I). Another embodiment of the present invention provides a method for inhibiting activity of a bromodomain-containing protein, or a mutant thereof, activity in a patient comprising the step of administering to said patient a compound or compounds of formula (Ia). Another embodiment of the present invention provides a method for inhibiting activity of a bromodomain-containing protein, or a mutant thereof, activity in a patient comprising the step of administering to said patient a compound or compounds of formula (II).

One embodiment of the present invention provides a method for treating a bromodomain-containing protein-mediated disorder in a patient in need thereof, comprising the step of administering to said patient a compound or compounds of formula (I). Another embodiment of the present invention provides a method for treating a bromodomain-containing protein-mediated disorder in a patient in need thereof, comprising the step of administering to said patient a compound or compounds of formula (Ia). Another embodiment of the present invention provides a method for treating a bromodomain-containing protein-mediated disorder in a patient in need thereof, comprising the step of administering to said patient a compound or compounds of formula (II).

One embodiment of the present invention provides a method for treating a bromodomain-containing protein-mediated disorder in a patient in need thereof, wherein the bromodomain-containing protein is a BET protein.

One embodiment of the present invention provides a method for treating a bromodomain-containing protein-mediated disorder in a patient in need thereof, wherein the BET protein is BRD4.

One embodiment of the present invention provides a method for treating a bromodomain-containing protein-mediated disorder in a patient in need thereof, wherein the disorder is a proliferative disorder, inflammatory disease, sepsis, autoimmune disease, or viral infection.

One embodiment of the present invention provides a method for treating a bromodomain-containing protein-mediated disorder in a patient in need thereof, wherein the proliferative disorder is cancer.

One embodiment of the present invention provides a method for treating a bromodomain-containing protein-mediated disorder in a patient in need thereof, wherein the cancer is adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, or Wilms' tumor.

One embodiment of the present invention provides a method for treating a bromodomain-containing protein-mediated disorder in a patient in need thereof, wherein the cancer is acute myelognous leukemia or Burkitt's lymphoma.

One embodiment of the present invention provides a method for treating a bromodomain-containing protein-mediated disorder in a patient in need thereof, wherein the inflammatory disease is rheumatoid arthritis, irritable bowel syndrome or psoriasis.

Another embodiment of the present invention provides a method of treating autoimmune and inflammatory diseases or conditions which comprises administering to a subject in need thereof a therapeutically effective amount of a bromodomain inhibitor.

Another embodiment of the present invention provides a method of treating autoimmune and inflammatory diseases or conditions in which the bromodomain inhibitor is a compound that inhibits the binding of BET family bromodomains to acetylated lysine residues.

Another embodiment of the present invention provides a method of treating autoimmune and inflammatory diseases or conditions in which the BET family bromodomain is BRD2, BRD3 or BRD4.

Another embodiment of the present invention provides a method of treating autoimmune and inflammatory diseases or conditions in which the autoimmune and inflammatory diseases or conditions involve an inflammatory response to infections with bacteria, viruses, fungi, parasites or their toxins, as well as viruses.

Another embodiment of the present invention provides a method of treating autoimmune and inflammatory diseases or conditions method in which the autoimmune and inflammatory diseases or conditions are selected from the group consisting of acute lung injury, acute pancreatitis, acute renal failure, ARDS (adult respiratory distress syndrome), burns, coronavirus, encephalitis, endotoxaemia, fulminant hepatitis, herpes simplex, herpes zoster, Herxheimer reactions, malaria and SIRS associated with viral infections such as influenza, meningitis, multi-organ dysfunction syndrome, myelitis, post-surgical syndromes, sarcoidosis, sepsis, sepsis syndrome, septic shock, systemic inflammatory response syndrome (SIRS), toxic shock syndrome.

Another embodiment of the present invention provides a bromodomain inhibitor for use in the treatment of autoimmune and inflammatory diseases or conditions.

Another embodiment of the present invention provides a use of a bromodomain inhibitor in the manufacture of a medicament for the treatment of autoimmune and inflammatory diseases or conditions.

Another embodiment of the present invention provides a pharmaceutical formulation comprising a bromodomain inhibitor and at least one pharmaceutical carrier, wherein the bromodomain inhibitor is present in an amount effective for use in the treatment of autoimmune and inflammatory diseases or conditions.

Another embodiment of the present invention provides a method for identifying compounds for use in treating autoimmune and inflammatory diseases or conditions which comprises the step of determining whether the compound inhibits the binding of a bromodomain with its cognate acetylated protein.

An embodiment of the present invention provides compound(s) for use in treating autoimmune and inflammatory diseases or conditions.

### Uses, Formulation and Administration

(i) Pharmaceutically Acceptable Compositions

According to another embodiment, the present invention provides a method of inhibiting a bromodomain-containing protein (such as a BET protein, e.g., BRD2, BRD3, BRD4, and/or BRDT) using a composition comprising a compound of the invention or a pharmaceutically acceptable derivative thereof and a pharmaceutically acceptable carrier, adjuvant, or vehicle. The amount of a compound of the invention in a provided composition is such that is effective to measurably inhibit one or more bromodomain-containing proteins (such as a BET protein, e.g., BRD2, BRD3, BRD4, and/or BRDT), or a mutant thereof, in a biological sample or in a patient. In certain embodiments, the amount of compound in a provided composition is such that is effective to measurably inhibit one or more bromodomain-containing proteins (such as a BET protein, e.g., BRD2, BRD3, BRD4, and/or BRDT), or a mutant thereof, in a biological sample or in a patient. In certain embodiments, a provided composition is formulated for administration to a patient in need of such composition. In some embodiments, a provided composition is formulated for oral administration to a patient.

The term “patient,” as used herein, means an animal, such as a mammal, such as a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitory active metabolite or residue thereof.

As used herein, the term “inhibitory active metabolite or residue thereof” means that a metabolite or residue thereof is also an inhibitor of one or more bromodomain-containing proteins (such as a BET protein, e.g., BRD2, BRD3, BRD4, and/or BRDT), or a mutant thereof.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Provided compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of provided compounds that may be combined with carrier materials to produce a composition in a single dosage form will vary depending upon the patient to be treated and the particular mode of administration. Provided compositions may be formulate such that a dosage of between 0.01-100 mg/kg body weight/day of the inhibitor can be administered to a patient receiving these compositions.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided compound in the composition will also depend upon the particular compound in the composition.

(ii) Uses of Compounds and Pharmaceutically Acceptable Compositions

Compounds and compositions described herein are generally useful for the inhibition of activity of one or more proteins involved in epigenetic regulation. Thus, in some embodiments, the present invention provides a method of inhibiting one or more proteins involved in epigenetic regulation, such as proteins containing acetyl-lysine recognition motifs, also known as bromodomains (e.g., BET proteins, such as BRD2, BRD3, BRD4, and/or BRDT), by administering a provided compound or composition.

Epigenetics is the study of heritable changes in gene expression caused by mechanisms other than changes in the underlying DNA sequence. Molecular mechanisms that play a role in epigenetic regulation include DNA methylation and chromatin/histone modifications. Chromatin recognition, in particular, is critical in many epigenetic phenomena.

Chromatin, the organized assemblage of nuclear DNA and histone proteins, is the basis for a multitude of vital nuclear processes including regulation of transcription, replication, DNA-damage repair and progression through the cell cycle. A number of factors, such as chromatin-modifying enzymes, have been identified that play an important role in maintaining the dynamic equilibrium of chromatin (Margueron, et al. (2005) Curr. Opin. Genet. Dev. 15:163-176).

Histones are the chief protein components of chromatin. They act as spools around which DNA winds, and they play a role in gene regulation. There are a total of six classes of histones (HI, H2A, H2B, H3, H4, and H5) organized into two super classes: core histones (H2A, H2B, H3, and H4) and linker histones (HI and H5). The basic unit of chromatin is the nucleosome, which consists of about 147 base pairs of DNA wrapped around the histone octamer, consisting of two copies each of the core histones H2A, H2B, H3, and H4 (Luger, et al. (1997) Nature 389:251-260).

Histones, particularly residues of the amino termini of histones H3 and H4 and the amino and carboxyl termini of histones H2A, H2B and HI, are susceptible to a variety of post-translational modifications including acetylation, methylation, phosphorylation, ribosylation sumoylation, ubiquitination, citrullination, deimination, and biotinylation. The core of histones H2A and H3 can also be modified. Histone modifications are integral to diverse biological processes such as gene regulation, DNA repair, and chromosome condensation.

One type of histone modification, lysine acetylation, is recognized by bromodomain-containing proteins. Bromodomain-containing proteins are components of transcription factor complexes and determinants of epigenetic memory (Dey, et al. (2009) Mol. Biol. Cell 20:4899-4909). There are 46 human proteins containing a total of 57 bromodomains discovered to date. One family of bromodomain-containing proteins, BET proteins (BRD2, BRD3, BRD4, and BRDT) have been used to establish proof-of-concept for targeting protein-protein interactions of epigenetic “readers,” as opposed to chromatin-modifying enzymes, or so-called epigenetic “writers” and “erasers” (Filippakopoulos, et al. “Selective Inhibition of BET Bromodomains,” Nature (published online Sep. 24, 2010); Nicodeme, et al. “Suppression of Inflammation by a Synthetic Histone Mimic,” Nature (published online Nov. 10, 2010)).

Examples of proteins inhibited by the compounds and compositions described herein and against which the methods described herein are useful include bromodomain-containing proteins, such as BET proteins, such as BRD2, BRD3, BRD4, and/or BRDT, or an isoform or mutant thereof.

The activity of a provided compound, or composition thereof, as an inhibitor of a bromodomain-containing protein, such as a BET protein, such as BRD2, BRD3, BRD4, and/or BRDT, or an isoform or mutant thereof, may be assayed in vitro, in vivo, or in a cell line. In vitro assays include assays that determine inhibition of bromodomain-containing proteins, such as BET proteins, such as BRD2, BRD3, BRD4, and/or BRDT, or a mutant thereof. Alternatively, inhibitor binding may be determined by running a competition experiment where a provided compound is incubated with a bromodomain-containing protein, such as a BET protein, such as BRD2, BRD3, BRD4, and/or BRDT bound to known ligands, labeled or unlabeled. Detailed conditions for assaying a provided compound as an inhibitor of a bromodomain-containing protein, such as a BET protein, such as BRD2, BRD3, BRD4, and/or BRDT or a mutant thereof.

The invention provides for a method of treating a subject with a MYC-dependent cancer, comprising: identifying a subject in need of treatment; administering to the subject a BET inhibitor; determining at least one of MYC mRNA expression, MYC protein expression and tumor mass, and wherein following administration, there is a decrease in at least one of MYC mRNA expression, MYC protein expression and tumor mass, thereby treating the disease.

In one embodiment, the identification step comprises determining whether the subject has at least one of a MYC translocation, a genetic rearrangement of MYC, MYC amplification, MYC over-expression and at least one cellular function that facilitates cellular and/or tumor growth and is altered upon reduction of MYC mRNA or protein expression.

The invention also provides for a method of treating a subject with a MYC-dependent cancer, comprising: determining at least one of MYC mRNA expression, MYC protein expression and tumor mass; administering to the subject a BET inhibitor; and comparing at least one of MYC mRNA expression, MYC protein expression and tumor mass in the subject before and after administration of the BET inhibitor.

The invention also provides a method of treating a subject with a MYC-dependent cancer, comprising: administering to the subject a BET inhibitor that is identified as capable of decreasing at least one of MYC mRNA expression, MYC protein expression and tumor mass; and determining at least one of MYC mRNA expression, MYC protein expression and tumor mass; wherein following the administration, there is a decrease in at least one of MYC mRNA expression, MYC protein expression and tumor mass, thereby treating the disease.

The invention also provides for a method of treating a subject with a disease, comprising: administering a BET inhibitor that is identified as capable of decreasing at least one of MYC mRNA expression, MYC protein expression and tumor mass, wherein following the administration, there is a decrease in at least one of MYC mRNA expression, MYC protein expression and tumor mass, thereby treating the disease.

Acetylated histone recognition and bromodomain-containing proteins (such as BET proteins) have been implicated in proliferative disease. BRD4 knockout mice die shortly after implantation and are compromised in their ability to maintain an inner cell mass, and heterozygotes display pre- and postnatal growth defects associated with reduced proliferation rates. BRD4 regulates genes expressed during M/Gl, including growth-associated genes, and remains bound to chromatin throughout the cell cycle (Dey, et al. (2009) Mol. Biol. Cell 20:4899-4909). BRD4 also physically associates with Mediator and P-TEFb (CDK9/cyclin Tl) to facilitate transcriptional elongation (Yang, et al. (2005) Oncogene 24:1653-1662; Yang, et al. (2005) Mol. Cell 19:535-545). CDK9 is a validated target in chronic lymphocytic leukemia (CLL), and is linked to c-MYC-dependent transcription (Phelps, et al. Blood 113:2637-2645; Rahl, et al. (2010) Cell 141:432-445).

BRD4 is translocated to the NUT protein in patients with lethal midline carcinoma, an aggressive form of human squamous carcinoma (French, et al. (2001) Am. J. Pathol. 159:1987-1992; French, et al. (2003) Cancer Res. 63:304-307). In vitro analysis with RNAi supports a causal role for BRD4 in this recurrent t(15;19) chromosomal translocation. Pharmacologic inhibition of the BRD4 bromodomains results in growth arrest/differentiation of BRD4-NUT cell lines in vitro and in vivo (Filippakopoulos, et al. “Selective Inhibition of BET Bromodomains,” Nature (published online Sep. 24, 2010)).

Bromodomain-containing proteins (such as BET proteins) have also been implicated in inflammatory diseases. BET proteins {e.g., BRD2, BRD3, BRD4, and BRDT) regulate assembly of histone acetylation-dependent chromatin complexes that control inflammatory gene expression (Hargreaves, et al. (2009) Cell 138:129-145; LeRoy, et al. (2008) Mol. Cell 30:51-60; Jang, et al. (2005) Mol. Cell 19:523-534; Yang, et al. (2005) Mol. Cell 19:535-545). Key inflammatory genes (secondary response genes) are down-regulated upon bromodomain inhibition of the BET subfamily, and non-responsive genes (primary response genes) are poised for transcription. BET bromodomain inhibition protects against LPS-induced endotoxic shock and bacteria-induced sepsis in vivo (Nicodeme, et al. “Suppression of Inflammation by a Synthetic Histone Mimic,” Nature (published online Nov. 10, 2010)).

Bromodomain-containing proteins (such as BET proteins) also play a role in viral disease. For example, BRD4 is implicated in human papilloma virus (HPV). In the primary phase of HPV infection of basal epithelia, the viral genome is maintained in an extra-chromosomal episome. In some strains of HPV, BRD4 binding to the HPV E2 protein functions to tether the viral genome to chromosomes. E2 is critical for both the repression of E6/E7 and to activation of HPV viral genes. Disruption of BRD4 or the BRD4-E2 interaction blocks E2-dependent gene activation. BRD4 also functions to tether other classes of viral genomes to host chromatin (e.g., Herpesvirus, Epstein-Barr virus).

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In certain embodiments, a provided compound inhibits one or more of BRD2, BRD3, BRD4, BRDT, and/or another member of the bromodomain-containing proteins, or a mutant thereof. In some embodiments, a provided compound inhibits two or more of BRD2, BRD3, BRD4, BRDT, and/or another member of the bromodomain-containing proteins, or a mutant thereof. Provided compounds are inhibitors of one of more of the bromodomain-containing proteins, such as BRD2, BRD3, BRD4, and/or BRDT and are therefore useful for treating one or more disorders associated with activity of one or more of the bromodomain-containing proteins, such as BRD2, BRD3, BRD4, and/or BRDT. Thus, in certain embodiments, the present invention provides a method for treating an bromodomain-containing protein-mediated disorder, such as a BET-mediated, a BRD2-mediated, a BRD3-mediated, a BRD4-mediated disorder, and/or a BRDT-mediated disorder comprising the step of inhibiting a bromodomain-containing protein, such as a BET protein, such as BRD2, BRD3, BRD4, and/or BRDT, or a mutant thereof, by administering to a patient in need thereof a provided compound, or a pharmaceutically acceptable composition thereof.

As used herein, the terms “bromodomain-containing protein-mediated”, “BET-mediated”, “BRD2-mediated”, “BRD3-mediated”, “BRD4-mediated”, and/or “BRDT-mediated” disorders or conditions means any disease or other deleterious condition in which one or more of the bromodomain-containing proteins, such as BET proteins, such as BRD2, BRD3, BRD4 and/or BRDT, or a mutant thereof, are known to play a role.

Accordingly, another embodiment of the present invention relates to treating or lessening the severity of one or more diseases in which one or more of the bromodomain-containing proteins, such as BET proteins, such as BRD2, BRD3, BRD4, and/or BRDT, or a mutant thereof, are known to play a role.

Diseases and conditions treatable according to the methods of this invention include, but are not limited to, cancer and other proliferative disorders, inflammatory diseases, sepsis, autoimmune disease, and viral infection. Thus one aspect is a method of treating a subject having a disease, disorder, or symptom thereof the method including administration of a compound or composition herein to the subject. In one embodiment, a human patient is treated with a compound of the invention and a pharmaceutically acceptable carrier, adjuvant, or vehicle, wherein said compound is present in an amount to measurably inhibit bromodomain-containing protein activity (such as BET protein, e.g., BRD2, BRD3, BRD4, and/or BRDT) in the patient.

The invention further relates to a method for treating or ameliorating cancer or another proliferative disorder by administration of an effective amount of a compound according to this invention to a mammal, in particular a human in need of such treatment. In some aspects of the invention, the disease to be treated by the methods of the present invention is cancer. Examples of cancers treated using the compounds and methods described herein include, but are not limited to, acinic cell carcinoma, acoustic neuroma, acral lentiginous melanoma, acrospiroma, acute eosinophilic leukemia, acute erythroid leukemia, acute lymphoblastic leukemia, acute lymphocytic leukemia, acute megakaryoblastic leukemia, acute monocytic leukemia, acute myelogenous leukemia, acute myelognous leukemia, acute promyelocytic leukemia, adrenal cancer, adenocarcinoma, adenoid cystic carcinoma, adenoma, adenomatoid odontogenic tumor, adenosquamous carcinoma, adipose tissue neoplasm, adrenal cancer, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, Bannayan-Zonana syndrome, basal cell carcinoma, B-cell chronic lymphocytic leukemia, B-cell lymphoma, B-cell prolymphocytic leukemia, biliary tract cancer, bladder, bladder cancer, blastoma, bone cancer, brain (gliomas), brain cancer, breast, breast cancer, Brenner tumor, Brown tumor, Burkitt's lymphoma, Burkitt's lymphoma, carcinoma, carcinoma in situ, carcinosarcoma, cartilage tumor, cementoma, cervical cancer, chondroma, chordoma, choriocarcinoma, choroid plexus papilloma, chronic lymphocytic leukemia, clear-cell sarcoma of the kidney, colon, colorectal cancer, Cowden disease, craniopharyngioma, cutaneous T-cell lymphoma, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, ependymoma, esophageal cancer, Ewing's sarcoma, fetus in fetu, fibroma, fibrosarcoma, follicular lymphoma, follicular thyroid cancer, gallbladder cancer, ganglioneuroma, gastric, gastric cancer, gastrointestinal cancer, germ cell tumor, gestational choriocarcinoma, giant cell fibroblastoma, giant cell tumor of bone and thyroid, giant cell tumor of the bone, glial tumor, glioblastoma multiforme, glioblastomas, glioma, gliomatosis cerebri, glucagonoma, gonadoblastoma, granulosa cell tumor, gynandroblastoma, hairy cell leukemia, head and neck, head and neck cancer, hemangioblastoma, hemangiopericytoma, hematological malignancy, hepatoblastoma, hepatosplenic T-cell lymphoma, Hodgkin's lymphoma, inflammatory breast cancer, intestinal cancer, invasive lobular carcinoma, kidney, kidney cancer, laryngeal cancer, lentigo maligna, lethal midline carcinoma, leukemia, leukemias, leydig cell tumor, Lhermitte-Duclos disease, liposarcoma, liver, liver cancer, lung, lung cancer, lymphangio sarcoma, lymphangioma, lymphoepithelioma, lymphoma, lymphomas, malignant fibrous histiocytoma, malignant peripheral nerve sheath tumor, malignant triton tumor, MALT lymphoma, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myeloid sarcoma, myxoid liposarcoma, myxoma, myxosarcoma, nasopharyngeal carcinoma, neurinoma, neuroblastoma, neurofibroma, neuroma, nodular melanoma, non-Hodgkin's lymphoma, non-small cell lung cancer, ocular cancer, oligoastrocytoma, oligodendroglioma, oncocytoma, optic nerve sheath meningioma, optic nerve tumor, oral cancer, osteosarcoma, ovarian, ovarian cancer, Pancoast tumor, pancreatic, pancreatic cancer, papillary thyroid cancer, paraganglioma, pharyngeal cancer, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate, prostate cancer, pseudomyxoma peritonei, rectal cancer, renal, renal cell carcinoma, renal medullary carcinoma, retinoblastoma, rhabdomyoma, Rhabdomyosarcoma, Richter's transformation, sarcoma, Schwannomatosis, seminoma, Sertoli cell tumor, sex cord-gonadal stromal tumor, Sezary's disease, signet ring cell carcinoma, skin cancer, small blue round cell tumors, small cell carcinoma, small cell lung cancer, small intestine cancer, soft tissue sarcoma, somatostatinoma, soot wart, spinal tumor, splenic marginal zone lymphoma, squamous carcinoma, squamous cell carcinoma, stomach cancer, synovial sarcoma, T-cell lymphoma, testicular cancer, thecoma, throat cancer, thyroid cancer, transitional cell carcinoma, urachal cancer, urogenital cancer, urothelial carcinoma, uterine cancer, uveal melanoma, vaginal cancer, verrucous carcinoma, visual pathway glioma, vulvar cancer, Waldenstrom's macroglobulinemia, Warthin's tumor, and Wilm's tumor.

In some embodiments, the present invention provides a method of treating other conditions. Such other conditions include, but are not limited to, acne, acute inflammatory responses (such as acute respiratory distress syndrome and ischemia/reperfusion injury, glioblastoma, Graves' disease, HIV, HPV, inflammatory disease, keloids and related scarring, lung cancer, meningitis (bacterial and viral), multiple sclerosis, neoplasm, neuroblastoma, pancreatic cancer, scleroderma, skin cancer, toxic shock, viral infections, viral infections and diseases.

In some embodiments, the present invention provides a method of treating a benign proliferative disorder. Such benign proliferative disorders include, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, prolactinoma, pseudotumor cerebri, pyogenic granuloma, and juvenile polyposis syndrome.

The invention further relates to a method for treating infectious and noninfectious inflammatory events and autoimmune and other inflammatory diseases by administration of an effective amount of a provided compound to a mammal, in particular a human in need of such treatment. Examples of autoimmune and inflammatory diseases, disorders, and syndromes treated using the compounds and methods described herein include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholecystitis, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, Type I diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleracierma, mycosis fungoides, acute inflammatory responses (such as acute respiratory distress syndrome and ischemia/reperfusion injury), and Graves' disease. Other examples of infectious and noninfectious inflammatory events, autoimmune and other inflammatory diseases include, but are not limited to, Addison's disease, agammaglobulinemia, allergic rhinitis, allergy, Alzheimer's disease, appendicitis, asthma, atherosclerosis, atopic dermatitis, autoimmune alopecia, autoimmune hemolytic and thrombocytopenic states, autoimmune hypopituitarism, autoimmune polyglandular disease (also known as autoimmune polyglandular syndrome), Behcet's disease, cholecystitis, chronic idiopathic thrombocytopenic purpura, chronic obstructive pulmonary disease (COPD), Crohn's disease, degenerative joint disease, dermatitis, dermatomyositis, encephalitis, enteritis, gastritis, gingivitis, glomerulonephritis, Goodpasture's syndrome, Guillain-Barre syndrome, Hashimoto's thyroiditis, hepatitis, hyperacute rejection of transplanted organs, inflammatory pelvic disease, irritable bowel syndrome, juvenile arthritis, meningitis, multiple sclerosis, myasthenia gravis, mycosis fungoides, myocarditis, myositis, nephritis, osteoarthritis, osteomyelitis, pancreatitis, Parkinson's disease, pernicious anemia, pneumonitis, psoriasis, psoriatic arthritis, rheumatoid arthritis, scleracierma, scleroderma, septic shock, sinusitis, Sjogren's disease, skin sunburn, systemic lupus erythematosus (SLE), tissue graft rejection, Type I diabetes, ulcerative colitis, urethritis, vasculitis, vitiligo, and Waldenstrom macroglobulinemia.

In some embodiments, the present invention provides a method of treating systemic inflammatory response syndromes such as LPS-induced endotoxic shock and/or bacteria-induced sepsis by administration of an effective amount of a provided compound to a mammal, in particular a human in need of such treatment.

The invention further relates to a method for treating viral infections and diseases by administration of an effective amount of a provided compound to a mammal, in particular a human in need of such treatment. Examples of viral infections and diseases treated using the compounds and methods described herein include episome-based DNA viruses including, but not limited to, human papillomavirus, Herpesvirus, Epstein-Barr virus, human immunodeficiency virus, hepatis B virus, and hepatitis C virus.

The invention further provides a method of treating a subject, such as a human, suffering from one of the abovementioned conditions, illnesses, disorders or diseases. The method comprises administering a therapeutically effective amount of one or more provided compounds, which function by inhibiting a bromodomain and, in general, by modulating gene expression, to induce various cellular effects, in particular induction or repression of gene expression, arresting cell proliferation, inducing cell differentiation and/or inducing apoptosis, to a subject in need of such treatment.

The invention further provides a therapeutic method of modulating protein methylation, gene expression, cell proliferation, cell differentiation and/or apoptosis in vivo in diseases mentioned above, in particular cancer, inflammatory disease, and/or viral disease comprising administering to a subject in need of such therapy a pharmacologically active and therapeutically effective amount of one or more provided compounds.

The invention further provides a method of regulating endogenous or heterologous promoter activity by contacting a cell with a provided compound.

In certain embodiments, the invention provides a method of treating a disorder (as described above) in a subject, comprising administering to the subject identified as in need thereof, a compound of the invention. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

A method of assessing the efficacy of a treatment in a subject includes determining the pre-treatment extent of a disorder by methods well known in the art (e.g., determining tumor size or screening for tumor markers where the cell proliferative disorder is cancer) and then administering a therapeutically effective amount of a compound of the invention, to the subject. After an appropriate period of time after the administration of the compound (e.g., 1 day, 1 week, 2 weeks, one month, six months), the extent of the disorder is determined again. The modulation (e.g., decrease) of the extent or invasiveness of the disorder indicates efficacy of the treatment. The extent or invasiveness of the disorder may be determined periodically throughout treatment. For example, the extent or invasiveness of the disorder may be checked every few hours, days or weeks to assess the further efficacy of the treatment. A decrease in extent or invasiveness of the disorder indicates that the treatment is efficacious. The method described may be used to screen or select patients that may benefit from treatment with a compound of the invention.

The invention further relates to the use of provided compounds for the production of pharmaceutical compositions which are employed for the treatment and/or prophylaxis and/or amelioration of the diseases, disorders, illnesses and/or conditions as mentioned herein.

The invention further relates to the use of provided compounds for the production of pharmaceutical compositions which are employed for the treatment and/or prophylaxis of diseases and/or disorders responsive or sensitive to the inhibition of bromodomain-containing proteins, particularly those diseases mentioned above, such as e.g. cancer, inflammatory disease, viral disease.

Another object of the present invention is the use of a compound as described herein (e.g., of any formulae herein) in the manufacture of a medicament for use in the treatment of a disorder or disease herein. Another object of the present invention is the use of a compound as described herein (e.g., of any formulae herein) for use in the treatment of a disorder or disease herein.

Compounds or compositions described herein may be administered using any amount and any route of administration effective for treating or lessening the severity of cancer or other proliferative disorder. The exact amount required will vary from subject to subject, depending on the species, age, and general condition of the subject, the severity of the infection, the particular agent, its mode of administration, and the like. Provided compounds are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, provided compounds may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

According to some embodiments, the invention relates to a method of inhibiting bromodomain-containing proteins in a biological sample comprising the step of contacting said biological sample with a provided compound, or a composition thereof.

According to some embodiments, the invention relates to a method of inhibiting a bromodomain-containing protein, such as a BET protein, such as BRD2, BRD3, BRD4 and/or BRDT, or a mutant thereof, activity in a biological sample comprising the step of contacting said biological sample with a provided compound, or a composition thereof.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition of activity of an protein, e.g., a bromodomain-containing protein, such as a BET protein, such as BRD2, BRD3, BRD4 and/or BRDT, or a mutant thereof, in a biological sample is useful for a variety of purposes that are known to one of skill in the art. Examples of such purposes include, but are not limited to, blood transfusion, organ-transplantation, biological specimen storage, and biological assays.

According to another embodiment, the invention relates to a method of inhibiting activity of one or more bromodomain-containing protein, such as a BET protein, such as BRD2, BRD3, BRD4, and/or BRDT, or a mutant thereof, in a patient comprising the step of administering to said patient a provided compound, or a composition comprising said compound. In certain embodiments, the present invention provides a method for treating a disorder mediated by one or more bromodomain-containing proteins, such as a BET protein, such as BRD2, BRD3, BRD4, and/or BRDT, or a mutant thereof, in a patient in need thereof, comprising the step of administering to said patient a provided compound or pharmaceutically acceptable composition thereof. Such disorders are described in detail herein.

Depending upon the particular condition, or disease, to be treated, additional therapeutic agents that are normally administered to treat that condition may also be present in the compositions of this disclosure or administered separately as a part of a dosage regimen. As used herein, additional therapeutic agents that are normally administered to treat a particular disease, or condition, are known as “appropriate for the disease, or condition, being treated.”

In some embodiments, the additional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

Other therapies, chemotherapeutic agents, or other anti-proliferative agents may be combined with a provided compound to treat proliferative diseases and cancer. Examples of therapies or anticancer agents that may be used in combination with compounds of formula (I) or formula (Ia) include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effects (e.g., an antiemetic), and any other approved chemotherapeutic drug.

A provided compound may also be used to advantage in combination with one or more antiproliferative compounds. Such antiproliferative compounds include an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carotenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Exemplary aromatase inhibitors include steroids, such as atamestane, exemestane and formestane, and non-steroids, such as aminoglutethimide, rogletimide, pyridoglutethimide, trilostane, testolactone, ketoconazole, vorozole, fadrozole, anastrozole and letrozole.

Exemplary anti-estrogens include tamoxifen, fulvestrant, raloxifene and raloxifene hydrochloride. Anti-androgens include, but are not limited to, bicalutamide. Gonadorelin agonists include, but are not limited to, abarelix, goserelin and goserelin acetate.

Exemplary topoisomerase I inhibitors include topotecan, gimatecan, irinotecan, camptothecin and its analogues, 9-nitrocamptothecin and the macromolecular camptothecin conjugate PNU-166148. Topoisomerase II inhibitors include, but are not limited to, the anthracyclines such as doxorubicin, daunorubicin, epirubicin, idarubicin and nemorubicin, the anthraquinones mitoxantrone and losoxantrone, and the podophillotoxins etoposide and teniposide.

Exemplary microtubule active agents include microtubule stabilizing, microtubule destabilizing compounds and microtubulin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; colchicine and epothilones and derivatives thereof.

Exemplary alkylating agents include cyclophosphamide, ifosfamide, melphalan or nitrosoureas such as carmustine and lomustine.

Exemplary cyclooxygenase inhibitors include Cox-2 inhibitors, 5-alkyl substituted 2-arylaminophenylacetic acid and derivatives, such as celecoxib, rofecoxib, etoricoxib, valdecoxib or a 5-alkyl-2-arylaminophenylacetic acid, such as lumiracoxib.

Exemplary matrix metalloproteinase inhibitors (“MMP inhibitors”) include collagen peptidomimetic and non-peptidomimetic inhibitors, tetracycline derivatives, batimastat, marimastat, prinomastat, metastat, BMS-279251, BAY 12-9566, TAA211, MMI270B, and AAJ996.

Exemplary mTOR inhibitors include compounds that inhibit the mammalian target of rapamycin (mTOR) and possess antiproliferative activity such as sirolimus, everolimus, CCI-779, and ABT578.

Exemplary antimetabolites include 5-fluorouracil (5-FU), capecitabine, gemcitabine, DNA demethylating compounds, such as 5-azacytidine and decitabine, methotrexate and edatrexate, and folic acid antagonists such as pemetrexed.

Exemplary platin compounds include carboplatin, cis-platin, cisplatinum, and oxaliplatin.

Exemplary methionine aminopeptidase inhibitors include bengamide or a derivative thereof and PPI-2458.

Exemplary bisphosphonates include etidronic acid, clodronic acid, tiludronic acid, pamidronic acid, alendronic acid, ibandronic acid, risedronic acid and zoledronic acid.

Exemplary antiproliferative antibodies include trastuzumab, trastuzumab-DMl, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary heparanase inhibitors include compounds that target, decrease or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras; for example, a farnesyl transferase inhibitor such as L-744832, DK8G557, tipifarnib, and lonafarnib.

Exemplary telomerase inhibitors include compounds that target, decrease or inhibit the activity of telomerase, such as compounds which inhibit the telomerase receptor, such as telomestatin.

Exemplary proteasome inhibitors include compounds that target, decrease or inhibit the activity of the proteasome including, but not limited to, bortezomib.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c) and busulfan; and ALK inhibitors, which are compounds which target, decrease or inhibit anaplastic lymphoma kinase.

Exemplary Flt-3 inhibitors include PKC412, midostaurin, a staurosporine derivative, SU11248 and MLN518.

Exemplary HSP90 inhibitors include compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing or inhibiting the activity of the platelet-derived growth factor-receptors (PDGFR), such as a compound which targets, decreases, or inhibits the activity of PDGFR, such as an N-phenyl-2-pyrimidine-amine derivatives, such as imatinib, SU101, SU6668 and GFB-111; b) a compound targeting, decreasing or inhibiting the activity of the fibroblast growth factor-receptors (FGFR); c) a compound targeting, decreasing or inhibiting the activity of the insulin-like growth factor receptor I (IGF-IR), such as a compound which targets, decreases, or inhibits the activity of IGF-IR; d) a compound targeting, decreasing or inhibiting the activity of the Trk receptor tyrosine kinase family, or ephrin B4 inhibitors; e) a compound targeting, decreasing or inhibiting the activity of the Axl receptor tyrosine kinase family; f) a compound targeting, decreasing or inhibiting the activity of the Ret receptor tyrosine kinase; g) a compound targeting, decreasing or inhibiting the activity of the Kit/SCFR receptor tyrosine kinase, such as imatinib; h) a compound targeting, decreasing or inhibiting the activity of the c-Kit receptor tyrosine kinases, such as imatinib; i) a compound targeting, decreasing or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; ISIS 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, C1-1033, EKB-569, GW-2016, antibodies E1.1, E2.4, E2.5, E6.2, E6.4, E2.1 1, E6.3 and E7.6.3, and 7H-pyrrolo-[2,3-d]pyrimidine derivatives; and m) a compound targeting, decreasing or inhibiting the activity of the c-Met receptor.

Exemplary compounds that target, decrease or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g. thalidomide and TNP-470.

Additional exemplary chemotherapeutic compounds, one or more of which may be used in combination with provided compounds, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives,I-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugen; FLT-4 inhibitors, FLT-3 inhibitors, VEGFR-2 IgGI antibody, RPI 4610, bevacizumab, porfimer sodium, anecortave, triamcinolone, hydrocortisone, 11α-epihydrocotisol, cortexolone, 17α-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA or siRNA, or a miscellaneous compound or compound with other or unknown mechanism of action.

For a more comprehensive discussion of updated cancer therapies see: The Merck Manual, 17^{th} Ed. 1999. See also the National Cancer Institute (CNI) website (www.nci.nih.gov) and the Food and Drug Administration (FDA) website for a list of the FDA approved oncology drugs.

Other examples of additional therapeutic agents, one or more of which a provided compound may also be combined with include: a treatment for Alzheimer's Disease such as donepezil and rivastigmine; a treatment for Parkinson's Disease such as L-DOPA/carbidopa, entacapone, ropinirole, pramipexole, bromocriptine, pergolide, trihexyphenidyl, and amantadine; an agent for treating multiple sclerosis (MS) such as beta interferon {e.g., Avonex® and Rebif®), glatiramer acetate, and mitoxantrone; a treatment for asthma such as albuterol and montelukast; an agent for treating schizophrenia such as zyprexa, risperdal, seroquel, and haloperidol; an anti-inflammatory agent such as a corticosteroid, a TNF blocker, IL-1 RA, azathioprine, cyclophosphamide, and sulfasalazine; an immunomodulatory agent, including immunosuppressive agents, such as cyclosporin, tacrolimus, rapamycin, mycophenolate mofetil, an interferon, a corticosteroid, cyclophosphamide, azathioprine, and sulfasalazine; a neurotrophic factor such as an acetylcholinesterase inhibitor, an MAO inhibitor, an interferon, an anti-convulsant, an ion channel blocker, riluzole, or an anti-Parkinson's agent; an agent for treating cardiovascular disease such as a beta-blocker, an ACE inhibitor, a diuretic, a nitrate, a calcium channel blocker, or a statin; an agent for treating liver disease such as a corticosteroid, cholestyramine, an interferon, and an anti-viral agent; an agent for treating blood disorders such as a corticosteroid, an anti-leukemic agent, or a growth factor; or an agent for treating immunodeficiency disorders such as gamma globulin.

The above-mentioned compounds, one or more of which can be used in combination with a provided compound, can be prepared and administered as described in the art.

Provided compounds can be administered alone or in combination with one or more other therapeutic compounds, possible combination therapy taking the form of fixed combinations or the administration of a provided compound and one or more other therapeutic compounds being staggered or given independently of one another, or the combined administration of fixed combinations and one or more other therapeutic compounds. Provided compounds can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Such additional agents may be administered separately from a composition containing a provided compound, as part of a multiple dosage regimen. Alternatively, those agents may be part of a single dosage form, mixed together with a provided compound in a single composition. If administered as part of a multiple dosage regimen, the two active agents may be submitted simultaneously, sequentially or within a period of time from one another normally within five hours from one another.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily inhibitory dose of the compounds of this invention administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In one embodiment, treatment regimens according to the present invention comprise administration to a patient in need of such treatment from about 10 mg to about 1000 mg of the compound(s) of this invention per day in single or multiple doses.

As used herein, the term “combination,” “combined,” and related terms refers to the simultaneous or sequential administration of therapeutic agents in accordance with this invention. For example, a provided compound may be administered with another therapeutic agent simultaneously or sequentially in separate unit dosage forms or together in a single unit dosage form. Accordingly, an embodiment of the invention provides a single unit dosage form comprising a provided compound, an additional therapeutic agent, and a pharmaceutically acceptable carrier, adjuvant, or vehicle for use in the methods of the invention.

The amount of both, a provided compound and additional therapeutic agent (in those compositions which comprise an additional therapeutic agent as described above) that may be combined with the carrier materials to produce a single dosage form will vary depending upon the host treated and the particular mode of administration. Preferably, compositions should be formulated such that a dosage of between 0.01-100 mg/kg body weight/day of a provided compound can be administered.

In those compositions which comprise an additional therapeutic agent, that additional therapeutic agent and the provided compound may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Provided compounds, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a provided compound. Implantable devices coated with a compound of this invention are another embodiment of the present invention.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

In another aspect, the invention provides a method of method of synthesizing a compound of formula (I). In another aspect, the invention provides a method of method of synthesizing a compound of formula (Ia). In another aspect, the invention provides a method of method of synthesizing a compound of formula (II). Another embodiment is a method of making a compound of any of the formulae herein using any one, or combination of, reactions delineated herein. The method can include the use of one or more intermediates or chemical reagents delineated herein.

### EXAMPLES

Whereas the examples may show a single stereoisomeric form, all stereoisomers, and atropoisomers are contemplated. Additionally, all isotopic substitutions (or enrichments), such as deuterium or tritium in place of normal abundance hydrogen, 13C or 14C in place of normal abundance carbon, and 18F in place of normal abundance fluorine, are contemplated.

NMR spectra were recorded on a Bruker 300 MHz and 500 MHz spectrometer; chemical shifts were recorded in parts per million downfield from Me₄Si. Mass spectra were recorded on a Waters Micromass ZQ, using electrospray ionization (ESI). Flash column chromatography was performed with RediSep silica gel columns on an ISCO Combi-Flash. HPLC purifications were performed on a Gilson HPLC with a Phenomenex Gemini column, C18, 150:30 mm, 5 micron, eluting at 40 mL/min with mixtures of MeOH and water containing 0.1% (NH₄)₂CO₃ (high pH), or mixtures of MeCN and water containing 0.1% formic acid (low pH) Anhydrous solvents were purchased from Sigma-Aldrich and stored on 4 Å molecular sieves. Reactions were monitored by TLC analysis or by LC-MS (Column: Phenomonex Polar, Gradient: 10-95% B, Flow rate: 1.75 mL/min, Column temperature: 40° C., Mobile phase: A—0.1% (NH₄)₂CO₃ in H₂O, B—MeOH). Reagents were purchased from commercial sources and used without purification, unless stated otherwise.

### Intermediate Step 1 Toward Example 1

### Oxazolidine-2,4-dione

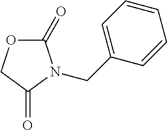 [SMILES: O=C1COC(=O)N1Cc1ccccc1]

A solution of glycolamide (2.10 g, 28 mmol), diethylcarbonate (4.06 mL, 34.0 mmol), and KOtBu (3.14 g, 28 mmol) in MeOH (30 mL) was heated to 75° C. for 18 h under N₂ gas. The mixture was cooled to rt and concentrated under reduced pressure. The residue was diluted in a mixture of brine (100 mL) and 1M HCl (100 mL), and the aq phase was extracted with EtOAc (3×50 mL). The combined organic phases were dried over MgSO₄, filtered, and concentrated under reduced pressure to provide oxazolidine-2,4-dione as a solid (1.56 g, 55%). ¹H NMR (300 MHz, DMSO) δ 11.8 (s, 1H), 4.75 (s, 2H). A solution of the solid from above (500 mg, 5.00 mmol), benzyl chloride (0.569 mL, 5.00 mmol), and TEA (0.69 mL, 5.00 mmol) in CHCl₃ (5.00 mL) was stirred at 60° C. for 48 hours. EtOAc (50.0 mL) and 1M aq HCl (50.0 mL) were added. The aq phase was extracted with CHCl₃ (3×50 mL), and the combined organic phases were washed with brine (50 mL), dried over MgSO₄, filtered, and concentrated under reduced pressure to provide the title compound as a solid (0.894 g, 95%) ¹H NMR (300 MHz, CDCl₃) δ 7.44-7.28 (m, 5H), 4.68 (s, 2H), 4.67 (s, 3H).

### Intermediate Step 2 Toward Example 1

### 3-Benzyl-4-hydroxy-oxazolidin-2-one

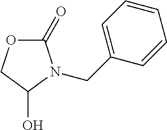 [SMILES: O=C1OCC(O)N1Cc1ccccc1]

A solution of 3-benzyloxazolidine-2,4-dione (894 mg, 5.00 mmol) in MeOH (20.0 mL) was cooled to 0° C., and NaBH₄ (796 mg, 21 mmol) was carefully added in portions. The mixture was warmed to rt for 30 m, and acetone (5 mL) was slowly added. The mixture was concentrated under reduced pressure and diluted in water (50 mL) and CHCl₃ (50 mL). The aq phase was extracted with CHCl₃ (3×50 mL), and the combined organic phases were washed with brine, dried over MgSO₄, filtered, and concentrated under reduced pressure to provide the title compound as a solid (0.687 g, 76%).

### Intermediate Step 3 Toward Example 1

### 3-Benzyloxazol-2-one

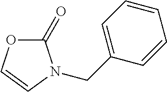 [SMILES: O=c1occn1Cc1ccccc1]

A mixture of 3-benzyl-4-hydroxy-oxazolidin-2-one (687 mg, 4.00 mmol) and TEA (0.992 mL, 7.00 mL) in DCM (10.0 mL) was cooled to 0° C. MsCl (0.413 mL, 5.00 mmol) was added, and the mixture was warmed to rt and stirred for 12 h. The mixture was diluted with water (50 mL), and the aq phase was extracted with CHCl₃ (3×50 mL). The combined organic phases were washed with 0.5M HCl (100 mL) and brine (100 mL). The organic phases were dried over MgSO₄, filtered, and concentrated under reduced pressure to provide the title compound as a solid (0.62 g, 99.5%). ¹H NMR (300 MHz, CDCl₃) δ 7.44-7.28 (m, 5H), 6.81 (d, J=2.0 Hz, 1H), 6.46 (d, J=2.0 Hz, 1H), 4.75 (s, 2H).

### Intermediate Step 4 Toward Example 1

### 3-iodo-4-methoxy-aniline

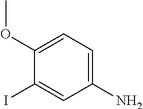

Dithionite (3.85 g, 22.15 mmol) as a slurry in water (20 mL) was added to mixture of 2-iodo-1-methoxy-4-nitro-benzene (1.03 g, 3.69 mmol) in MeOH (50 mL). The mixture was stirred for 5 m and then diluted with a saturated aq solution of NaHCO₃ (100 mL). The mixture was concentrated to half its volume under reduced pressure. The aq phase was extracted with EtOAc (3×50 mL), and the combined organic phases were washed with brine, dried over MgSO₄, filtered, and concentrated under reduced pressure. The product was purified by flash chromatography on silica gel, eluting with mixtures of hexanes and EtOAc to provide the title compound as a solid (0.652 g, 71%). ¹H NMR (300 MHz, CDCl₃) δ 7.16 (dd, J=0.8, 2.0 Hz, 1H), 6.68-6.64 (m, 2H), 3.79 (s, 3H), 3.42 (s, 2H).

### Intermediate Step 5 Toward Example 1

### 3-(3,5-dimethylisoxazol-4-yl)-4-methoxy-aniline

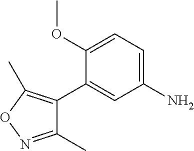 [SMILES: COc1ccc(N)cc1-c1c(C)noc1C]

Pd(OAc)₂ (1.42 g, 6.34 mmol) and PPh₃ (3.33 g, 12.7 mmol) were weighed into a 2-necked flask equipped with a reflux condenser, and the flask was flushed with N₂ gas. Degassed DME (60.0 mL) was added, and the mixture was stirred for 30 m at rt. 3,5-Dimethyl-4-(4,4,5,5-tetramethyl-1,3,2-dioxaborolan-2-yl)isoxazole (34.0 g, 152 mmol), 3-iodo-4-methoxy-aniline (15.8 g, 63.4 mmol), and Cs₂CO₃ (51.7 g, 159 mmol) were weighed into a second flask, and the flask was flushed with N₂ gas. Degassed DME (300 mL) and degassed water (30.0 mL) were added. The mixture was stirred for 20 m and then added to the first flask. The second flask was rinsed with degassed DME (15.0 mL), and the liquid was transferred to the first flask. The resulting mixture was heated to 90° C. for 16 h and then cooled to rt. The mixture was diluted with saturated aq NaHCO₃ (100 mL) and EtOAc (100 mL), and the aq phase was extracted with EtOAc (3×150 mL). The combined organic phases were dried over MgSO₄, filtered, and concentrated under reduced pressure. The product was purified by flash chromatography on silica gel, eluting with mixtures of EtOAc and hexanes to provide the title compound as a solid (12.9 g, 93%). ¹H NMR (500 MHz, CDCl₃) δ 6.80 (d, J=8.7 Hz, 1H), 6.69 (dd, J=8.6, 2.9 Hz, 1H), 6.48 (d, J=2.9 Hz, 1H), 3.70 (s, 3H), 3.49 (s, 2H), 2.29 (s, 3H), 2.16 (s, 3H); [M+H]+ 219.3.

### Example 1

### Ethyl 1-benzyl-7-(3,5-dimethylisoxazol-4-yl)-8-methoxy-2-oxo-oxazolo[5,4-c]quinoline-4-carboxylate

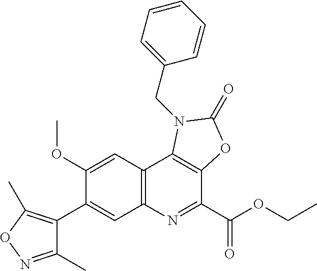 [SMILES: CCOC(=O)c1nc2cc(-c3c(C)noc3C)c(OC)cc2c2c1oc(=O)n2Cc1ccccc1]

Ethyl 2-oxoacetate (0.14 mL, 1.41 mmol, 50% in toluene) was added to a slurry of 3-(3,5-dimethylisoxazol-4-yl)-4-methoxy-aniline (140 mg, 0.641 mmol) and MgSO₄ (3.47 g, 28.9 mmol) in dry MeCN (10 mL). The mixture was stirred at rt for 2 h and then added via cannula to a second flask containing 3-benzyloxazol-2-one (169 mg, 0.962 mmol), Sc(OTf)₃ (316 mg, 0.641 mmol), and 4 Å molecular sieves (2.0 g). The residual MgSO₄ was diluted with dry MeCN (20 mL), and the liquid was transferred via cannula to the second flask. The mixture was stirred at 50° C. for 12 h. The mixture was filtered through Celite®, and the filtrate was concentrated under reduced pressure. The residue was diluted with water (100 mL) and EtOAc (100 mL). The aq phase was extracted with EtOAc (3×50 mL), and the combined organic phases were dried over MgSO₄, filtered, and concentrated under reduced pressure. The residue was dissolved in THF (20 mL), and pTSA (331 mg, 1.9 mmol) was added. The mixture was stirred open to the air for 12 h and then diluted with saturated aq NaHCO₃ (100 mL). The aq phase was extracted with EtOAc (3×50 mL). The combined organic phases were washed with brine, dried over MgSO₄, filtered, and concentrated under reduced pressure. The product was purified by flash chromatography on silica gel, eluting with mixtures of EtOAc and hexanes, followed by recrystallization from EtOAc and hexanes to provide the title compound as a solid (111.6 mg, 37%). ¹H NMR (300 MHz, CDCl₃) δ 8.04 (s, 1H), 7.45-7.31 (m, 5H), 7.00 (s, 1H), 5.57 (s, 2H), 4.62 (q, J=7.08 Hz, 2H), 3.56 (s, 3H), 2.29 (s, 3H), 2.12 (s, 3H), 1.52 (t, J=7.11 Hz, 3H); [M+H]⁺ 474.2.

### Example 2

### 1-Benzyl-7-(3,5-dimethylisoxazol-4-yl)-8-methoxy-oxazolo[5,4-c]quinolin-2-one

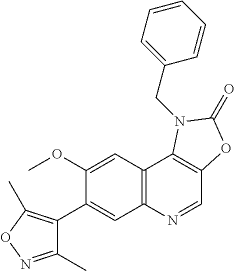 [SMILES: COc1cc2c(cc1-c1c(C)noc1C)ncc1oc(=O)n(Cc3ccccc3)c12]

3-(3,5-dimethylisoxazol-4-yl)-4-methoxy-aniline (see intermediate steps 4 and 5, Example 1, 423 mg, 1.938 mmol), Sc(OTf)₃ (286 mg, 0.581 mmol), and MgSO₄ (6.5 g, 54 mmol) were added to a flask, and the flask was flushed with N₂ gas. Paraformaldehyde (64 mg, 2.132 mmol) was added followed by dry MeCN (15 mL). The mixture was stirred for 30 m and then transferred to a second flask containing a slurry of 3-benzyloxazol-2-one (441 mg, 2.52 mmol), Sc(OTf)₃ (668 mg, 1.357 mmol), and 4 Å molecular sieves (5.54 g) in dry MeCN (15 mL). The residue in the first flask was diluted with additional MeCN (6 mL) and transferred to the second flask. The mixture was heated to 50° C. and stirred for 12 hours. The mixture was cooled to rt and filtered through a pad of Celite®, washing with EtOAc. The filtrate was diluted with saturated aq NaHCO₃ (100 mL) and EtOAc (100 mL), and the aq phase was extracted with EtOAc (3×50 mL). The combined organic fractions were dried over MgSO₄, filtered, and concentrated under reduced pressure. The product was purified by flash chromatography on silica gel, eluting with mixtures of EtOAc and hexanes to provide the saturated intermediate as a solid (0.6335 g, 81%). ¹HNMR (300 MHz, CDCl₃) δ 7.41-7.24 (m, 5H), 6.49 (s, 1H), 6.46 (s, 1H), 5.04 (dt, J=2.7, 9.2 Hz, 1H), 4.87 (d, J=16.2 Hz, 1H), 4.72 (d, J=9.2 Hz, 1H), 3.86 (d, J=16.2 Hz, 1H), 3.70 (br s, 1H), 3.64 (s, 3H), 3.54 (dd, J=2.9, 13.2 Hz, 1H), 3.15 (dd, J=2.9, 12.5 Hz, 1H), 2.29 (s, 3H), 2.14 (s, 3H); [M+H]⁺ 406.2. DDQ (422 mg, 1.86 mmol) and the solid from above (377 mg, 0.93 mmol) were weighed into a dry flask, and the flask was flushed with N₂ gas. Degassed MTBE (40 mL) was added, and the mixture was stirred for 12 h. The mixture was concentrated under reduced pressure, and the product was purified by flash chromatography on silica gel, eluting with mixtures of hexanes and EtOAc, followed by recrystallization from EtOAc and DCM to provide the title compound as a solid (142 mg, 38%). ¹HNMR (300 MHz, CDCl₃) δ 8.81 (s, 1H), 7.86 (s, 1H), 7.45-7.28 (m, 5H), 6.99 (s, 1H), 5.54 (s, 2H), 3.55 (s, 3H), 2.30 (s, 3H), 2.14 (s, 3H); [M+H]⁺ 402.1.

### Example 3

### 7-(3,5-Dimethylisoxazol-4-yl)-8-methoxy-1-[(1R)-1-(2-pyridyl)ethyl]oxazolo[5,4-c]quinolin-2-one

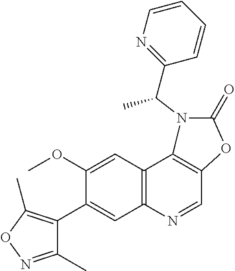 [SMILES: COc1cc2c(cc1-c1c(C)noc1C)ncc1oc(=O)n([C@H](C)c3ccccn3)c12]

A mixture of PPh₃ (125 mg, 0.476 mmol) in dry THF (5.00 mL) was cooled to 0° C., and DIAD (93 μL, 0.472 mmol) was added. A mixture of 7-(3,5-dimethylisoxazol-4-yl)-8-methoxy-1H-oxazolo[5,4-c]quinolin-2-one (see example 11, 57.0 mg, 0.183 mmol) in dry THF (5.00 mL) was added. The mixture was stirred at 0° C. for 10 m, and a solution of (1S)-1-(2-pyridyl)ethanol (65.4 mg, 0.531 mmol) in dry THF (5.00 mL) was added. The mixture was warmed to rt and stirred for 3 d. The mixture was diluted with EtOAc (100 mL) and a saturated aq NaHCO₃ (100 mL). The aq phase was extracted with EtOAc (3×75.0 mL), and the combined organic phases were washed with brine, dried over MgSO₄, filtered, and concentrated under reduced pressure. The product was purified by HPLC (high pH) to provide the title compound as a solid (13.1 mg, 17%). ¹H NMR (500 MHz, CDCl₃) δ 8.79 (s, 1H), 8.64 (d, J=4.3 Hz, 1H), 7.84 (s, 1H), 7.70 (td, J=7.8, 1.7 Hz, 1H), 7.45 (d, J=5.9 Hz, 1H), 7.29 (dd, J=7.5, 4.9 Hz, 1H), 7.11-6.86 (m, 1H), 6.22 (q, J=7.1 Hz, 1H), 3.64 (s, 3H), 2.30 (s, 3H), 2.16 (s, 3H), 2.15 (d, J=10.7 Hz, 3H); [M+H]⁺ 417.5.

### Intermediate Step 1 Toward Example 4

### 3-Phenyloxazol-2-one

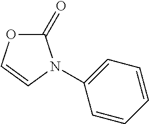 [SMILES: O=c1occn1-c1ccccc1]

This material can be synthesized according to the procedure outlined in Intermediate steps 1-2, Example 12A by replacing (1R)-1-phenylethanamine with aniline.

### Example 4

### 7-(3,5-Dimethylisoxazol-4-yl)-8-methoxy-1-phenyl-oxazolo[5,4-c]quinolin-2-one

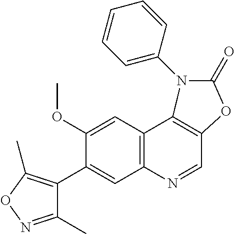 [SMILES: COc1cc2c(cc1-c1c(C)noc1C)ncc1oc(=O)n(-c3ccccc3)c12]

This material can be synthesized according to the procedure outlined in Example 2 for 1-benzyl-7-(3,5-dimethylisoxazol-4-yl)-8-methoxy-oxazolo[5,4-c]quinolin-2-one, replacing ethyl 3-benzyloxazol-2-one with 3-phenyloxazol-2-one.

### Intermediate Step 1 Toward Example 5

### 3-(2-Pyridyl)oxazol-2-one

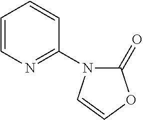 [SMILES: O=c1occn1-c1ccccn1]

This material can be synthesized according to the procedure outlined in intermediate steps 1-2, Example 12A by replacing (1R)-1-phenylethanamine with 2-aminopyridine.

### Example 5

### 7-(3,5-Dimethylisoxazol-4-yl)-8-methoxy-1-(2-pyridyl)oxazolo[5,4-c]quinolin-2-one

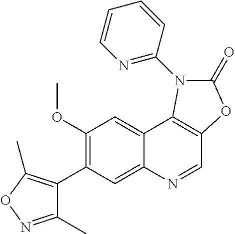 [SMILES: COc1cc2c(cc1-c1c(C)noc1C)ncc1oc(=O)n(-c3ccccn3)c12]

This material can be synthesized according to the procedure outlined in Example 2 for 1-benzyl-7-(3,5-dimethylisoxazol-4-yl)-8-methoxy-oxazolo[5,4-c]quinolin-2-one, replacing ethyl 3-benzyloxazol-2-one with 3-(2-pyridyl)oxazol-2-one.

### Intermediate Step 1 Toward Example 6

### 3-(Cyclohexylmethyl)oxazol-2-one

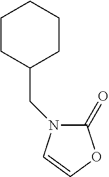 [SMILES: O=c1occn1CC1CCCCC1]

This material can be synthesized according to the procedure outlined in intermediate steps 1-2, Example 12A by replacing (1R)-1-phenylethanamine with cyclohexylmethanamine.

### Example 6

### 1-(Cyclohexylmethyl)-7-(3,5-dimethylisoxazol-4-yl)-8-methoxy-oxazolo[5,4-c]quinolin-2-one

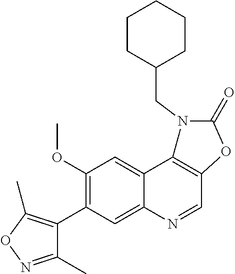 [SMILES: COc1cc2c(cc1-c1c(C)noc1C)ncc1oc(=O)n(CC3CCCCC3)c12]

This material can be synthesized according to the procedure outlined in Example 2 for 1-benzyl-7-(3,5-dimethylisoxazol-4-yl)-8-methoxy-oxazolo[5,4-c]quinolin-2-one, replacing ethyl 3-benzyloxazol-2-one with 3-(cyclohexylmethyl)oxazol-2-one.

### Intermediate Step 1 Toward Example 7

### 3-(Tetrahydropyran)-3-yloxazol-2-one

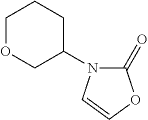 [SMILES: O=c1occn1C1CCCOC1]

This material can be synthesized according to the procedure outlined in intermediate steps 1-2, Example 12A by replacing (1R)-1-phenylethanamine with tetrahydropyran-3-amine.

### Example 7

### 7-(3,5-Dimethylisoxazol-4-yl)-8-methoxy-1-tetrahydropyran-3-yl-oxazolo[5,4c]quinolin-2-one

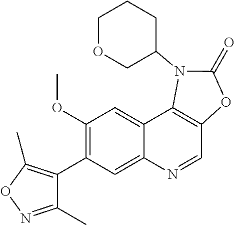 [SMILES: COc1cc2c(cc1-c1c(C)noc1C)ncc1oc(=O)n(C3CCCOC3)c12]

This material can be synthesized according to the procedure outlined in Example 2 for 1-benzyl-7-(3,5-dimethylisoxazol-4-yl)-8-methoxy-oxazolo[5,4-c]quinolin-2-one, replacing ethyl 3-benzyloxazol-2-one with 3-tetrahydropyran-3-yloxazol-2-one.

### Intermediate Step 1 Toward Example 8

### 3-(Tetrahydropyran-2-ylmethyl)oxazol-2-one

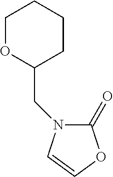 [SMILES: O=c1occn1CC1CCCCO1]

This material can be synthesized according to the procedure outlined in intermediate steps 1-2, Example 12A by replacing (1R)-1-phenylethanamine with tetrahydropyran-2-ylmethanamine.

### Example 8

### 7-(3,5-Dimethylisoxazol-4-yl)-8-methoxy-1-(tetrahydropyran-2-ylmethyl)oxazolo[5,4-c]quinolin-2-one

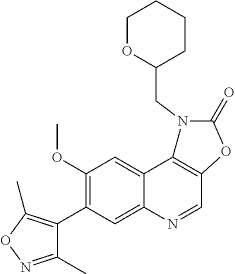 [SMILES: COc1cc2c(cc1-c1c(C)noc1C)ncc1oc(=O)n(CC3CCCCO3)c12]

This material can be synthesized according to the procedure outlined in Example 2 for 1-benzyl-7-(3,5-dimethylisoxazol-4-yl)-8-methoxy-oxazolo[5,4-c]quinolin-2-one, replacing ethyl 3-benzyloxazol-2-one with 3-(tetrahydropyran-2-ylmethyl)oxazol-2-one.

### Intermediate Step 1 Toward Example 9

### tert-Butyl 2-[(2-oxooxazol-3-yl)methyl]piperidine-1-carboxylate

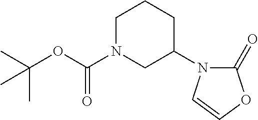 [SMILES: CC(C)(C)OC(=O)N1CCCC(n2ccoc2=O)C1]

This material can be synthesized according to the procedure outlined in Intermediate steps 1-2, 12A by replacing (1R)-1-phenylethanamine with tert-butyl 2-(aminomethyl)piperidine-1-carboxylate.

### Example 9

### 7-(3,5-Dimethylisoxazol-4-yl)-8-methoxy-1-(2-piperidylmethyl)oxazolo[5,4-c]quinolin-2-one

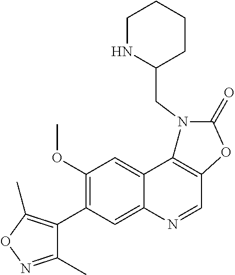 [SMILES: COc1cc2c(cc1-c1c(C)noc1C)ncc1oc(=O)n(CC3CCCCN3)c12]

This material can be synthesized according to the procedure outlined in Example 2 for 1-benzyl-7-(3,5-dimethylisoxazol-4-yl)-8-methoxy-oxazolo[5,4-c]quinolin-2-one, replacing ethyl 3-benzyloxazol-2-one with tert-butyl 2-[(2-oxooxazol-3-yl)methyl]piperidine-1-carboxylate. The resulting intermediate can be mixed with a suitable acid such as TFA in a suitable solvent such as DCM. The resulting mixture can then be isolated and purified by methods standard in the art to provide the title compound.

### Intermediate Step 1 Toward Example 10

### 3-Allyloxazol-2-one

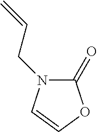

This material can be synthesized according to the procedure outlined in intermediate steps 1-2, Example 12A by replacing (1R)-1-phenylethanamine with allylamine.

### Example 10

### 1-Allyl-7-(3,5-dimethylisoxazol-4-yl)-8-methoxy-oxazolo[5,4-c]quinolin-2-one

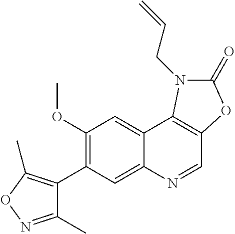 [SMILES: C=CCn1c(=O)oc2cnc3cc(-c4c(C)noc4C)c(OC)cc3c21]

This material can be synthesized according to the procedure outlined in Example 2 for 1-benzyl-7-(3,5-dimethylisoxazol-4-yl)-8-methoxy-oxazolo[5,4-c]quinolin-2-one, replacing ethyl 3-benzyloxazol-2-one with 3-allyloxazol-2-one.

### Intermediate Step 1 Towards Example 11

### 3-tert-Butyloxazolidine-2,4-dione

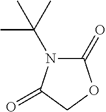

A mixture of tert-butylamine (6.98 g, 10.03 mL, 95.5 mmol), ethyl glycolate (9.94 g, 9.04 mL, 95.5 mmol), and NaOMe (4.13 mL, 19.1 mmol) was stirred at 120° C. for 2 h. The mixture was cooled to rt and concentrated under reduced pressure. The residue was dissolved in DMF (80.0 mL), and CDI (17.0 g, 105 mmol) was added. The mixture was stirred at rt for 2 h. The mixture diluted with brine (500 mL). The aq phase was extracted into EtOAc (3×100 mL). The combined organic phases were washed with brine (3×100 mL), dried over MgSO₄, filtered, and concentrated under reduced pressure to provide the title compound as a solid (12.4 g, 83%). ¹H NMR (500 MHz, CDCl₃) δ 4.49 (s, 2H), 1.60 (s, 9H).

### Intermediate Step 2 Towards Example 11

### 3-tert-Butyloxazol-2-one

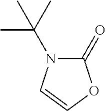

The solid from above (12.4 g, 79.0 mmol) was dissolved in MeOH (200 mL), and the mixture was cooled to 0° C. NaBH₄ (8.97 g, 237 mmol) was slowly added in small portions over 2 h. The mixture was slowly warmed to rt and stirred for 20 m. The mixture was cooled to 0° C., and acetone (50.0 mL) was slowly added. The mixture was slowly warmed to rt and stirred for 10 m. The mixture was concentrated under reduced pressure, and the residue was diluted with EtOAc (100 mL) and saturated aq NaHCO₃ (100 mL). The aq phase was extracted with EtOAc (3×100 mL), and the combined organic phases were dried over MgSO₄, filtered, and concentrated under reduced pressure to provide 3-tert-butyl-4-hydroxy-oxazolidin-2-one as an oil (7.26 g, 58%). ¹H NMR (500 MHz, CDCl₃) δ 5.37 (d, J=5.0 Hz, 1H), 4.22 (dd, J=5.8, 9.9 Hz, 1H), 4.14 (s, 1H), 4.10 (dd, J=9.9, 1.0 Hz, 1H), 1.48 (s, 9H). The oil from above (188 mg, 1.18 mmol) was diluted with DCE (10.0 mL), and TEA (3.10 mL, 24.0 mmol) and DMAP (14.4 mg, 0.118 mmol) were added. The mixture was cooled to 0° C., and MsCl (149 mg, 1.30 mmol, 0.101 mL) was slowly added. The mixture was heated to 65° C. and stirred for 4 h. The mixture was cooled to rt and diluted with saturated aq NH₄Cl (100 mL). The aq phase was extracted with EtOAc (3×75.0 mL), and the combined organic phases were washed with brine (100 mL), dried over MgSO₄, filtered, and concentrated under reduced pressure. The product was purified by flash chromatography on silica gel, eluting with mixtures of EtOAc and hexanes to provide the title compound as a solid (1.55 g, 26%). ¹H NMR (500 MHz, CDCl₃) δ 6.73 (d, J=2.1 Hz, 1H), 6.60 (d, J=2.1 Hz, 1H), 1.51 (s, 9H).

### Example 11

### 7-(3,5-Dimethylisoxazol-4-yl)-8-methoxy-1H-oxazolo[5,4-c]quinolin-2-one

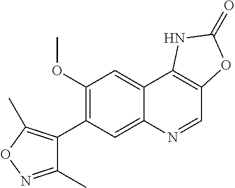 [SMILES: COc1cc2c(cc1-c1c(C)noc1C)ncc1oc(=O)[nH]c12]

A mixture of 3-(3,5-dimethylisoxazol-4-yl)-4-methoxy-aniline (see intermediate steps 4 and 5, Example 1, 2.13 g, 9.78 mmol), MgSO₄ (35.3 g, 293 mmol), Sc(OTf)₃ (874 mg, 1.76 mmol), and paraformaldehyde (320 mg, 10.8 mmol) in dry MeCN (40.0 mL) was stirred at rt for 2 h and then added to another mixture of 3-tert-butyloxazol-2-one (1.38 mg, 9.78 mmol), Sc(OTf)₃ (1.25 mg, 2.54 mmol), and 4 Å molecular sieves (2.00 g). The residual MgSO₄ in the first flask was rinsed with dry MeCN (80.0 mL) and transferred to the second. The resulting mixture was heated to 50° C. and stirred for 16 h. The mixture was filtered through a pad of Celite®, washing with EtOAc and DCM. The mixture was concentrated under reduced pressure to provide the saturated intermediate as a solid. [M+H]⁺ 372.4. DDQ (4.66 g, 20.5 mmol) was added to the solid from above (3.63 g, 9.77 mmol), and the flask was dried under high vacuum and then flushed with N₂ gas. Degassed MTBE (220 mL) was added, and the mixture was heated to 60° C. and stirred for 4 h. The mixture was diluted with saturated aq NaHCO₃ (100 mL) and EtOAc (100 mL). The aq phase was extracted with EtOAc (3×75.0 mL), and the combined organic phases were washed with brine, dried over MgSO₄, filtered, and concentrated under reduced pressure. The product was purified by flash chromatography on silica gel, eluting with mixtures of EtOAc and hexanes to provide the unsaturated intermediate as a solid (480 mg, 13%). ¹H NMR (500 MHz, CDCl₃) δ 8.70 (s, 1H), 7.90 (s, 1H), 7.61 (s, 1H), 3.94 (s, 3H), 2.33 (s, 3H), 2.19 (s, 3H), 1.99 (s, 9H); [M+H]⁺ 368.4. A solution of the purified material from above (300 mg, 0.82 mmol) in TFA (25.0 mL) was stirred at rt for 12 h. The mixture was concentrated under reduced pressure, and the residue was diluted with saturated aq NaHCO₃ (75.0 mL) and DCM (75.0 mL). The aq phase was extracted with DCM (3×75.0 mL), and the combined organic phases were washed with brine (100 mL), dried over MgSO₄, filtered, and concentrated under reduced pressure. The product was purified by flash chromatography on silica gel, eluting with mixtures of EtOAc and hexanes to provide the title compound as a solid (180 mg, 71%). ¹H NMR (500 MHz, MeOD) δ 8.67 (s, 1H), 7.84 (s, 1H), 7.58 (s, 1H), 4.02 (s, 3H), 2.37 (s, 3H), 2.20 (s, 3H); [M+H]⁺ 312.3.

### Example 12

### 7-(3,5-Dimethylisoxazol-4-yl)-8-methoxy-1-(1-phenylethyl)oxazolo[5,4-c]quinolin-2-one

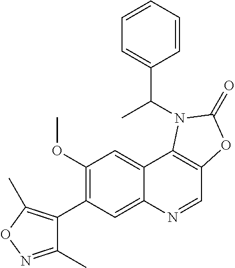 [SMILES: COc1cc2c(cc1-c1c(C)noc1C)ncc1oc(=O)n(C(C)c3ccccc3)c12]

This material can be synthesized by stirring 7-(3,5-dimethylisoxazol-4-yl)-8-methoxy-1H-oxazolo[5,4-c]quinolin-2-one with a suitable base such as cesium carbonate and 1-(bromoethyl)benzene in a suitable solvent such as DMF for approx. 12 hours.

### Intermediate Step 1 Towards Example 12a

### 3-[(1R)-1-Phenylethyl]oxazolidine-2,4-dione

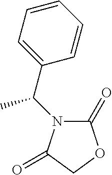

A mixture of (1R)-1-phenylethanamine (5.00 g, 41.3 mmol), ethyl glycolate (4.30 g, 41.3 mmol, 3.91 mL), and NaOMe (4.6M in MeOH, 4.13 mmol, 0.890 mL) was heated to 120° C. and stirred for 1.5 h. The mixture was cooled to rt and concentrated under reduced pressure. The residue was dissolved in dry DMF (150 mL), CDI (4.82 g, 29.71 mmol) was added, and the mixture was stirred for 1 h. The mixture was concentrated under reduced pressure and then diluted with DCM (60.0 mL) and water (100 mL). The aqueous phase was extracted with DCM (3×60.0 mL), and the combined organic phases were dried over Na₂SO₄, filtered, and concentrated under reduced pressure to provide 3-[(1R)-1-phenylethyl]oxazolidine-2,4-dione as an oil (11.0 g, 100%). ¹H NMR (300 MHz, CDCl₃) δ 7.49 (dd, J=7.7, 1.6 Hz, 2H), 7.40-7.31 (m, 3H), 5.34 (q, J=7.6 Hz, 1H), 4.61 (s, 2H), 1.88 (d, J=7.4 Hz, 3H).

### Intermediate Step 2 Towards Example 12A

### 3-[(1R)-1-Phenylethyl]oxazol-2-one

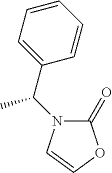

A mixture of 3-[(1R)-1-phenylethyl]oxazolidine-2,4-dione (11.0 g, 53.6 mmol) in MeOH (400 mL) was cooled to 0° C., and NaBH₄ (134 mmol, 5.1 g) was slowly added over 1 h. Acetone (50 mL) was slowly added, and the mixture was stirred for 15 m. The mixture was concentrated under reduced pressure, and the residue was diluted with DCM (50.0 mL) and water (200 mL). The aq phase was extracted with DCM (3×50.0 mL), and the combined organic phases were dried over Na₂SO₄, filtered, and concentrated under reduced pressure. The residue was dissolved in DCM (200 mL), and the mixture was cooled at 0° C. TEA (80 mmol, 11.2 mL) and MsCl (8.30 mL, 107 mmol) were added, and the mixture was stirred at rt for 30 m. The mixture was diluted with saturated aq NH₄Cl (250 mL), and the aq phase was extracted with DCM (3×150 mL). The combined organic phases were dried over Na₂SO₄, filtered, and concentrated under reduced pressure. The product was purified by flash chromatography on silica gel, eluting with mixtures of EtOAc and hexanes to provide the title compound as an oil (5.00 g, 49%). ¹H NMR (300 MHz, CDCl₃) δ 7.42-7.28 (m, 5H), 6.76 (d, J=2.1 Hz, 1H), 6.44 (d, J=2.1 Hz, 1H), 5.34 (q, J=7.1 Hz, 1H), 1.70 (d, J=7.1 Hz, 3H).

### Example 12A

### 7-(3,5-Dimethylisoxazol-4-yl)-8-methoxy-1-[(1R)-1-phenylethyl]oxazolo[5,4-c]quinolin-2-one

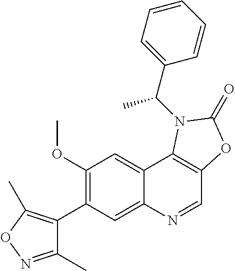 [SMILES: COc1cc2c(cc1-c1c(C)noc1C)ncc1oc(=O)n([C@H](C)c3ccccc3)c12]

Paraformaldehyde (434 mg, 14.5 mmol) was added to a mixture of 3-(3,5-dimethylisoxazol-4-yl)-4-methoxy-aniline (see intermediate steps 4 and 5, Example 1, 2.89 g, 13.2 mmol), MgSO₄ (63.3 g, 526 mmol), and Sc(OTf)₃ (1.94 g, 3.94 mmol) in dry MeCN (60.0 mL). The mixture was stirred at rt for 2 h and then added to a mixture of 3-[(1R)-1-phenylethyl]oxazol-2-one (2.99 g, 15.8 mmol), Sc(OTf)₃ (4.53 g, 9.20 mmol), and 4 Å molecular sieves (10.0 g). The residual MgSO₄ in the first flask was rinsed with dry MeCN (85.0 mL), and the liquid was transferred to the second. The resulting mixture was heated to 50° C. and stirred for 16 h. The mixture was filtered through a pad of Celite®, washing with DCM. The mixture was concentrated under reduced pressure, and the residue was diluted with saturated aq NaHCO₃ (100 mL) and DCM (100 mL). The aq phase was extracted with DCM (3×100 mL), and the combined organic phases were dried over MgSO₄, filtered, and concentrated under reduced pressure to provide 7-(3,5-dimethylisoxazol-4-yl)-8-methoxy-1-[(1R)-1-phenylethyl]-3a,4,5,9b-tetrahydrooxazolo[5,4-c]quinolin-2-one as a solid. [M+H]⁺ 420.4. DDQ (6.71 g, 29.6 mmol) was added to the solid from above (5.51 g, 13.2 mmol). The flask was dried under high vacuum and flushed with N₂ gas. Degassed MTBE (210 mL) was added, and the mixture was heated to 60° C. and stirred for 6 h. The mixture was cooled to rt and filtered through a pad of Celite®, washing with EtOAc. The filtrate was concentrated under reduced pressure. The residue was diluted with EtOAc (150 mL) and saturated aq NaHCO₃ (150 mL). The aq phase was extracted with EtOAc (3×100 mL), and the combined organic phases were washed with brine, dried over MgSO₄, filtered, and concentrated under reduced pressure. The product was purified by flash chromatography on silica gel, eluting with mixtures of EtOAc and hexanes, followed by recrystallization from DCM and MTBE to provide the title compound as a solid (0.802 g, 15%). ¹H NMR (500 MHz, MeOD) δ 9.25 (s, 1H), 8.01 (s, 1H), 7.60 (d, J=8.1 Hz, 2H), 7.47 (t, J=7.6 Hz, 2H), 7.41 (t, J=7.3 Hz, 1H), 6.97 (s, 1H), 6.36 (q, J=7.0 Hz, 1H), 3.52 (s, 3H), 2.31 (s, 3H), 2.15 (d, J=7.2 Hz, 3H), 2.12 (s, 3H); [M+H]⁺ 416.3.

### Example 13

### 7-(3,5-Dimethylisoxazol-4-yl)-8-methoxy-1-(3-pyridylmethyl)oxazolo[5,4-c]quinolin-2-one

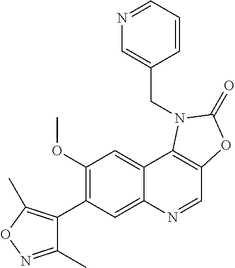 [SMILES: COc1cc2c(cc1-c1c(C)noc1C)ncc1oc(=O)n(Cc3cccnc3)c12]

This material can be synthesized according to the procedure outlined in Example 2 for 7-(3,5-dimethylisoxazol-4-yl)-8-methoxy-1-[(1R)-1-(2-pyridyl)ethyl]oxazolo[5,4-c]quinolin-2-one, replacing (1S)-1-(2-pyridyl)ethanol with 3-pyridylmethanol.

### Example 14

### 7-(3,5-Dimethylisoxazol-4-yl)-8-methoxy-1-[[5-(trifluoromethyl)-2-furyl]methyl]oxazolo[5,4-c]quinolin-2-one

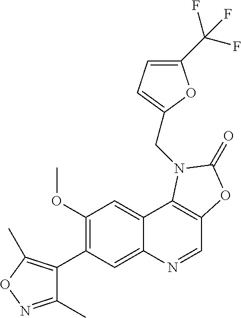 [SMILES: COc1cc2c(cc1-c1c(C)noc1C)ncc1oc(=O)n(Cc3ccc(C(F)(F)F)o3)c12]

This material can be synthesized according to the procedure outlined in Example 18 for 1-[(2-chlorophenyl)methyl]-7-(3,5-dimethylisoxazol-4-yl)-8-methoxy-oxazolo[5,4-c]quinolin-2-one, replacing 2-chlorobenzyl bromide with 2-(bromomethyl)-5-(trifluoromethyl)furan.

### Example 15

### 7-(3,5-Dimethylisoxazol-4-yl)-8-methoxy-1-(4-pyridylmethyl)oxazolo[5,4-c]quinolin-2-one

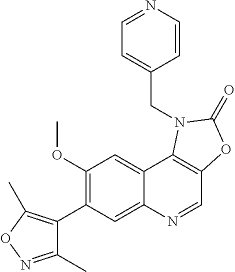 [SMILES: COc1cc2c(cc1-c1c(C)noc1C)ncc1oc(=O)n(Cc3ccncc3)c12]

This material can be synthesized according to the procedure outlined in Example 3 for 7-(3,5-dimethylisoxazol-4-yl)-8-methoxy-1-[(1R)-1-(2-pyridyl)ethyl]oxazolo[5,4-c]quinolin-2-one, replacing (1S)-1-(2-pyridyl)ethanol with 4-pyridylmethanol.

### Example 16

### 7-(3,5-Dimethylisoxazol-4-yl)-8-methoxy-1-(2-pyridylmethyl)oxazolo[5,4-c]quinolin-2-one

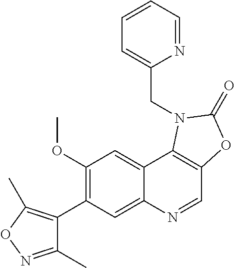 [SMILES: COc1cc2c(cc1-c1c(C)noc1C)ncc1oc(=O)n(Cc3ccccn3)c12]

This material can be synthesized according to the procedure outlined in Example 3 for 7-(3,5-dimethylisoxazol-4-yl)-8-methoxy-1-[(1R)-1-(2-pyridyl)ethyl]oxazolo[5,4-c]quinolin-2-one, replacing (1S)-1-(2-pyridyl)ethanol with 2-pyridylmethanol.

### Example 17

### 1-[(3-Chlorophenyl)methyl]-7-(3,5-dimethylisoxazol-4-yl)-8-methoxyoxazolo[5,4-c]quinolin-2-one

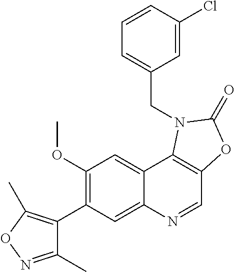 [SMILES: COc1cc2c(cc1-c1c(C)noc1C)ncc1oc(=O)n(Cc3cccc(Cl)c3)c12]

This material can be synthesized according to the procedure outlined in Example 2 for 1-[(2-chlorophenyl)methyl]-7-(3,5-dimethylisoxazol-4-yl)-8-methoxy-oxazolo[5,4-c]quinolin-2-one, replacing 2-chlorobenzyl bromide with 3-chlorobenzyl bromide.

### Example 18

### 1-[(2-Chlorophenyl)methyl]-7-(3,5-dimethylisoxazol-4-yl)-8-methoxy-oxazolo[5,4-c]quinolin-2-one

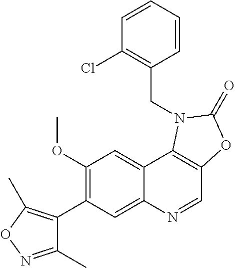 [SMILES: COc1cc2c(cc1-c1c(C)noc1C)ncc1oc(=O)n(Cc3ccccc3Cl)c12]

A mixture of 7-(3,5-dimethylisoxazol-4-yl)-8-methoxy-1H-oxazolo[5,4-c]quinolin-2-one (see example 11, 31.1 mg, 0.100 mmol), Cs₂CO₃ (65.1 mg, 0.199 mmol), TBAI (36.9 mg, 0.0999 mmol), and 2-chlorobenzyl bromide (25.9 μL, 199.8 mmol) in dry DMF (5.00 mL) was stirred at rt for 16 h. The mixture was concentrated under reduced pressure, and the residue was diluted with EtOAc (25.0 mL) and saturated aq NaHCO₃ (25.0 mL). The aq phase was extracted with EtOAc (3×25.0 mL), and the combined organic phases were washed with brine (30.0 mL), dried over Na₂SO₄, filtered, and concentrated under reduced pressure. The residue was purified by flash chromatography on silica gel, eluting with mixtures of EtOAc and hexanes to provide the title compound as a solid (7 mg, 16%). ¹H NMR (500 MHz, CDCl₃) δ 8.83 (s, 1H), 7.87 (s, 1H), 7.54 (dd, J=8.0, 1.1 Hz, 1H), 7.33 (td, J=8.0, 1.5 Hz, 1H), 7.24 (td, J=7.7, 1.2 Hz, 1H), 7.12-7.03 (m, 1H), 6.76 (s, 1H), 5.63 (s, 2H), 3.55 (s, 3H), 2.30 (s, 3H), 2.14 (s, 3H); [M+H]⁺ 436.4.

### Example 19

### 7-(3,5-Dimethylisoxazol-4-yl)-8-methoxy-1-[(3-methoxyphenyl)methyl]oxazolo[5,4-c]quinolin-2-one

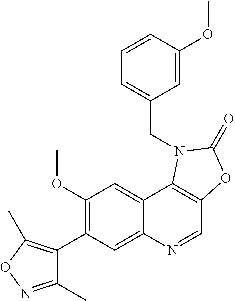 [SMILES: COc1cccc(Cn2c(=O)oc3cnc4cc(-c5c(C)noc5C)c(OC)cc4c32)c1]

This material can be synthesized according to the procedure outlined in Example 18 for 1-[(2-chlorophenyl)methyl]-7-(3,5-dimethylisoxazol-4-yl)-8-methoxy-oxazolo[5,4-c]quinolin-2-one, replacing 2-chlorobenzyl bromide with 3-methoxybenzyl bromide.

### Example 20

### 7-(3,5-Dimethylisoxazol-4-yl)-8-methoxy-1-[(2-methoxyphenyl)methyl]oxazolo[5,4-c]quinolin-2-one

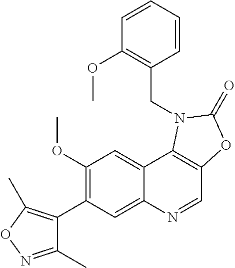 [SMILES: COc1ccccc1Cn1c(=O)oc2cnc3cc(-c4c(C)noc4C)c(OC)cc3c21]

A mixture of 7-(3,5-dimethylisoxazol-4-yl)-8-methoxy-1H-oxazolo[5,4-c]quinolin-2-one (22.9 mg, 0.0700 mmol, see example 11), Cs₂CO₃ (47.9 mg, 0.150 mmol), TBAI (27.2 mg, 0.0700 mmol), and 1-(chloromethyl)-2-methoxy-benzene (23.0 mg, 0.150 mmol) in dry DMF (5.00 mL) was stirred at rt for 12 h. The mixture was concentrated under reduced pressure, and the residue was diluted with EtOAc (75.0 mL) and saturated aq NaHCO₃ (75.0 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) to provide the title compound as a solid (1.94 mg, 6.4%). ¹H NMR (500 MHz, CDCl₃) δ 8.80 (s, 1H), 7.85 (s, 1H), 7.39-7.28 (m, 1H), 7.04 (d, J=7.0 Hz, 1H), 6.98 (d, J=7.8 Hz, 1H), 6.97 (s, 1H), 6.91 (t, J=7.5 Hz, 1H), 5.53 (s, 2H), 3.92 (s, 3H), 3.52 (s, 3H), 2.30 (s, 3H), 2.14 (s, 3H); [M+H]⁺ 432.3.

### Example 21

### 1-Benzyl-7-(3,5-dimethylisoxazol-4-yl)-8-methoxy-2-oxo-oxazolo[5,4-c]quinoline-4-carboxylic acid

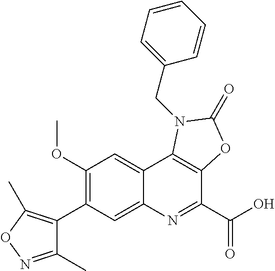 [SMILES: COc1cc2c(cc1-c1c(C)noc1C)nc(C(=O)O)c1oc(=O)n(Cc3ccccc3)c12]

The material from Example 1 can be heated with a suitable acid such as 6N HCl for approx 12 h. The resulting mixture can be concentrated under reduced pressure. The product can be isolated and purified by methods standard in the art to provide the title compound.

### Example 22

### 1-Benzyl-7-(3,5-dimethylisoxazol-4-yl)-8-methoxy-N-methyl-2-oxo-oxazolo[5,4-c]quinoline-4-carboxamide

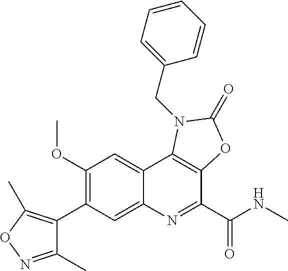 [SMILES: CNC(=O)c1nc2cc(-c3c(C)noc3C)c(OC)cc2c2c1oc(=O)n2Cc1ccccc1]

The material from Example 21 can be mixed with methylamine hydrochloride and a suitable dehydrating agent such as HATU and a suitable base such as TEA. The resulting mixture can then be isolated and purified by methods standard in the art to provide the title compound.

### Example 23

### 1-Benzyl-7-(3,5-dimethylisoxazol-4-yl)-8-methoxy-N,N-dimethyl-2-oxo-oxazolo[5,4-c]quinoline-4-carboxamide

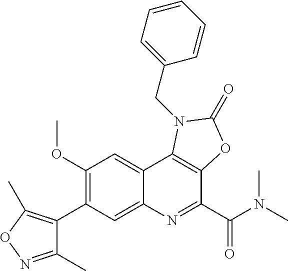 [SMILES: COc1cc2c(cc1-c1c(C)noc1C)nc(C(=O)N(C)C)c1oc(=O)n(Cc3ccccc3)c12]

The material from Example 21 can be mixed with dimethylamine hydrochloride and a suitable dehydrating agent such as HATU and a suitable base such as TEA. The resulting mixture can then be isolated and purified by methods standard in the art to provide the title compound.

### Example 24

### 1-Benzyl-7-(3,5-dimethylisoxazol-4-yl)-8-methoxy-4-(morpholine-4-carbonyl)oxazolo[5,4-c]quinolin-2-one

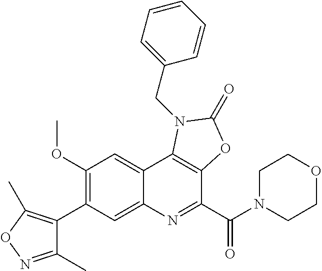 [SMILES: COc1cc2c(cc1-c1c(C)noc1C)nc(C(=O)N1CCOCC1)c1oc(=O)n(Cc3ccccc3)c12]

The material from Example 21 can be mixed with morpholine and a suitable dehydrating agent such as HATU and a suitable base such as TEA. The resulting mixture can then be isolated and purified by methods standard in the art to provide the title compound.

### Example 25

### 1-Benzyl-4-cyclohexyl-7-(3,5-dimethylisoxazol-4-yl)-8-methoxy-oxazolo[5,4-c]quinolin-2-one

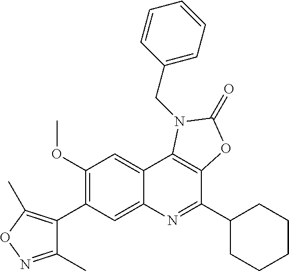 [SMILES: COc1cc2c(cc1-c1c(C)noc1C)nc(C1CCCCC1)c1oc(=O)n(Cc3ccccc3)c12]

This material can be synthesized according to the procedure outlined in Example 2, replacing paraformaldehye with cyclohexanecarbaldehyde.

### Example 26

### 1-Benzyl-7-(3,5-dimethylisoxazol-4-yl)-8-methoxy-4-(4-piperidyl)oxazolo[5,4-c]quinolin-2-one

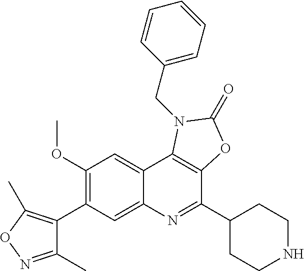 [SMILES: COc1cc2c(cc1-c1c(C)noc1C)nc(C1CCNCC1)c1oc(=O)n(Cc3ccccc3)c12]

This material can be synthesized according to the procedure outlined in Example 2, replacing paraformaldehyde with tert-butyl 4-formylpiperidine-1-carboxylate. The resulting intermediate can then be isolated and purified by methods standard in the art. The intermediate can be mixed with a suitable acid such as TFA in a suitable solvent such as DCM. The resulting mixture can then be isolated and purified by methods standard in the art to provide the title compound.

### Example 27

### 1-Benzyl-7-(3,5-dimethylisoxazol-4-yl)-8-methoxy-4-(methoxymethyl)oxazolo[5,4-c]quinolin-2-one

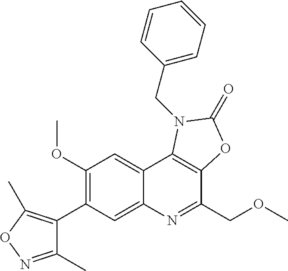 [SMILES: COCc1nc2cc(-c3c(C)noc3C)c(OC)cc2c2c1oc(=O)n2Cc1ccccc1]

The material from Example 33 can be mixed with MsCl and a suitable base such as TEA in a suitable solvent such as DCM, and the mixture can be concentrated under reduced pressure. The residue can be refluxed in MeOH with a suitable base such as DMAP. The resulting mixture can then be isolated and purified by methods standard in the art to provide the title compound.

Example 28: 1-Benzyl-7-(3,5-dimethylisoxazol-4-yl)-8-methoxy-4-(phenoxymethyl)oxazolo[5,4-c]quinolin-2-one

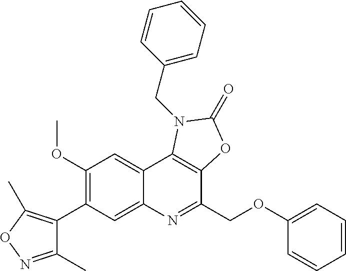 [SMILES: COc1cc2c(cc1-c1c(C)noc1C)nc(COc1ccccc1)c1oc(=O)n(Cc3ccccc3)c12]

The material from Example 33 can be mixed with DIAD, PPh₃, and phenol in a suitable solvent such as THF. The resulting mixture can then be isolated and purified by methods standard in the art to provide the title compound.

### Example 29

### 1-Benzyl-7-(3,5-dimethylisoxazol-4-yl)-8-methoxy-4-phenyl-oxazolo[5,4-c]quinolin-2-one

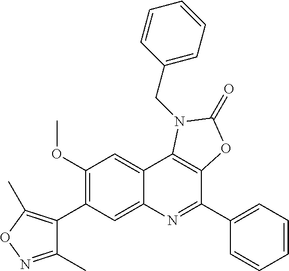 [SMILES: COc1cc2c(cc1-c1c(C)noc1C)nc(-c1ccccc1)c1oc(=O)n(Cc3ccccc3)c12]

This material can be synthesized according to the procedure outlined in Example 2, replacing paraformaldehyde with benzaldehyde.

### Example 30

### 1-Benzyl-7-(3,5-dimethylisoxazol-4-yl)-4-(1H-imidazol-4-yl)-8-methoxy-oxazolo[5,4-c]quinolin-2-one

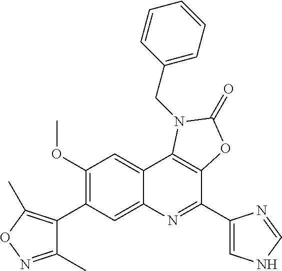 [SMILES: COc1cc2c(cc1-c1c(C)noc1C)nc(-c1c[nH]cn1)c1oc(=O)n(Cc3ccccc3)c12]

This material can be synthesized according to the procedure outlined in Example 2, replacing paraformaldehyde with tert-butyl 4-formylimidazole-1-carboxylate. The resulting intermediate can then be isolated and purified by methods standard in the art. The intermediate can be mixed with a suitable acid such as TFA in a suitable solvent such as DCM. The resulting mixture can then be isolated and purified by methods standard in the art to provide the title compound.

### Example 31

### 1-Benzyl-7-(3,5-dimethylisoxazol-4-yl)-4-isobutyl-8-methoxy-oxazolo[5,4-c]quinolin-2-one

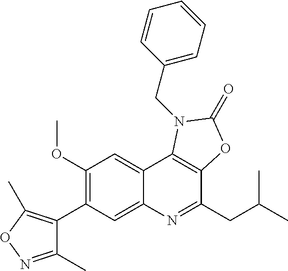 [SMILES: COc1cc2c(cc1-c1c(C)noc1C)nc(CC(C)C)c1oc(=O)n(Cc3ccccc3)c12]

This material can be synthesized according to the procedure outlined in Example 2, replacing paraformaldehyde with 3-methylbutanal.

### Example 32

### 1,4-Dibenzyl-7-(3,5-dimethylisoxazol-4-yl)-8-methoxy-oxazolo[5,4-c]quinolin-2-one

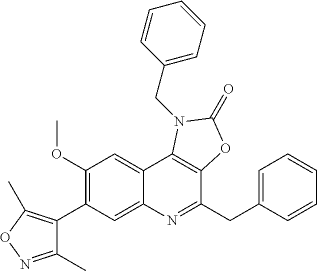 [SMILES: COc1cc2c(cc1-c1c(C)noc1C)nc(Cc1ccccc1)c1oc(=O)n(Cc3ccccc3)c12]

This material can be synthesized according to the procedure outlined in Example 2, replacing paraformaldehyde with 2-phenylacetaldehyde.

### Intermediate Step 1 Towards Example 33

### 1-Benzyl-7-(3,5-dimethylisoxazol-4-yl)-8-methoxy-2-oxo-oxazolo[5,4-c]quinoline-4-carbaldehyde

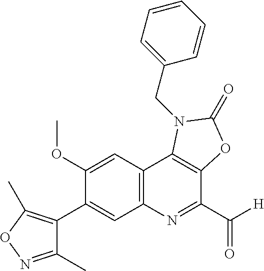 [SMILES: COc1cc2c(cc1-c1c(C)noc1C)nc(C=O)c1oc(=O)n(Cc3ccccc3)c12]

Ethyl 1-benzyl-7-(3,5-dimethylisoxazol-4-yl)-8-methoxy-2-oxo-oxazolo[5,4-c]quinoline-4-carboxylate (see Example 1, 85.1 mg, 0.180 mmol) was dissolved in dry THF (20.0 mL), and the mixture was cooled to −78° C. DIBAL (0.540 mL, 0.539 mmol, 1.00 M in toluene) was added dropwise, and the mixture was stirred for 1 h at −78° C. The reaction was diluted with EtOH (6.00 mL) at −78° C. and stirred for 5 m. The mixture was diluted with saturated aqueous Na/K tartrate (100 mL) and stirred for 2 h at rt. The organic phase was extracted with DCM (3×20.0 mL), and the combined organic phases were dried over Na₂SO₄, filtered, and concentrated under reduced pressure. The product was purified by flash chromatography on silica gel, eluting with mixtures of EtOAc and hexanes to provide the title compound as a solid (50.0 mg, 65%). [M+H]⁺ 430.2.

### Example 33

### 1-Benzyl-7-(3,5-dimethylisoxazol-4-yl)-4-(hydroxymethyl)-8-methoxy-oxazolo[5,4-c]quinolin-2-one

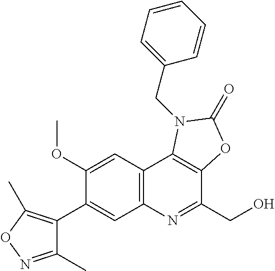 [SMILES: COc1cc2c(cc1-c1c(C)noc1C)nc(CO)c1oc(=O)n(Cc3ccccc3)c12]

NaBH₄ (1.32 mg, 0.0350 mmol) was added to a solution of 1-benzyl-7-(3,5-dimethylisoxazol-4-yl)-8-methoxy-2-oxo-oxazolo[5,4-c]quinoline-4-carbaldehyde (10.0 mg, 0.0230 mmol) in MeOH (5.00 mL), and the mixture was stirred for 1 h. The mixture was filtered through a pad of Celite®, washing with MeOH. The filtrate was concentrated under reduced pressure. The product was purified by preparative TLC to provide the title compound as a solid (5.00 mg, 49%). ¹H NMR (300 MHz, CDCl₃) δ 7.85 (s, 1H), 7.50-7.30 (m, 5H), 7.00 (s, 1H), 5.54 (s, 2H), 5.08 (s, 2H), 3.96 (s, 1H), 3.56 (s, 3H), 2.31 (s, 3H), 2.14 (s, 3H); [M+H]⁺ 432.2.

### Example 34

### Ethyl 7-(3,5-Dimethylisoxazol-4-yl)-8-methoxy-2-oxo-1-[(1R)-1-phenylethyl]oxazolo[5,4-c]quinoline-4-carboxylate

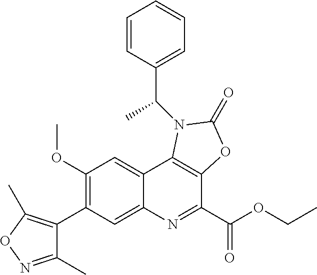 [SMILES: CCOC(=O)c1nc2cc(-c3c(C)noc3C)c(OC)cc2c2c1oc(=O)n2[C@H](C)c1ccccc1]

A mixture of 3-(3,5-dimethylisoxazol-4-yl)-4-methoxy-aniline (see Intermediate steps 4 and 5, Example 1, 515 mg, 2.72 mmol), Sc(OTf)₃ (280 mg, 0.570 mmol), MgSO₄ (8.93 g, 74.2 mmol), and ethyl glyoxylate (0.540 mL, 5.44 mmol, 50% in toluene) in dry MeCN (20.0 mL) was stirred at rt for 1 h and then added to a mixture of 3-[(1R)-1-phenylethyl]oxazol-2-one (see Intermediate steps 1 and 2, Example 12, 515 g, 2.72 mmol), Sc(OTf)₃ (280 mg, 0.570 mmol), and 4 Å molecular sieves (1.00 g). The residual MgSO₄ was rinsed with MeCN (20.0 mL), and the liquid was transferred to the reaction mixture. The mixture was heated to 50° C. and stirred for 12 h. The mixture was filtered through a pad of Celite®, washing with EtOAc, and the filtrate was concentrated under reduced pressure. The residue was diluted with EtOAc (100 mL) and saturated aq NaHCO₃ (100 mL). The aq phase was extracted with EtOAc (3×75.0 mL), and the combined organic phases were washed with brine (100 mL), dried over MgSO₄, filtered, and concentrated under reduced pressure to provide the unsaturated intermediate as a solid. [M+H]⁺ 492.5. DDQ (1.26 g, 5.55 mmol) was added to the solid from above (1.22 g, 2.64 mmol), and the flask was dried under high vacuum and then flushed with N₂ gas. Degassed MTBE (200.0 mL) was added, and the mixture was heated to 60° C. and stirred for 6 h. The mixture was diluted with EtOAc (50.0 mL) and saturated aq NaHCO₃ (100 mL). The aq phase was extracted with EtOAc (3×75.0 mL), and the combined organic phases were washed with brine (100 mL), dried over MgSO₄, filtered, and concentrated under reduced pressure. The product was purified by flash chromatography on silica gel, eluting with mixtures of EtOAc and hexanes to provide the title compound as a solid (697 mg, 58%). ¹H NMR (500 MHz, CDCl₃) δ 8.02 (s, 1H), 7.50-7.32 (m, 5H), 6.63 (s, 1H), 6.27 (q, J=7.1 Hz, 1H), 4.63 (q, J=7.1 Hz, 2H), 3.35 (s, 3H), 2.28 (s, 3H), 2.12 (s, 3H), 2.07 (d, J=7.2 Hz, 3H), 1.53 (t, J=7.1 Hz, 3H); [M+H]⁺ 488.5.

### Intermediate Step 1 Towards Example 35

### 7-(3,5-Dimethylisoxazol-4-yl)-8-methoxy-2-oxo-1-[(1R)-1-phenylethyl]oxazolo[5,4-c]quinoline-4-carbaldehyde

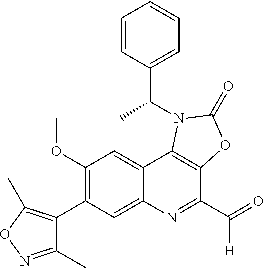 [SMILES: COc1cc2c(cc1-c1c(C)noc1C)nc(C=O)c1oc(=O)n([C@H](C)c3ccccc3)c12]

Ethyl 7-(3,5-dimethylisoxazol-4-yl)-8-methoxy-2-oxo-1-[(1R)-1-phenylethyl]oxazolo[5,4-c]quinoline-4-carboxylate (see Example 34, 2.49 g, 5.11 mmol) was dissolved in dry THF (40.0 mL), and the mixture was cooled to −78° C. DIBAL-H (15.3 mL, 15.3 mmol, 1.00M in toluene) was slowly added, and the mixture was stirred for 4 h at −78° C. The reaction was diluted with EtOH (10.0 mL) at −78° C., and the mixture was stirred for 10 m. The mixture was diluted with saturated aq Na/K tartrate (100 mL) and stirred for 2 h at rt. The organic phase was extracted with EtOAc (3×100 mL), dried over MgSO₄, filtered, and concentrated under reduced pressure. The product was purified by flash chromatography on silica gel, eluting with mixtures of EtOAc and hexanes to provide the title compound as a solid (2.26 g, 99%). ¹H NMR (500 MHz, CDCl₃) δ 10.21 (s, 1H), 7.92 (s, 1H), 7.43-7.38 (m, 4H), 7.38-7.31 (m, 1H), 6.64 (s, 1H), 6.22 (q, J=7.2 Hz, 1H), 3.37 (s, 3H), 2.27 (s, 3H), 2.10 (s, 3H), 2.06 (d, J=7.2 Hz, 3H); [M−H]+ 444.4.

### Example 35

### 7-(3,5-Dimethylisoxazol-4-yl)-4-(hydroxymethyl)-8-methoxy-1-[(1R)-1-phenylethyl]oxazolo[5,4-c]quinolin-2-one

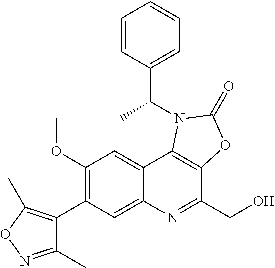 [SMILES: COc1cc2c(cc1-c1c(C)noc1C)nc(CO)c1oc(=O)n([C@H](C)c3ccccc3)c12]

NaBH(OAc)₃ (453 mg, 2.14 mmol) was added to a solution of 7-(3,5-dimethylisoxazol-4-yl)-8-methoxy-2-oxo-1-[(1R)-1-phenylethyl]oxazolo[5,4-c]quinoline-4-carbaldehyde (611 mg, 1.38 mmol) in DCE (13.0 mL). The mixture was stirred at rt for 16 h. The mixture was diluted with saturated aq NaHCO₃ (100 mL) and DCM (100 mL). The aq phase was extracted with DCM (3×75.0 mL), and the combined organic phases were washed with brine, dried over MgSO₄, filtered, and concentrated under reduced pressure. The product was purified by flash chromatography on silica gel, eluting with mixtures of DCM and MeOH to provide the title compound as a solid (586 mg, 96%). ¹H NMR (500 MHz, CDCl₃) δ 7.83 (s, 1H), 7.45-7.40 (m, 4H), 7.37 (ddd, J=8.0, 7.0, 1.7 Hz, 1H), 6.61 (s, 1H), 6.23 (q, J=7.2 Hz, 1H), 5.08 (dd, J=5.0, 1.4 Hz, 2H), 3.98 (t, J=5.0 Hz, 1H), 3.34 (s, 3H), 2.30 (s, 3H), 2.13 (s, 3H), 2.07 (d, J=7.2 Hz, 3H); [M+H]⁺ 446.3.

### Example 36

### 7-(3,5-Dimethylisoxazol-4-yl)-8-methoxy-4-methyl-1-[(1R)-1-phenylethyl]oxazolo[5,4-c]quinolin-2-one

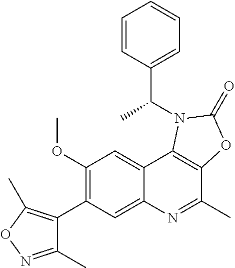 [SMILES: COc1cc2c(cc1-c1c(C)noc1C)nc(C)c1oc(=O)n([C@H](C)c3ccccc3)c12]

A mixture of imidazole (18.9 mg, 0.28 mmol) and iodine (70.2 mg, 0.28 mmol) in DCM (10.0 mL) was cooled to 0° C., and PPh₃ (72.6 mg, 0.280 mmol) was added. The mixture was stirred at 0° C. for 20 m, and 7-(3,5-dimethylisoxazol-4-yl)-4-(hydroxymethyl)-8-methoxy-1-[(1R)-1-phenylethyl]oxazolo[5,4-c]quinolin-2-one (see Example 35, 103 mg, 0.230 mmol) was added. The mixture was stirred at 0° C. for 2 h. Water (75.0 mL) was added, and the aq phase was extracted with EtOAc (3×75.0 mL). The combined organic phases were washed with brine (100 mL), dried over MgSO₄, filtered, and concentrated under reduced pressure. The product was purified by flash chromatography on silica gel, eluting with mixtures of EtOAc and hexanes to provide the title compound as a solid (40.0 mg, 40%). ¹H NMR (500 MHz, CDCl₃) δ 7.76 (s, 1H), 7.45-7.38 (m, 4H), 7.38-7.31 (m, 1H), 6.57 (s, 1H), 6.22 (q, J=7.2 Hz, 1H), 3.32 (s, 3H), 2.80 (s, 3H), 2.28 (s, 3H), 2.12 (s, 3H), 2.06 (d, J=7.2 Hz, 3H); [M+H]⁺ 430.4.

### Example 37

### 7-(3,5-Dimethylisoxazol-4-yl)-1-[(2-fluoro-6-methyl-phenyl)methyl]-8-methoxy-oxazolo[5,4-c]quinolin-2-one

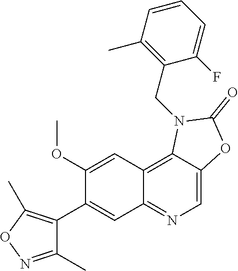 [SMILES: COc1cc2c(cc1-c1c(C)noc1C)ncc1oc(=O)n(Cc3c(C)cccc3F)c12]

A mixture of 7-(3,5-dimethylisoxazol-4-yl)-8-methoxy-1H-oxazolo[5,4-c]quinolin-2-one (see Example 11, 27.5 mg, 0.0900 mmol), Cs₂CO₃ (57.6 mg, 0.180 mmol), TBAI (32.6 mg, 0.0900 mmol), and 2-(bromomethyl)-1-fluoro-3-methyl-benzene (35.9 mg, 0.180 mmol) in dry DMF (5.00 mL) was stirred at rt for 48 h. The mixture was concentrated under reduced pressure, and the residue was diluted with EtOAc (75.0 mL) and saturated aq NaHCO₃ (75.0 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) followed by flash chromatography on silica gel, eluting with mixtures of hexanes and EtOAc to provide the title compound as a solid (7.39 mg). ¹H NMR (500 MHz, CDCl₃) δ 8.84 (s, 1H), 7.89 (s, 1H), 7.29 (dd, J=8.4, 5.6 Hz, 1H), 6.96 (td, J=8.3, 2.6 Hz, 1H), 6.71 (dd, J=9.4, 2.6 Hz, 1H), 6.67 (s, 1H), 5.44 (s, 2H), 3.41 (s, 3H), 2.47 (s, 3H), 2.31 (s, 3H), 2.14 (s, 3H); [M+H]⁺ 434.4.

### Example 38

### 7-(3,5-Dimethylisoxazol-4-yl)-8-methoxy-4-(methoxymethyl)-1-[(1R)-1-phenylethyl]oxazolo[5,4-c]quinolin-2-one

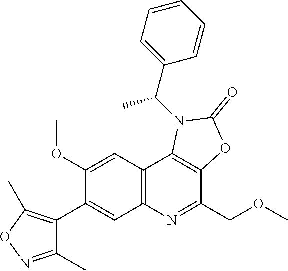 [SMILES: COCc1nc2cc(-c3c(C)noc3C)c(OC)cc2c2c1oc(=O)n2[C@H](C)c1ccccc1]

A mixture of 7-(3,5-dimethylisoxazol-4-yl)-4-(hydroxymethyl)-8-methoxy-1-[(1R)-1-phenylethyl]oxazolo[5,4-c]quinolin-2-one (see Example 35, 33.0 mg, 0.0700 mmol) and MsCl (11.5 uL) in DCM (5.00 mL) was cooled to 0° C., and TEA (21.0 uL, 0.150 mmol) was added. The mixture was stirred until starting material was not visible by TLC (˜30 m). The mixture was concentrated under reduced pressure and diluted with MeOH (10.0 mL). DMAP (9.05 mg, 0.0700 mmol) was added, and the mixture was heated to 85° C. for 16 h. The mixture was cooled to rt and concentrated under reduced pressure. The residue was diluted with EtOAc (75.0 mL) and saturated aq NaHCO₃ (75.0 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) to provide the title compound as a solid (3.75 mg, 11%). ¹H NMR (500 MHz, CDCl₃) δ 7.88 (s, 1H), 7.47-7.39 (m, 4H), 7.36 (m, 1H), 6.58 (s, 1H), 6.23 (q, J=7.2 Hz, 1H), 4.89 (s, 2H), 3.60 (s, 3H), 3.33 (s, 3H), 2.28 (s, 3H), 2.12 (s, 3H), 2.06 (d, J=7.2 Hz, 3H); [M−H]⁺ 460.3.

### Intermediate Step 1 Towards Example 39

### 33-[(1R)-1-Phenylpropyl]oxazolidine-2,4-dione

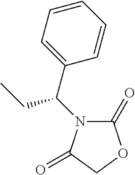 [SMILES: CC[C@H](c1ccccc1)N1C(=O)COC1=O]

A mixture of (1R)-1-phenylpropan-1-amine (0.532 mL, 3.698 mmol), ethylglycolate (0.350 mL, 3.69 mmol) and NaOMe (0.160 mL, 0.74 mmol, 4.6M in MeOH) was stirred at 120° C. for 2 h. The mixture was cooled to rt and concentrated under reduced pressure. The residue was dissolved in DMF (50.0 mL), and CDI (660 mg, 4.07 mmol) was added. The mixture was stirred at rt for 16 h and then concentrated under reduced pressure. The residue was diluted with brine (70.0 mL) and EtOAc (70.0 mL). The aq phase was extracted with EtOAc (3×70.0 mL), and the combined organic phases were washed with brine (3×70 mL), dried over MgSO₄, filtered, and concentrated under reduced pressure to provide the title compound as a solid. ¹H NMR (500 MHz, CDCl₃) δ 7.50 (dd, J=8.1, 1.4 Hz, 2H), 7.39-7.30 (m, 3H), 4.99 (dd, J=9.1, 7.3 Hz, 1H), 4.61 (s, 2H), 2.54-2.41 (m, 1H), 2.36-2.23 (m, 1H), 0.96 (t, J=7.4 Hz, 3H).

### Intermediate Step 2 Towards Example 39

### 3-[(1R)-1-Phenylpropyl]oxazol-2-one

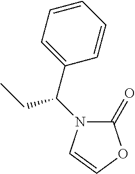

The solid from above (985.0 mg, 3.594 mmol) was dissolved in MeOH (110 mL), and the mixture was cooled to 0° C. NaBH₄ (340 mg, 8.99 mmol) was slowly added over 1.5 h followed by acetone (10.0 mL). The mixture was stirred for 5 m and then concentrated under reduced pressure. The residue was dissolved in DCM (60.0 mL) and water (50.0 mL), and the aq phase was extracted with DCM (3×60.0 mL). The combined organic phases were dried over Na₂SO₄, filtered, and concentrated under reduced pressure. The residue was dissolved in dry DCM (20.0 mL). The mixture was cooled at 0° C. TEA (0.751 mL, 5.391 mmol) and MsCl (0.556 mL, 7.189 mmol) were added, and the mixture was warmed to rt and stirred for 17 h. The mixture was diluted with saturated aq NH₄Cl (50.0 mL) and DCM (50.0 mL). The aq phase was extracted with DCM (3×50 mL), and the combined organic phases were dried over Na₂SO₄, filtered, and concentrated under reduced pressure. The product was purified by flash chromatography on silica gel, eluting with mixtures of EtOAc and hexanes to provide the title compound as an oil (306.2 mg, 42%). ¹H NMR (500 MHz, CDCl₃) δ 7.39-7.34 (m, 2H), 7.31 (d, J=6.8 Hz, 3H), 6.77 (s, 1H), 6.52 (s, 1H), 5.07-4.98 (m, 1H), 2.19-2.00 (m, 2H), 0.98 (t, J=7.3 Hz, 3H).

### Example 39

### 7-(3,5-Dimethylisoxazol-4-yl)-8-methoxy-1-[(1R)-1-phenylpropyl]oxazolo[5,4-c]quinolin-2-one

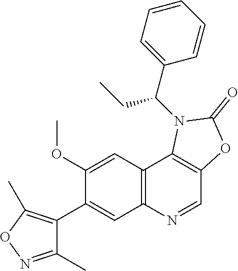 [SMILES: CC[C@H](c1ccccc1)n1c(=O)oc2cnc3cc(-c4c(C)noc4C)c(OC)cc3c21]

A mixture of 3-(3,5-dimethylisoxazol-4-yl)aniline (see Intermediate steps 4 and 5, Example 1, 100 mg, 0.458 mmol), MgSO₄ (2.21 g, 18.3 mmol), Sc(OTf)₃ (68.0 mg, 0.137 mmol), and paraformaldehyde (15.1 mg, 0.504 mmol) in dry MeCN (15.0 mL) was stirred at rt for 30 m and then added to another mixture of 3-[(1R)-1-phenylpropyl]oxazol-2-one (112 mg, 0.55 mmol), Sc(OTf)₃ (158 mg, 0.321 mmol), and 4 Å molecular sieves (1.00 g) in dry MeCN (10.0 mL). The residual MgSO₄ in the first flask was rinsed with dry MeCN (15.0 mL) and transferred to the second. The resulting mixture was heated to 50° C. and stirred for 16 h. The mixture was filtered through a pad of Celite®, washing with EtOAc. The mixture was concentrated under reduced pressure, and the residue was diluted with EtOAc (100 mL) and saturated aq NaHCO₃ (100 mL). The aq phase was extracted with EtOAc (3×50.0 mL), and the combined organic phases were washed with brine (100 mL), dried over MgSO₄, filtered, and concentrated under reduced pressure to provide 7-(3,5-dimethylisoxazol-4-yl)-8-methoxy-1-[(1R)-1-phenylpropyl]-3a,4,5,9b-tetrahydrooxazolo[5,4-c]quinolin-2-one as a solid. [M+H]⁺ 434.6. DDQ (219 mg, 0.964 mmol) and the solid from above (199 mg, 0.459 mmol) were weighed into a dry flask, and the flask was dried under high vacuum and then flushed with N₂ gas. Degassed MTBE (40.0 mL) was added, and the mixture was heated to 60° C. and stirred for 6 h. The mixture was concentrated under reduced pressure, and the residue was diluted with EtOAc (100 mL) and saturated aq NaHCO₃ (100 mL). The aq phase was extracted with EtOAc (3×75.0 mL), and the combined organic phases were washed with brine (100 mL), dried over MgSO₄, filtered, and concentrated under reduced pressure. The product was purified by flash chromatography on silica gel, eluting with mixtures of EtOAc and hexanes, followed by HPLC (high pH) to provide the title compound as a solid (74.2 mg, 38%). ¹H NMR (500 MHz, CDCl₃) δ 8.82 (s, 1H), 7.85 (s, 1H), 7.53-7.30 (m, 5H), 6.63 (s, 1H), 5.97 (d, J=8.3 Hz, 1H), 3.35 (s, 3H), 2.72 (br s, 1H), 2.41 (br s, 1H), 2.29 (s, 3H), 2.13 (s, 3H), 1.01 (t, J=7.3 Hz, 3H); [M+H]⁺ 430.6.

### Intermediate 1 Towards Example 40

### 3-[(1R)-1-(4-Chlorophenyl)ethyl]oxazolidine-2,4-dione

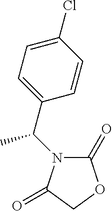 [SMILES: C[C@H](c1ccc(Cl)cc1)N1C(=O)COC1=O]

A mixture of (1R)-1-(4-chlorophenyl)ethanamine (797 mg, 5.12 mmol), ethyl glycolate (533 mg, 0.485 mL, 5.12 mmol), and NaOMe (0.222 mL, 1.02 mmol, 4.6M in MeOH) was stirred at 120° C. for 2 h. The mixture was cooled to rt and concentrated under reduced pressure. The residue was diluted with toluene and concentrated under reduced pressure. The residue was dissolved in DMF (50.0 mL), and CDI (913 mg, 5.63 mmol) was added. The mixture was stirred at rt for 2 h. The mixture diluted with brine (100 mL). The aq phase was extracted into EtOAc (3×100 mL). The combined organic phases were washed with brine (3×100 mL), dried over MgSO₄, filtered, and concentrated under reduced pressure to provide the title compound as solid (1.20 g, 98%). ¹H NMR (500 MHz, CDCl₃) δ 7.42 (d, J=8.4 Hz, 2H), 7.32 (d, J=8.5 Hz, 2H), 5.29 (q, J=7.3 Hz, 1H), 4.72-4.52 (m, 2H), 1.85 (d, J=7.4 Hz, 3H).

### Intermediate 2 Towards Example 40

### 3-[(1R)-1-(4-chlorophenyl)ethyl]oxazol-2-one

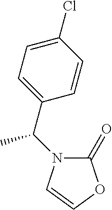

A mixture of 3-[(1R)-1-(4-chlorophenyl)ethyl]oxazolidine-2,4-dione (1.20 g, 5.00 mmol) in MeOH (150 mL) was cooled to 0° C., and NaBH₄ (568 mg, 15.0 mmol) was slowly added in portions over 2 h. Acetone (15.0 mL) was slowly added, and the mixture was slowly warmed to rt. The mixture was concentrated under reduced pressure, and the residue was diluted with brine (100 mL) and EtOAc (100 mL). The aq phase was extracted with EtOAc (3×100 mL), and the combined organic phases were dried over MgSO₄, filtered, and concentrated under reduced pressure. The residue (1.21 g, 5.00 mmol) was dissolved in DCM (50.0 mL), and TEA (6.98 mL, 50.1 mmol) and MsCl (0.426 mL, 5.51 mmol) were added. The mixture was stirred for 2 h and then diluted with brine (100 mL). The aq phase was extracted with EtOAc (3×75.0 mL), and the combined organic phases were dried over MgSO₄, filtered, and concentrated under reduced pressure. The product was purified by flash chromatography on silica gel, eluting with mixtures of EtOAc and hexanes to provide the title compound as a solid (0.486 g, 43%). ¹H NMR (500 MHz, CDCl₃) δ 7.30 (d, J=8.5 Hz, 2H), 7.22 (d, J=8.4 Hz, 2H), 6.76 (d, J=2.0 Hz, 1H), 6.46 (d, J=2.1 Hz, 1H), 5.25 (q, J=7.1 Hz, 1H), 1.65 (d, J=7.1 Hz, 3H).

### Example 40

### 1-[(1R)-1-(4-Chlorophenyl)ethyl]-7-(3,5-dimethylisoxazol-4-yl)-8-methoxy-oxazolo[5,4-c]quinolin-2-one

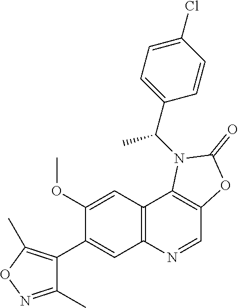 [SMILES: COc1cc2c(cc1-c1c(C)noc1C)ncc1oc(=O)n([C@H](C)c3ccc(Cl)cc3)c12]

A mixture of 3-(3,5-dimethylisoxazol-4-yl)-4-methoxy-aniline (see Intermediate steps 4 and 5, Example 1, 117 mg, 0.535 mmol), MgSO₄ (2.58 g, 21.4 mmol), Sc(OTf)₃ (79.0 mg, 0.161 mmol), and paraformaldehyde (17.7 mg, 0.589 mmol) in dry MeCN (5.00 mL) was stirred at rt for 1 h and then added to another mixture of 3-[(1R)-1-(4-chlorophenyl)ethyl]oxazol-2-one (144 mg, 0.642 mmol), Sc(OTf)₃ (184 mg, 0.375 mmol), and 4 Å molecular sieves (1.00 g) in dry MeCN (5.00 mL). The residual MgSO₄ in the first flask was rinsed with dry MeCN (10.0 mL) and transferred to the second. The resulting mixture was heated to 50° C. and stirred for 16 h. The mixture was filtered through a pad of Celite®, washing with EtOAc. The filtrate was concentrated under reduced pressure and diluted with saturated aq NaHCO₃ (100 mL) and EtOAc (100 mL). The aq phase was extracted with EtOAc (3×100 mL). The combined organic fractions were washed with brine (100 mL), dried over MgSO₄, filtered, and concentrated under reduced pressure to provide 1-[(1R)-1-(4-chlorophenyl)ethyl]-7-(3,5-dimethylisoxazol-4-yl)-8-methoxy-3a,4,5,9b-tetrahydrooxazolo[5,4-c]quinolin-2-one as a solid (243 mg, 100%). [M+H]⁺ 454.5. DDQ (255 mg, 1.12 mmol) and the solid from above (243 m g, 0.535 mmol) were weighed into a dry flask, and the flask was dried under high vacuum and then flushed with N₂ gas. Degassed MBTE (10.0 mL) was added, and the mixture was heated to 60° C. and stirred for 4 h. The mixture was concentrated under reduced pressure. The product was purified by flash chromatography on silica gel, eluting with mixtures of EtOAc and hexanes to provide the title compound as a solid (71.3 mg, 30%). ¹H NMR (500 MHz, CDCl₃) δ 8.81 (s, 1H), 7.86 (s, 1H), 7.50-7.32 (m, 4H), 6.58 (s, 1H), 6.17 (q, J=7.2 Hz, 1H), 3.41 (s, 3H), 2.30 (s, 3H), 2.14 (s, 3H), 2.06 (d, J=7.2 Hz, 3H); [M+H]⁺ 450.

### Example 41

### 1-Benzyl-7-(3,5-dimethylisoxazol-4-yl)-8-methoxy-4-(morpholinomethyl)oxazolo[5,4-c]quinolin-2-one

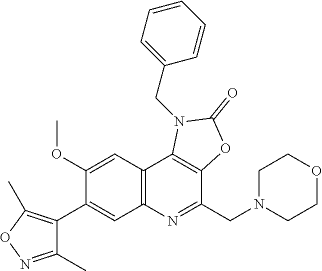 [SMILES: COc1cc2c(cc1-c1c(C)noc1C)nc(CN1CCOCC1)c1oc(=O)n(Cc3ccccc3)c12]

NaBH(OAc)₃ (10.0 mg, 0.0470 mmol) was added to a mixture of 1-benzyl-7-(3,5-dimethylisoxazol-4-yl)-8-methoxy-2-oxo-oxazolo[5,4-c]quinoline-4-carbaldehyde (see Intermediate step 1, Example 33, 8.10 mg, 0.0190 mmol) and morpholine (2.00 mg 0.0230 mmol,) in DCE (2.00 mL). The mixture was stirred at rt for 12 h. Saturated aq NaHCO₃ (5.00 mL) was added, and the mixture was extracted with DCM (3×10.0 mL). The combined organic phases were dried over Na₂SO₄ and concentrated under reduced pressure. The product was purified by HPLC (low pH) to provide the title compound as a solid (1.20 mg, 13%). ¹H NMR (300 MHz, MeOD) δ 7.96 (s, 1H), 7.49-7.33 (m, 5H), 7.19 (s, 1H), 5.67 (s, 2H), 4.90 (s, 2H), 4.05-3.98 (m, 4H), 3.65 (s, 3H), 3.63-2.29 (m, 4H), 2.29 (s, 3H), 2.10 (s, 3H); [M+H]⁺ 501.4.

### Example 42

### 4-(Dimethylaminomethyl)-7-(3,5-dimethylisoxazol-4-yl)-8-methoxy-1-[(1R)-1-phenylethyl]oxazolo[5,4-c]quinolin-2-one

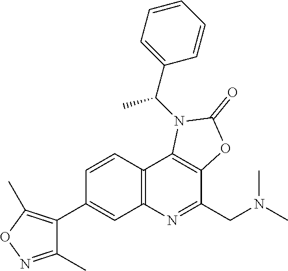 [SMILES: Cc1noc(C)c1-c1ccc2c(c1)nc(CN(C)C)c1oc(=O)n([C@H](C)c3ccccc3)c12]

A mixture of 7-(3,5-dimethylisoxazol-4-yl)-8-methoxy-2-oxo-1-[(1R)-1-phenylethyl]-3H-imidazo[4,5-c]quinoline-4-carbaldehyde (see Intermediate step 1, Example 35, 25.6 mg, 0.0580 mmol), 4 Å molecular sieves (100 mg), N-methylmethanamine (0.058 mL, 0.115 mmol, 2M in THF) in DCE (5.00 mL) was stirred at rt for 2 h. NaBH(OAc)₃ (36.7 mg, 0.173 mmol) was added, and the mixture was stirred at rt for 12 h. The mixture was filtered, and the filtrate was diluted with saturated aq NaHCO₃ (25.0 mL) and DCM (25.0 mL). The aq phase was extracted with DCM (3×25.0 mL), and the combined organic phases were washed with brine (50 mL), dried over MgSO₄, filtered, and concentrated under reduced pressure. The product was purified by HPLC (high pH) to provide the title compound as a solid (18.4 mg, 67%). ¹H NMR (500 MHz, MeOD) δ 7.95 (s, 1H), 7.55 (d, J=8.1 Hz, 2H), 7.44 (t, J=7.6 Hz, 2H), 7.37 (t, J=7.2 Hz, 1H), 6.78 (s, 1H), 6.24 (q, J=7.1 Hz, 1H), 3.45 (s, 3H), 3.13 (s, 6H), 2.27 (s, 3H), 2.11 (d, J=7.2 Hz, 3H), 2.08 (s, 3H); [M+H]⁺ 473.4.

### Intermediate Step 1 Towards Example 43

### tert-Butyl-[(E)-(4-cyanophenyl)methyleneamino]-oxido-sulfonium

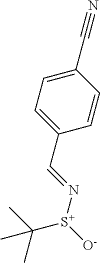 [SMILES: CC(C)(C)[S+]([O-])/N=C/c1ccc(C#N)cc1]

KHSO₄ (4.09 g, 30.0 mmol) was added to a mixture of (S)-(+)-2-methyl-2-propanesulfinamide (2.00 g, 16.5 mmol), 4 Å molecular sieves (2.00 g), and 4-formylbenzonitrile (2.38 g, 18.2 mmol) in toluene (150 mL). The mixture was heated to 45° C. and stirred for 48 h. The mixture was cooled to rt and filtered through a pad of Celite®. The filtrate was concentrated under reduced pressure. The product was purified by flash chromatography on silica gel, eluting with mixtures of hexanes and EtOAc to provide the title compound as a solid (2.66 g, 69%). ¹H NMR (500 MHz, CDCl₃) δ 8.62 (s, 1H), 7.95 (d, J=8.3 Hz, 2H), 7.77 (d, J=8.3 Hz, 2H), 1.28 (s, 9H); [M+H]⁺ 235.4.

### Intermediate Step 2 Towards Example 43

### tert-Butyl-[[(1R)-1-(4-cyanophenyl)ethyl]amino]-oxido-sulfonium

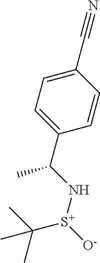 [SMILES: C[C@@H](N[S+]([O-])C(C)(C)C)c1ccc(C#N)cc1]

A mixture of tert-butyl-[(E)-(4-cyanophenyl)methyleneamino]-oxido-sulfonium (2.66 g, 11.3 mmol) in DCM (65.0 mL) was cooled to −45° C. MeMgBr (9.45 mL, 28.4 mmol) was slowly added over 30 m. The mixture was stirred at −45° C. for 4 h and then warmed to −10° C. The mixture was diluted with saturated aq NaHCO₃ (100 mL), and the aq phase was extracted with DCM (3×75.0 mL). The combined organic phases were washed with brine (100 mL), dried over MgSO₄, filtered, and concentrated under reduced pressure to provide the title compound as a solid. ¹H NMR (500 MHz, CDCl₃) δ 7.63 (d, J=8.4 Hz, 2H), 7.44 (d, J=8.1 Hz, 2H), 4.62 (qd, J=6.7, 3.0 Hz, 1H), 3.20 (s, 1H), 1.52 (d, J=6.7 Hz, 3H), 1.20 (s, 9H); [M+H]⁺ 251.4.

### Intermediate Step 3 Towards Example 43

### 4-[(1R)-1-Aminoethyl]benzonitrile

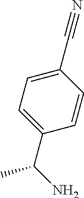

tert-Butyl-[[(1R)-1-(4-cyanophenyl)ethyl]amino]-oxido-sulfonium (2.84 g, 11.3 mmol) was dissolved in MeOH (50.0 mL) and HCl (5.67 mL, 22.7 mmol, 4M in dioxane) was added. The mixture was stirred for 60 m and concentrated under reduced pressure. The residue was triturated with Et₂O, and the solid was recrystallized from MTBE and EtOH to provide 4-[(1R)-1-aminoethyl]benzonitrile hydrochloride as a solid (1.43 g, 69%). The solid was diluted with DCM (75.0 mL) and saturated aq NaHCO₃ (100 mL), and the aq phase was extracted with DCM (3×75.0 mL). The combined organic phases were dried over MgSO₄, filtered, and concentrated under reduced pressure to provide the title compound as a liquid (1.06 g, 64%). ¹H NMR (500 MHz, CDCl₃) δ 7.64-7.51 (m, 2H), 7.43 (d, J=8.1 Hz, 2H), 4.15 (q, J=6.6 Hz, 1H), 1.33 (d, J=6.6 Hz, 3H).

### Intermediate Step 4 Towards Example 43

### 4-[(1R)-1-(2,4-Dioxooxazolidin-3-yl)ethyl]benzonitrile

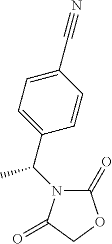 [SMILES: C[C@H](c1ccc(C#N)cc1)N1C(=O)COC1=O]

A mixture of 4-[(1R)-1-amino ethyl]benzonitrile hydrochloride (1.06 g, 5.81 mmol), ethyl glycolate (605 mg, 0.550 mL, 5.81 mmol), and NaOMe (0.126 mL, 0.581 mmol) was stirred at 120° C. for 2 h. The mixture was cooled to rt and concentrated under reduced pressure. The residue was diluted with toluene and concentrated under reduced pressure. The residue was dissolved in DMF (30.0 mL), and CDI (1.04 g, 6.40 mmol) was added. The mixture was stirred at rt for 2 h. The mixture diluted with brine (100 mL). The aq phase was extracted into EtOAc (3×100 mL). The combined organic phases were washed with brine (3×100 mL), dried over MgSO₄, filtered, and concentrated under reduced pressure. The product was purified by flash chromatography on silica gel, eluting with mixtures of hexanes and EtOAc to provide the title compound as solid (804 mg, 60%).

### Intermediate Step 5 Towards Example 43

### 3-[(1R)-1-(2-Oxooxazol-3-yl)ethyl]benzonitrile

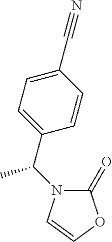 [SMILES: C[C@H](c1ccc(C#N)cc1)n1ccoc1=O]

A solution of 3-[(1R)-1-(2,4-dioxooxazolidin-3-yl)ethyl]benzonitrile (732 mg, 3.18 mmol) in MeOH (50.0 mL) was cooled to 0° C., and NaBH₄ (300 mg, 7.95 mmol) was slowly added over 2 h. Acetone (15.0 mL) was slowly added, and the mixture was stirred for 5 m. The mixture was concentrated under reduced pressure, and the residue was diluted with DCM (100 mL). The mixture was filtered over Celite®, washing with DCM. The filtrate was diluted with saturated aq NH₄Cl (100 mL), and the aq phase was extracted with DCM (3×75.0 mL). The combined organic phases were dried over MgSO₄, filtered, and concentrated under reduced pressure. The residue was dissolved in dry DCM (75.0 mL), and the mixture was cooled at 0° C. TEA (0.665 mL, 4.77 mmol) and MsCl (0.492 mL, 6.36 mmol) were added. The mixture was warmed to rt and stirred for 6 h. The mixture was diluted with saturated aq NaHCO₃ (75.0 mL), and the aq phase was extracted with DCM (3×75.0 mL). The combined organic phases were dried over MgSO₄, filtered, and concentrated under reduced pressure. The product was purified by flash chromatography on silica gel, eluting with mixtures of hexanes and EtOAc to provide the title compound as a solid (419 mg, 61%). ¹H NMR (500 MHz, CDCl₃) δ 7.60 (dt, J=7.5, 1.3 Hz, 1H), 7.57 (t, J=1.7 Hz, 1H), 7.56-7.53 (m, 1H), 7.48 (t, J=7.7 Hz, 1H), 6.83 (d, J=2.1 Hz, 1H), 6.54 (d, J=2.1 Hz, 1H), 5.31 (q, J=7.2 Hz, 1H), 1.71 (d, J=7.2 Hz, 3H); [M+H]⁺ 215.3.

### Intermediate Step 6 Towards Example 43

### 4-[(1R)-1-[7-(3,5-Dimethylisoxazol-4-yl)-8-methoxy-2-oxo-3a,4,5,9b-tetrahydrooxazolo[5,4-c]quinolin-1-yl]ethyl]benzonitrile

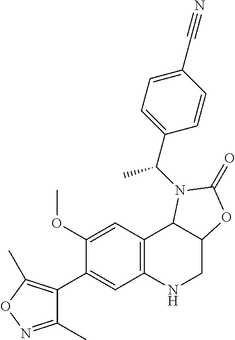 [SMILES: COc1cc2c(cc1-c1c(C)noc1C)NCC1OC(=O)N([C@H](C)c3ccc(C#N)cc3)C21]

Paraformaldehyde (11.4 mg, 0.380 mmol), 3-(3,5-dimethylisoxazol-4-yl)-4-methoxy-aniline (see Intermediate steps 4 and 5, Example 1, 75.0 mg, 0.340 mmol), MgSO₄ (1.65 g, 13.8 mmol), and Sc(OTf)₃ (50.7 g, 0.100 mmol) were weighed into a dry flask, and dry MeCN (10.0 mL) was added. The mixture was stirred at rt for 1 h and then added to another mixture of 4-[(1R)-1-(2-oxooxazol-3-yl)ethyl]benzonitrile (88.3 g, 0.410 mmol), Sc(OTf)₃ (118 mg, 0.240 mmol), and 4 Å molecular sieves (200 mg). The residual MgSO₄ in the first flask was rinsed with dry MeCN (10.0 mL), and the liquid was transferred to the second. The resulting mixture was heated to 50° C. and stirred for 16 h. The mixture was filtered through a pad of Celite®, washing with EtOAc. The mixture was concentrated under reduced pressure, and the residue was diluted with saturated aq NaHCO₃ (100 mL) and EtOAc (100 mL). The aq phase was extracted with EtOAc (3×100 mL), and the combined organic phases were dried over MgSO₄, filtered, and concentrated under reduced pressure to the title compound as a solid. [M+H]+ 445.3.

### Example 43

### 4-[(1R)-1-[7-(3,5-Dimethylisoxazol-4-yl)-8-methoxy-2-oxo-oxazolo[5,4-c]quinolin-1-yl]ethyl]benzonitrile

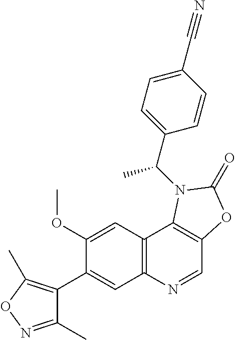 [SMILES: COc1cc2c(cc1-c1c(C)noc1C)ncc1oc(=O)n([C@H](C)c3ccc(C#N)cc3)c12]

DDQ (172 mg, 0.760 mmol) and 4-[(1R)-1-[7-(3,5-dimethylisoxazol-4-yl)-8-methoxy-2-oxo-3a,4,5,9b-tetrahydrooxazolo[5,4-c]quinolin-1-yl]ethyl]benzonitrile (150 mg, 0.340 mmol) were weighed into a dry flask equipped with a reflux condenser, and the flask was dried under high vacuum and then flushed with N₂ gas. Degassed MTBE (10.0 mL) was added, and the mixture was heated to 60° C. and stirred for 6 h. The mixture was cooled to rt and filtered through a pad of Celite®, washing with EtOAc. The filtrate was concentrated under reduced pressure. The residue was diluted with EtOAc (75.0 mL) and saturated aq NaHCO₃ (100 mL). The aqueous phase was extracted with EtOAc (3×75.0 mL), and the combined organic phases were washed with brine (75.0 mL), dried over MgSO₄, filtered, and concentrated under reduced pressure. The product was purified by flash chromatography on silica gel, eluting with mixtures of EtOAc and hexanes, to provide the title compound as a solid (20.9 mg, 14%). ¹H NMR (500 MHz, MeOD) δ 9.27 (s, 1H), 8.01 (s, 1H), 7.92-7.77 (m, 4H), 6.94 (s, 1H), 6.46 (q, J=7.4 Hz, 1H), 3.62 (s, 3H), 2.35 (s, 3H), 2.20 (d, J=7.2 Hz, 3H), 2.16 (s, 3H); [M+H]⁺ 441.5.

### Intermediate 1 Towards Example 44

### tert-Butyl-[(E)-(3-cyanophenyl)methyleneamino]-oxido-sulfonium

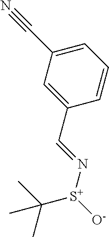 [SMILES: CC(C)(C)[S+]([O-])/N=C/c1cccc(C#N)c1]

KHSO₄ (4.09 g, 30.0 mmol) was added to a mixture of (S)-(+)-2-methyl-2-propanesulfinamide (2.00 g, 16.5 mmol), 4 Å molecular sieves (2.00 g), and 3-formylbenzonitrile (2.38 g, 18.2 mmol) in toluene (150 mL). The mixture was heated to 45° C. and stirred for 60 h. The mixture was cooled to rt and filtered through a pad of Celite®. The filtrate was concentrated under reduced pressure to provide the title compound as a solid (3.14 g, 81%). ¹H NMR (500 MHz, CDCl₃) δ 8.57 (s, 1H), 8.15 (t, J=1.5 Hz, 1H), 8.03 (dt, J=7.9, 1.4 Hz, 1H), 7.80-7.74 (m, 1H), 7.61 (t, J=7.8 Hz, 1H), 1.26 (s, 9H); [M+H]⁺ 235.4.

### Intermediate 2 Towards Example 44

### tert-Butyl-[[(1R)-1-(3-cyanophenyl)ethyl]amino]-oxido-sulfonium

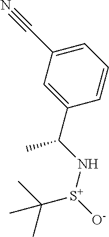 [SMILES: C[C@@H](N[S+]([O-])C(C)(C)C)c1cccc(C#N)c1]

A mixture of tert-butyl-[(E)-(3-cyanophenyl)methyleneamino]-oxido-sulfonium (3.14 g, 13.4 mmol) in DCM (65.0 mL) was cooled to −45° C. MeMgBr (11.2 mL, 33.5 mmol) was slowly added over 30 m. The mixture was stirred at −45° C. for 4 h and then warmed to −10° C. The mixture was diluted with saturated aq NaHCO₃ (100 mL), and the aq phase was extracted with DCM (3×75.0 mL). The combined organic phases were washed with brine (100 mL), dried over MgSO₄, filtered, and concentrated under reduced pressure to provide the title compound as a solid. ¹H NMR (500 MHz, CDCl₃) δ 7.62 (s, 1H), 7.57-7.52 (m, 2H), 7.43 (t, J=7.6 Hz, 1H), 4.59 (qd, J=6.7, 3.1 Hz, 1H), 3.19 (s, 1H), 1.52 (d, J=6.7 Hz, 3H), 1.19 (s, 9H); [M+H]⁺ 251.5.

### Intermediate 3 Towards Example 44

### 3-[(1R)-1-Aminoethyl]benzonitrile

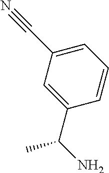

tert-Butyl-[[(1R)-1-(3-cyanophenyl)ethyl]amino]-oxido-sulfonium (3.35 g, 13.4 mmol) was dissolved in MeOH (50.0 mL) and HCl (6.69 mL, 26.8 mmol, 4M in dioxane) was added. The mixture was stirred for 60 m and concentrated under reduced pressure. The residue was triturated with Et₂O, and the solid was recrystallized from MTBE and EtOH to provide 3-[(1R)-1-aminoethyl]benzonitrile hydrochloride as a solid (1.5 g, 61%). ¹H NMR (500 MHz, MeOD) δ 7.89 (t, J=1.7 Hz, 1H), 7.82 (tt, J=7.9, 1.2 Hz, 2H), 7.68 (t, J=7.8 Hz, 1H), 4.59 (q, J=6.9 Hz, 1H), 1.68 (d, J=6.9 Hz, 3H). The solid was diluted with DCM (75.0 mL) and saturated aq NaHCO₃ (100 mL), and the aq phase was extracted with DCM (3×75.0 mL). The combined organic phases were dried over MgSO₄, filtered and concentrated under reduced pressure to provide the title compound as a liquid (1.09 g, 56%). ¹H NMR (500 MHz, CDCl₃) δ 7.62 (t, J=1.7 Hz, 1H), 7.57-7.53 (m, 1H), 7.45 (dt, J=7.7, 1.4 Hz, 1H), 7.36 (t, J=7.7 Hz, 1H), 4.12 (q, J=6.6 Hz, 1H), 1.44 (s, 2H), 1.31 (d, J=6.6 Hz, 3H).

### Intermediate 4 Towards Example 44

### 3-[(1R)-1-(2,4-Dioxooxazolidin-3-yl)ethyl]benzonitrile

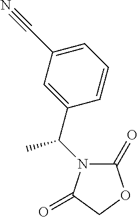 [SMILES: C[C@H](c1cccc(C#N)c1)N1C(=O)COC1=O]

A mixture of 3-[(1R)-1-aminoethyl]benzonitrile hydrochloride (1.09 g, 5.95 mmol), ethyl glycolate (619 mg, 0.563 mL, 5.95 mmol), and NaOMe (0.129 mL, 0.595 mmol) was stirred at 120° C. for 2 h. The mixture was cooled to rt and concentrated under reduced pressure. The residue was diluted with toluene and concentrated under reduced pressure. The residue was dissolved in DMF (30.0 mL), and CDI (1.06 g, 6.54 mmol) was added. The mixture was stirred at rt for 2 h. The mixture diluted with brine (100 mL). The aqueous phase was extracted with EtOAc (3×100 mL), and the combined organic phases were washed with brine (3×100 mL), dried over MgSO₄, filtered, and concentrated under reduced pressure. The product was purified by flash chromatography on silica gel, eluting with mixtures of hexanes and EtOAc to provide the title compound as a solid (732 mg, 53%).

### Intermediate 5 Towards Example 44

### 3-[(1R)-1-(2-Oxooxazol-3-yl)ethyl]benzonitrile

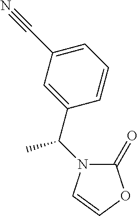 [SMILES: C[C@H](c1cccc(C#N)c1)n1ccoc1=O]

A solution of 3-[(1R)-1-(2,4-dioxooxazolidin-3-yl)ethyl]benzonitrile (732 mg, 3.18 mmol) in MeOH (50.0 mL) was cooled to 0° C., and NaBH₄ (300 mg, 7.95 mmol) was slowly added over 2 h. Acetone (15.0 mL) was slowly added, and the mixture was stirred for 5 m. The mixture was concentrated under reduced pressure, and the residue was diluted with DCM (100 mL). The mixture was filtered over Celite®, washing with DCM. The filtrate was diluted with saturated aq NH₄Cl (100 mL), and the aq phase was extracted with DCM (3×75.0 mL). The combined organic phases were dried over MgSO₄, filtered, and concentrated under reduced pressure. The residue was dissolved in dry DCM (75.0 mL), and the mixture was cooled at 0° C. TEA (0.665 mL, 4.77 mmol) and MsCl (0.492 mL, 6.36 mmol) were added. The mixture was warmed to rt and stirred for 6 h. The mixture was diluted with saturated aq NaHCO₃ (75.0 mL), and the aq phase was extracted with DCM (3×75.0 mL). The combined organic phases were dried over MgSO₄, filtered, and concentrated under reduced pressure. The product was purified by flash chromatography on silica gel, eluting with mixtures of hexanes and EtOAc to provide the title compound as a solid (419 mg, 61%). ¹H NMR (500 MHz, CDCl₃) δ 7.60 (dt, J=7.5, 1.3 Hz, 1H), 7.57 (t, J=1.7 Hz, 1H), 7.56-7.53 (m, 1H), 7.48 (t, J=7.7 Hz, 1H), 6.83 (d, J=

### Intermediate 6 Towards Example 44

### 3-[(1R)-1-[7-(3,5-Dimethylisoxazol-4-yl)-8-methoxy-2-oxo-3a,4,5,9b-tetrahydrooxazolo[5,4-c]quinolin-1-yl]ethyl]benzonitrile

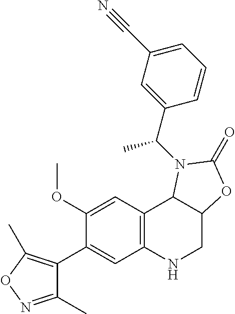 [SMILES: COc1cc2c(cc1-c1c(C)noc1C)NCC1OC(=O)N([C@H](C)c3cccc(C#N)c3)C21]

Paraformaldehyde (11.4 mg, 0.380 mmol), 3-(3,5-dimethylisoxazol-4-yl)-4-methoxy-aniline (see Intermediate steps 4 and 5, Example 1, 75.0 mg, 0.340 mmol), MgSO4 (1.65 g, 13.8 mmol), and Sc(OTf)₃ (50.7 g, 0.100 mmol) were weighed into a dry flask, and dry MeCN (10.0 mL) was added. The mixture was stirred at rt for 1 h and then added to another mixture of 3-[(1R)-1-(2-oxooxazol-3-yl)ethyl]benzonitrile (88.3 g, 0.410 mmol), Sc(OTf)₃ (118 mg, 0.240 mmol), and 4 Å molecular sieves (200 mg). The residual MgSO₄ in the first flask was rinsed with dry MeCN (10.0 mL), and the liquid was transferred to the second. The resulting mixture was heated to 50° C. and stirred for 16 h. The mixture was filtered through a pad of Celite®, washing with EtOAc. The mixture was concentrated under reduced pressure, and the residue was diluted with saturated aq NaHCO₃ (100 mL) and EtOAc (100 mL). The aq phase was extracted with EtOAc (3×100 mL), and the combined organic phases were dried over MgSO₄, filtered, and concentrated under reduced pressure to provide the title compound as a solid. [M+H]⁺ 445.3.

### Example 44

### 3-[(1R)-1-[7-(3,5-Dimethylisoxazol-4-yl)-8-methoxy-2-oxo-oxazolo[5,4-c]quinolin-1-yl]ethyl]benzonitrile

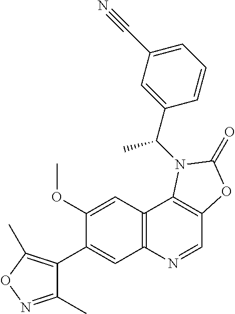 [SMILES: COc1cc2c(cc1-c1c(C)noc1C)ncc1oc(=O)n([C@H](C)c3cccc(C#N)c3)c12]

DDQ (172 g, 0.760 mmol) and 3-[(1R)-1-[7-(3,5-dimethylisoxazol-4-yl)-8-methoxy-2-oxo-3a,4,5,9b-tetrahydrooxazolo[5,4-c]quinolin-1-yl]ethyl]benzonitrile (150 mg, 0.340 mmol) were weighed into a dry flask equipped with a reflux condenser, and the flask was dried under high vacuum and then flushed with N₂ gas. Degassed MTBE (10.0 mL) was added, and the mixture was heated to 60° C. and stirred for 6 h. The mixture was cooled to rt and filtered through a pad of Celite®, washing with EtOAc. The filtrate was concentrated under reduced pressure. The residue was diluted with EtOAc (75.0 mL) and saturated aq NaHCO₃ (100 mL). The aq phase was extracted with EtOAc (3×75.0 mL), and the combined organic phases were washed with brine (75.0 mL), dried over MgSO₄, filtered, and concentrated under reduced pressure. The product was purified by flash chromatography on silica gel, eluting with mixtures of EtOAc and hexanes to provide the title compound as a solid (43.1 mg, 29%). ¹H NMR (500 MHz, MeOD) δ 9.33 (s, 1H), 8.10 (s, 1H), 8.05 (s, 1H), 8.02-7.96 (m, 1H), 7.82 (d, J=7.7 Hz, 1H), 7.70 (t, J=7.9 Hz, 1H), 7.00 (s, 1H), 6.46 (q, J=7.1 Hz, 1H), 3.66 (s, 3H), 2.36 (s, 3H), 2.21 (d, J=7.2 Hz, 3H), 2.17 (s, 3H); [M+H]⁺ 441.3.

### Intermediate Step 1 Towards Example 45

### 3-[(1R)-1-(3-Chlorophenyl)ethyl]oxazolidine-2,4-dione

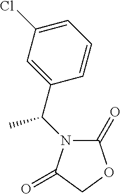 [SMILES: C[C@H](c1cccc(Cl)c1)N1C(=O)COC1=O]

A mixture of (1R)-1-(3-chlorophenyl)ethanamine (293 mg, 1.88 mmol), ethyl glycolate (196 mg, 1.88 mL, 0.178 mmol), and NaOMe (0.0820 mL, 0.377 mmol) was stirred at 120° C. for 2 h. The mixture was cooled to rt and concentrated under reduced pressure. The residue was diluted with toluene and concentrated under reduced pressure. The residue was dissolved in DMF (10.0 mL), and CDI (336 mg, 2.07 mmol) was added. The mixture was stirred at rt for 2 h. The mixture was diluted with brine (100 mL). The aq phase was extracted into EtOAc (3×100 mL), and the combined organic phases were washed with brine (3×100 mL), dried over MgSO₄, filtered, and concentrated under reduced pressure. The product was purified by flash chromatography on silica gel, eluting with mixtures of EtOAc and hexanes to provide the title compound as a solid (100 mg, 22%). ¹H NMR (500 MHz, CDCl₃) δ 7.36-7.22 (m, 3H), 7.19 (ddd, J=5.9, 2.0, 0.9 Hz, 1H), 6.80 (d, J=2.1 Hz, 1H), 6.48 (d, J=2.1 Hz, 1H), 5.30 (q, J=7.1 Hz, 1H), 1.69 (d, J=7.2 Hz, 1H).

### Intermediate Step 2 Towards Example 45

### 3-[(1R)-1-(3-Chlorophenyl)ethyl]oxazol-2-one

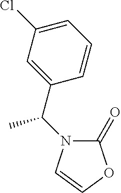

A mixture of 3-[(1R)-1-(3-chlorophenyl)ethyl]oxazolidine-2,4-dione (214.2 mg, 0.893 mmol) in MeOH (25.0 mL) was cooled to 0° C., and NaBH₄ (67.6 mg, 1.786 mmol) was slowly added over 1 h. Acetone (5.00 mL) was slowly added, and the mixture was stirred for 5 m. The mixture was concentrated under reduced pressure, and the residue was dissolved in DCM (15.0 mL) and water (10.0 mL). The aq phase was extracted with DCM (3×15.0 mL), and the combined organic phases were dried over Na₂SO₄, filtered, and concentrated under reduced pressure. The residue was dissolved in dry DCM (5.00 mL), and the mixture was cooled at 0° C. TEA (0.187 mL, 1.34 mmol) and MsCl (0.138 mL, 1.79 mmol) were added, and the mixture was warmed to rt and stirred for 17 h. The mixture was diluted with saturated aq NH₄Cl (15.0 mL), and the aq phases was extracted with DCM (3×10.0 mL). The combined organic phases were dried over Na₂SO₄, filtered, and concentrated under reduced pressure. The product was purified by flash chromatography on silica gel, eluting with mixtures of EtOAc and hexanes to provide the title compound as an oil (101 mg, 50%). ¹H NMR (500 MHz, CDCl₃) δ 7.32-7.27 (m, 3H), 7.22-7.17 (m, 1H), 6.80 (d, J=2.1 Hz, 1H), 6.48 (d, J=2.1 Hz, 1H), 5.34-5.25 (m, 1H), 1.69 (d, J=7.2 Hz, 3H).

### Intermediate Step 3 Towards Example 45

### 1-[(1R)-1-(3-Chlorophenyl)ethyl]-7-(3,5-dimethylisoxazol-4-yl)-8-methoxy-3a,4,5,9b-tetrahydrooxazolo[5,4-c]quinolin-2-one

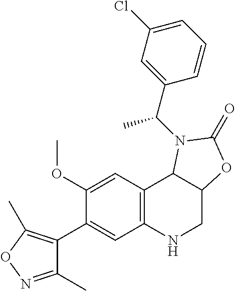 [SMILES: COc1cc2c(cc1-c1c(C)noc1C)NCC1OC(=O)N([C@H](C)c3cccc(Cl)c3)C21]

A mixture of 3-(3,5-dimethylisoxazol-4-yl)aniline (see Intermediate steps 4 and 5, Example 1, 80.0 mg, 0.367 mmol), MgSO₄ (1.77 g, 14.7 mmol), Sc(OTf)₃ (54.0 mg, 0.110 mmol), and paraformaldehyde (12.1 mg, 0.403 mmol) in dry MeCN (15.0 mL) was stirred at rt for 30 m and then added to another mixture of 3-[(1R)-1-(3-chlorophenyl)ethyl]oxazol-2-one (9.8.4 mg, 0.440 mmol), Sc(OTf)₃ (126 mg, 0.257 mmol), and 4 Å molecular sieves (1.00 g) in dry MeCN (10.0 mL). The residual MgSO₄ in the first flask was rinsed with dry MeCN (15.0 mL) and transferred to the second. The resulting mixture was heated to 50° C. and stirred for 16 h. The mixture was filtered through a pad of Celite®, washing with EtOAc. The mixture was concentrated under reduced pressure, and the residue was diluted with EtOAc (100 mL) and saturated aq NaHCO₃ (100 mL). The aq phase was extracted with EtOAc (3×50.0 mL), and the combined organic phases were washed with brine (100 mL), dried over MgSO₄, filtered, and concentrated under reduced pressure to provide the title compound as a solid. [M+H]⁺ 454.6.

### Example 45

### 1-[(1R)-1-(3-Chlorophenyl)ethyl]-7-(3,5-dimethylisoxazol-4-yl)-8-methoxy-oxazolo[5,4-c]quinolin-2-one

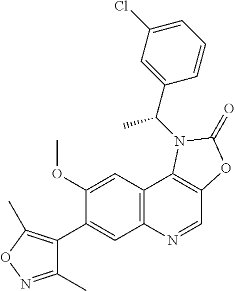 [SMILES: COc1cc2c(cc1-c1c(C)noc1C)ncc1oc(=O)n([C@H](C)c3cccc(Cl)c3)c12]

DDQ (174 mg, 0.768 mmol) and 1-[(1R)-1-(3-chlorophenyl)ethyl]-7-(3,5-dimethylisoxazol-4-yl)-8-methoxy-3a,4,5,9b-tetrahydrooxazolo[5,4-c]quinolin-2-one (166 mg, 0.366 mmol) were weighed into a dry flask, and the flask was dried under high vacuum and then flushed with N₂ gas. Degassed MTBE (40.0 mL) was added, and the mixture was heated to 60° C. and stirred for 6 h. The mixture was concentrated under reduced pressure, and the residue was diluted with EtOAc (100 mL) and saturated aq NaHCO₃ (100 mL). The aq phase was extracted with EtOAc (3×75.0 mL), and the combined organic phases were washed with brine (100 mL), dried over MgSO₄, filtered, and concentrated under reduced pressure. The product was purified by flash chromatography on silica gel, eluting with mixtures of EtOAc and hexanes, followed by HPLC (high pH) to provide the title compound as a solid (56.7 mg, 34%). ¹H NMR (500 MHz, CDCl₃) δ 8.81 (s, 1H), 7.85 (s, 1H), 7.53-7.43 (m, 1H), 7.40-7.27 (m, 3H), 6.58 (s, 1H), 6.17 (q, J=7.1 Hz, 1H), 3.45 (s, 3H), 2.29 (s, 3H), 2.13 (s, 3H), 2.06 (d, J=7.2 Hz, 3H); [M+H]⁺ 450.4.

### Example 46

### 7-(3,5-Dimethylisoxazol-4-yl)-8-methoxy-1-(p-tolylmethyl)oxazolo[5,4-c]quinolin-2-one

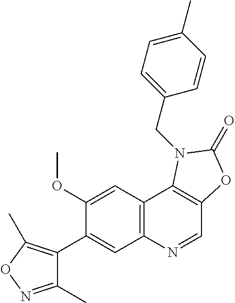 [SMILES: COc1cc2c(cc1-c1c(C)noc1C)ncc1oc(=O)n(Cc3ccc(C)cc3)c12]

A mixture of 7-(3,5-dimethylisoxazol-4-yl)-8-methoxy-1H-oxazolo[5,4-c]quinolin-2-one (see Example 11, 24.7 mg, 0.0800 mmol), Cs₂CO₃ (51.7 mg, 0.160 mmol), TBAI (29.3 mg, 0.0800 mmol), and 1-(bromomethyl)-4-methyl-benzene (29.4 mg, 0.160 mmol) in dry DMF (5.00 mL) was stirred at rt for 12 h. The mixture was concentrated under reduced pressure, and the residue was diluted with EtOAc (75.0 mL) and saturated aq NaHCO₃ (75.0 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) to provide the title compound as a solid (1.90 mg). ¹H NMR (500 MHz, CDCl₃) δ 8.80 (s, 1H), 7.86 (s, 1H), 7.24 (d, J=8.2 Hz, 2H), 7.20 (d, J=8.1 Hz, 2H), 7.05 (s, 1H), 5.49 (s, 2H), 3.60 (s, 3H), 2.34 (s, 3H), 2.31 (s, 3H), 2.15 (s, 3H); [M+H]⁺ 416.3.

### Example 47

### 1-Benzyl-4-(dimethylaminomethyl)-7-(3,5-dimethylisoxazol-4-yl)-8-methoxy-oxazolo[5,4-c]quinolin-2-one

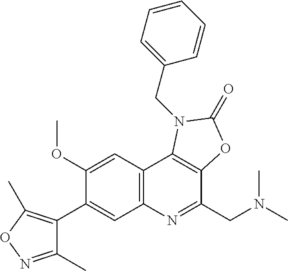 [SMILES: COc1cc2c(cc1-c1c(C)noc1C)nc(CN(C)C)c1oc(=O)n(Cc3ccccc3)c12]

A mixture of 1-benzyl-7-(3,5-dimethylisoxazol-4-yl)-8-methoxy-2-oxo-oxazolo[5,4-c]quinoline-4-carbaldehyde (see step 1 towards Example 33, 23.9 mg, 0.0560 mmol), 4 Å molecular sieves (200 mg), and dimethylamine (60 μL, 0.120 mmol) in DCE (4.00 mL) was stirred at rt for 2 h. NaBH(OAc)₃ (39.1 mg, 0.180 mmol) was added, and the mixture was stirred for 12 h. The mixture was diluted with saturated aq NaHCO₃ (25.0 mL) and DCM (25.0 mL). The aq phase was extracted with DCM (3×25.0 mL), and the combined organic phases were washed with brine, dried over MgSO₄, filtered, and concentrated under reduced pressure. The residue was diluted with HCl (0.500 mL, 1M in Et₂O), and the slurry was filtered. The solid was washed with Et₂O and dried under high vacuum to provide the hydrochloride salt of the title compound as a solid (24.6 mg, 96%). ¹H NMR (500 MHz, CDCl₃) δ 7.91 (s, 1H), 7.39-7.42 (m, 2H), 7.34-7.36 (m, 3H), 6.98 (s, 1H), 5.53 (s, 2H), 3.91 (s, 2H), 3.54 (s, 3H), 2.43 (s, 6H), 2.29 (s, 3H), 2.13 (s, 3H); [M+H]⁺ 459.5.

### Example 48

### N-[[1-Benzyl-7-(3,5-dimethylisoxazol-4-yl)-8-methoxy-2-oxo-oxazolo[5,4-c]quinolin-4-yl]methyl]-N-ethyl-acetamide

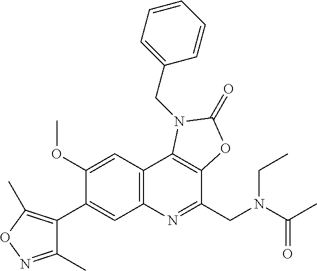 [SMILES: CCN(Cc1nc2cc(-c3c(C)noc3C)c(OC)cc2c2c1oc(=O)n2Cc1ccccc1)C(C)=O]

1-Benzyl-7-(3,5-dimethylisoxazol-4-yl)-4-(ethylamino methyl)-8-methoxy-oxazolo[5,4-c]quinolin-2-one (see Example 52, 19.5 mg, 0.0430 mmol) was dissolved in DCM (2.00 mL), and TEA (12.0 μL 0.0850 mmol) and acetyl chloride (6.00 μL 0.0860 mmol) were added. The mixture was stirred 4 h and then diluted with saturated aq NH₄Cl (10.0 mL). The aq phase was extracted with DCM (3×10 mL), and the combined organic phases were dried over Na₂SO₄, filtered, and concentrated under reduced pressure. The product was purified by HPLC (low pH) to provide the title compound as a solid (10.5 mg, 49%). ¹H NMR (300 MHz, MeOD) δ 8.10 (s, 1H), 7.41 (ddd, J=18.8, 13.7, 7.1 Hz, 5H), 7.30 (s, 1H), 5.74 (s, 2H), 5.07 (s, 2H), 3.79 (q, J=6.9 Hz, 2H), 3.64 (s, 3H), 2.30 (s, 3H), 2.21 (s, 3H), 2.11 (s, 3H), 1.37 (t, J=6.8 Hz, 4H); [M+H]⁺ 488.3.

### Example 49

### 7-(3,5-Dimethylisoxazol-4-yl)-8-methoxy-1-[(2-methylsulfonylphenyl)methyl]oxazolo[5,4-c]quinolin-2-one

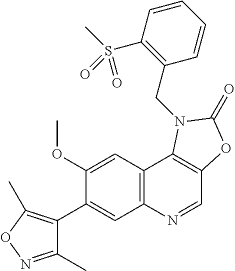 [SMILES: COc1cc2c(cc1-c1c(C)noc1C)ncc1oc(=O)n(Cc3ccccc3S(C)(=O)=O)c12]

A mixture of 7-(3,5-dimethylisoxazol-4-yl)-8-methoxy-1H-oxazolo[5,4-c]quinolin-2-one (see Example 11, 27.5 mg, 0.0900 mmol), Cs₂CO₃ (57.6 mg, 0.180 mmol), TBAI (32.6 mg, 0.0900 mmol), and 1-(bromomethyl)-2-methylsulfonyl-benzene (44.0 mg, 0.180 mmol) in dry DMF (5.00 mL) was stirred at rt for 48 h. The mixture was concentrated under reduced pressure, and the residue was diluted with EtOAc (75.0 mL) and saturated aq NaHCO₃ (75.0 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) followed by flash chromatography on silica gel, eluting with mixtures of hexanes and EtOAc to provide the title compound as a solid (4.06 mg). ¹H NMR (500 MHz, CDCl₃) δ 8.84 (s, 1H), 8.22 (dd, J=5.4, 3.9 Hz, 1H), 7.88 (s, 1H), 7.70-7.51 (m, 2H), 7.24-7.13 (m, 1H), 6.91 (s, 1H), 6.14 (s, 2H), 3.57 (s, 3H), 3.27 (s, 3H), 2.30 (s, 3H), 2.13 (s, 3H); [M+H]⁺ 480.3.

### Example 50

### 2-[[7-(3,5-Dimethylisoxazol-4-yl)-8-methoxy-2-oxo-oxazolo[5,4-c]quinolin-1-yl]methyl]benzonitrile

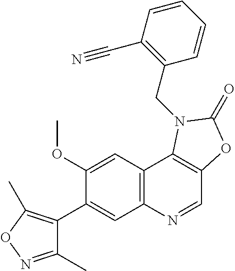 [SMILES: COc1cc2c(cc1-c1c(C)noc1C)ncc1oc(=O)n(Cc3ccccc3C#N)c12]

A mixture of 7-(3,5-dimethylisoxazol-4-yl)-8-methoxy-1H-oxazolo[5,4-c]quinolin-2-one (see Example 11, 20.0 mg, 0.0600 mmol), Cs₂CO₃ (41.9 mg, 0.130 mmol), and 2-(bromomethyl)benzonitrile (25.2 mg, 0.130 mmol) in dry MeCN (5.00 mL) was stirred at rt for 16 h. The mixture was concentrated under reduced pressure, and the residue was diluted with EtOAc (75.0 mL) and saturated aq NaHCO₃ (75.0 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) to provide the title compound as a solid (4.38 mg). ¹H NMR (500 MHz, CDCl₃) δ 8.84 (s, 1H), 7.89 (s, 1H), 7.84 (dd, J=7.7, 1.1 Hz, 1H), 7.61 (td, J=7.8, 1.3 Hz, 1H), 7.51 (dd, J=7.6, 6.8 Hz, 1H), 7.28-7.23 (m, 1H), 6.79 (s, 1H), 5.78 (s, 2H), 3.64 (s, 3H), 2.30 (s, 3H), 2.14 (s, 3H); [M+H]⁺ 427.3.

### Example 51

### 7-(3,5-Dimethylisoxazol-4-yl)-8-methoxy-2-oxo-1-[(1R)-1-phenylethyl]oxazolo[5,4-c]quinoline-4-carboxylic acid

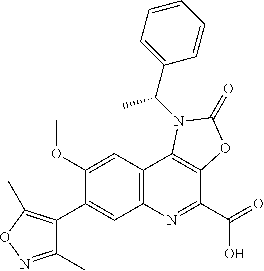 [SMILES: COc1cc2c(cc1-c1c(C)noc1C)nc(C(=O)O)c1oc(=O)n([C@H](C)c3ccccc3)c12]

A mixture of ethyl 7-(3,5-dimethylisoxazol-4-yl)-8-methoxy-2-oxo-1-[(1R)-1-phenylethyl]oxazolo[5,4-c]quinoline-4-carboxylate (see Example 34, 43.9 mg, 0.0900 mmol) in HCl (11.3 mL, 67.5 mmol, 6M aq) was heated to 80° C. for 6 h. The mixture was cooled to rt and concentrated under reduced pressure. The residue was diluted with water (75.0 mL), and the pH was adjusted to 7 with saturated aq NaHCO₃. EtOAc (75.0 mL) was added, and the aq phase was extracted with EtOAc (3×75.0 mL). The combined organic phases were washed with brine (100 mL), dried over MgSO₄, filtered, and concentrated under reduced pressure. The product was purified by HPLC (high pH) to provide the title compound as a solid (6.58 mg, 16%). ¹H NMR (500 MHz, CDCl₃) δ 7.87 (s, 1H), 7.55-7.33 (m, 5H), 6.67 (s, 1H), 6.29 (dd, J=14.4, 7.1 Hz, 1H), 3.37 (s, 3H), 2.31 (s, 3H), 2.14 (s, 3H), 2.08 (d, J=7.2 Hz, 3H); [M+H]⁺ 460.4.

### Example 52

### 1-Benzyl-7-(3,5-dimethylisoxazol-4-yl)-4-(ethylaminomethyl)-8-methoxy-oxazolo[5,4-c]quinolin-2-one

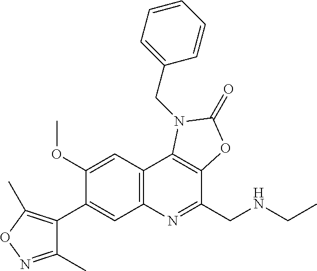 [SMILES: CCNCc1nc2cc(-c3c(C)noc3C)c(OC)cc2c2c1oc(=O)n2Cc1ccccc1]

NaBH(OAc)₃ (16.0 mg, 0.073 mmol) was added to a mixture of 1-benzyl-7-(3,5-dimethylisoxazol-4-yl)-8-methoxy-2-oxo-oxazolo[5,4-c]quinoline-4-carbaldehyde (see Intermediate step 1, Example 33, 21.0 mg, 0.0500 mmol) and ethanamine (0.03 mL, 0.059 mmol, 2.00 M in THF) in DCE (2.00 mL). The mixture was stirred at room temperature for 30 m, and saturated aq NaHCO₃ (5.00 mL). The aq phase was extracted with DCM (3×10 mL), and the combined organic phases were dried over Na₂SO₄ and concentrated under pressure. The product was purified by HPLC (low pH) to provide the title compound as a solid (22.0 mg, 98%). ¹H NMR (300 MHz, CDCl₃) δ 7.83 (s, 1H), 7.44-7.30 (m, 6H), 6.96 (s, 1H), 5.52 (s, 2H), 4.23 (s, 2H), 3.54 (s, 3H), 2.82 (q, J=7.1 Hz, 2H), 2.29 (s, 3H), 2.13 (s, 3H), 2.02 (br, 1H), 1.20 (t, J=7.1 Hz, 3H); [M+H]⁺ 459.2.

### Example 53

### 7-(3,5-Dimethylisoxazol-4-yl)-8-methoxy-2-[(3-methylsulfonylphenyl)methoxy]oxazolo[5,4-c]quinoline

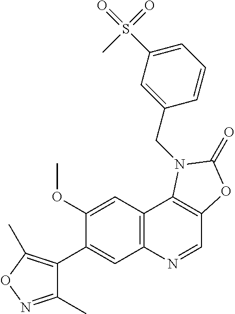 [SMILES: COc1cc2c(cc1-c1c(C)noc1C)ncc1oc(=O)n(Cc3cccc(S(C)(=O)=O)c3)c12]

A mixture of 7-(3,5-dimethylisoxazol-4-yl)-8-methoxy-1H-oxazolo[5,4-c]quinolin-2-one (see Example 11, 27.5 mg, 0.0900 mmol), Cs₂CO₃ (57.6 mg, 0.180 mmol), TBAI (32.6 mg, 0.0900 mmol), and 1-(bromomethyl)-3-methylsulfonyl-benzene (44.0 mg, 0.180 mmol) in dry DMF (5.00 mL) was stirred at rt for 48 h. The mixture was concentrated under reduced pressure, and the residue was diluted with EtOAc (75.0 mL) and saturated aq NaHCO₃ (75.0 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) followed by flash chromatography on silica gel, eluting with mixtures of DCM and MeOH to provide the title compound as a solid (3.78 mg). ¹H NMR (500 MHz, CDCl₃) δ 8.01 (s, 1H), 7.98-7.92 (m, 2H), 7.83 (s, 1H), 7.62 (t, J=7.8 Hz, 1H), 7.34 (d, J=7.9 Hz, 1H), 7.28 (s, 1H), 5.76 (s, 2H), 3.96 (s, 3H), 3.05 (s, 3H), 2.05 (s, 3H), 1.96 (s, 3H); [M+H]⁺ 480.3.

### Example 54

### 7-(3,5-Dimethylisoxazol-4-yl)-8-methoxy-1-[[4-(trifluoromethoxy)phenyl]methyl]oxazolo[5,4-c]quinolin-2-one

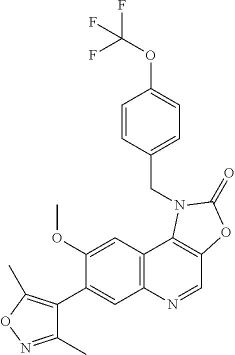 [SMILES: COc1cc2c(cc1-c1c(C)noc1C)ncc1oc(=O)n(Cc3ccc(OC(F)(F)F)cc3)c12]

A mixture of 7-(3,5-dimethylisoxazol-4-yl)-8-methoxy-1H-oxazolo[5,4-c]quinolin-2-one (see Example 11, 29.1 mg, 0.0935 mmol), Cs₂CO₃ (60.9 mg, 0.186 mmol), TBAI (34.5 mg, 0.094 mmol), and 4-(trifluoromethoxy)benzyl bromide (47.7 mg, 186.9 mmol) in dry DMF (5.00 mL) was stirred at rt for 17 h. The mixture was concentrated under reduced pressure, and the residue was diluted with EtOAc (25.0 mL) and saturated aq NaHCO₃ (25.0 mL). The aq phase was extracted with EtOAc (3×25.0 mL), and the combined organic phases were washed with brine (30 mL), dried over Na₂SO₄, filtered, and concentrated under reduced pressure. The product was purified by flash chromatography, eluting with mixtures of hexanes and EtOAc to provide the title product as a solid (16.6 mg, 36%). ¹H NMR (500 MHz, CDCl₃) δ 8.82 (s, 1H), 7.90 (s, 1H), 7.41 (d, J=8.8 Hz, 2H), 7.28 (d, J=8.1 Hz, 2H), 6.93 (s, 1H), 5.54 (s, 2H), 3.57 (s, 3H), 2.31 (s, 3H), 2.15 (s, 3H); [M+H]⁺ 486.5.

### Example 55

### 7-(3,5-Dimethylisoxazol-4-yl)-8-methoxy-1-[1-(4-pyridyl)ethyl]oxazolo[5,4-c]quinolin-2-one

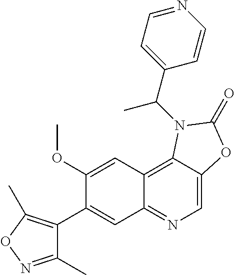 [SMILES: COc1cc2c(cc1-c1c(C)noc1C)ncc1oc(=O)n(C(C)c3ccncc3)c12]

A mixture of 1-(4-pyridyl)ethanone (1.06 g, 8.75 mmol) in MeOH (50.0 mL) was cooled to 0° C., and NaBH₄ (331 mg, 8.75 mmol) was added in portions. The mixture was stirred at 0° C. for 30 m, and acetone (25.0 mL) was added in drops. Saturated aq Na/K tartrate (5.00 mL) was added, and the mixture was warmed to rt and concentrated under reduced pressure. The residue was diluted with water (25.0 mL) and EtOAc (75.0 mL). The aq phase was extracted with EtOAc (6×25.0 mL), and the combined organic phases were washed with brine (100 mL), dried over MgSO₄, filtered, and concentrated under reduced pressure to provide 1-(4-pyridyl)ethanol as a solid. ¹H NMR (500 MHz, CDCl3) δ 8.43 (dd, J=4.5, 1.6 Hz, 2H), 7.38-7.18 (m, 2H), 4.87 (q, J=6.6 Hz, 1H), 3.92 (s, 1H), 1.47 (d, J=6.6 Hz, 3H). A mixture of PPh₃ (70.0 mg, 0.270 mmol) in dry THF (3.00 mL) was cooled to 0° C., and DIAD (52.1 uL, 0.260 mmol) was added. A mixture of 7-(3,5-dimethylisoxazol-4-yl)-8-methoxy-1H-oxazolo[5,4-c]quinolin-2-one (see Example 11, 32.0 mg, 0.100 mmol) in dry THF (3.00 mL) was added. The mixture was stirred at 0° C. for 10 m, and a solution of 1-(4-pyridyl)ethanol (36.7 mg, 0.300 mmol) in dry THF (3.00 mL) was added. The mixture was warmed to rt and stirred for 4 d. The mixture was diluted with EtOAc (100 mL) and saturated aq NaHCO₃ aq (100 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) followed by flash chromatography on silica gel, eluting with mixtures of EtOAc and MeOH to provide the title compound as a solid (2.8 mg, 6.5%). ¹H NMR (500 MHz, CDCl₃) δ 8.85 (s, 1H), 8.69 (m, 2H), 7.88 (s, 1H), 7.37 (d, J=4.3 Hz, 2H), 6.40 (s, 1H), 6.20 (q, J=7.2 Hz, 1H), 3.35 (s, 3H), 2.30 (s, 3H), 2.13 (s, 3H), 2.09 (d, J=7.2 Hz, 3H); [M+H]⁺ 417.3.

### Example 56

### 7-(3,5-Dimethylisoxazol-4-yl)-8-methoxy-1-[1-(3-pyridyl)ethyl]oxazolo[5,4-c]quinolin-2-one

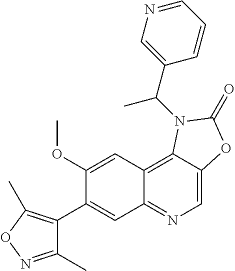 [SMILES: COc1cc2c(cc1-c1c(C)noc1C)ncc1oc(=O)n(C(C)c3cccnc3)c12]

A mixture of 1-(3-pyridyl)ethanone (1.04 g, 8.59 mmol) in MeOH (50.0 mL) was cooled to 0° C., and NaBH₄ (325 mg, 8.59 mmol) was added in portions. The mixture was stirred at 0° C. for 30 m, and acetone (25.0 mL) was added in drops. Saturated aq Na/K tartrate (5.00 mL) was added, and the mixture was slowly warmed to rt and concentrated under reduced pressure. The residue was diluted with water (25.0 mL) and EtOAc (75.0 mL). The aq phase was extracted with EtOAc (6×25.0 mL), and the combined organic phases were washed with brine (100 mL), dried over MgSO₄, filtered, and concentrated under reduced pressure to provide 1-(3-pyridyl)ethanol as a solid. ¹H NMR (500 MHz, CDCl₃) δ 8.39 (d, J=41.8 Hz, 2H), 7.89-7.57 (m, 1H), 7.27-7.13 (m, 1H), 4.88 (dd, J=5.6, 3.5 Hz, 1H), 4.33 (s, 1H), 1.64-1.34 (m, 3H). A mixture of PPh₃ (68.8 mg, 0.260 mmol) in dry THF (5.00 mL) was cooled to 0° C., and DIAD (51.2 μL, 0.260 mmol) was added. A mixture of 7-(3,5-dimethylisoxazol-4-yl)-8-methoxy-1H-oxazolo[5,4-c]quinolin-2-one (see Example 11, 31.4 mg, 0.100 mmol) in dry THF (5.00 mL) was added. The mixture was stirred at 0° C. for 10 m, and a solution of 1-(3-pyridyl)ethanol (36.0 mg, 0.290 mmol) in dry THF (5.00) was added. The mixture was warmed to rt and stirred for 3 d. The mixture was diluted with EtOAc (100 mL) and saturated aq NaHCO₃ (100 mL). The aq phase was extracted with EtOAc (3×75.0 mL), and the combined organic phases were washed with brine (100 mL), dried over MgSO₄, filtered, and concentrated under reduced pressure. The product was purified by HPLC (high pH) followed by flash chromatography on silica gel, eluting with mixtures of EtOAc and MeOH to provide the title compound as a solid (2.0 mg, 4.7%). ¹H NMR (500 MHz, CDCl₃) δ 8.84 (s, 1H), 8.82 (m, 1H), 8.66 (m, 1H), 7.90 (s, 1H), 7.74 (d, J=5.9 Hz, 1H), 7.41-7.33 (m, 1H), 6.58 (s, 1H), 6.28 (q, J=7.2 Hz, 1H), 3.44 (s, 3H), 2.30 (s, 3H), 2.15 (d, J=5.6 Hz, 3H), 2.14 (s, 3H); [M+H]⁺ 417.4.

### Intermediate Step 1 Towards Example 57

### 7-(3,5-Dimethylisoxazol-4-yl)-4-(hydroxymethyl)-8-methoxy-1H-oxazolo[5,4-c]quinolin-2-one

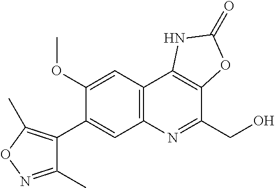 [SMILES: COc1cc2c(cc1-c1c(C)noc1C)nc(CO)c1oc(=O)[nH]c12]

MsOH (7.3 μL, 0.112 mmol) and anisole (12.2 μL, 0.112 mmol) were added to a solution of 7-(3,5-dimethylisoxazol-4-yl)-4-(hydroxymethyl)-8-methoxy-1-[(1R)-1-phenylethyl]oxazolo[5,4-c]quinolin-2-one (see Example 35, 25.0 mg, 0.0602 mmol) in toluene (5.00 mL). The mixture was stirred at 80° C. for 16 h. The mixture was cooled to rt, and saturated aq NaHCO₃ (75.0 mL) was added. The aq phase was extracted with EtOAc (3×20.0 mL), and the combined organic phases were dried over Na₂SO₄, filtered, and concentrated under reduced pressure. The product was purified by flash chromatography, eluting with mixtures of EtOAc and hexanes to provide the title compound as a solid (12.5 mg, 65%). ¹H NMR (500 MHz, MeOD) δ 7.90 (s, 1H), 7.56 (s, 1H), 4.98 (s, 2H), 4.00 (s, 3H), 2.36 (s, 3H), 2.19 (s, 3H); [M+H]⁺ 342.4.

### Example 57

### 2-[[7-(3,5-Dimethylisoxazol-4-yl)-4-(hydroxymethyl)-8-methoxy-2-oxo-oxazolo[5,4-c]quinolin-1-yl]methyl]benzonitrile

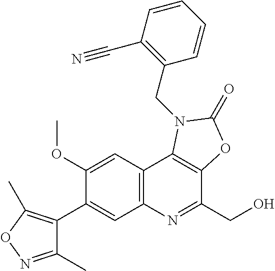 [SMILES: COc1cc2c(cc1-c1c(C)noc1C)nc(CO)c1oc(=O)n(Cc3ccccc3C#N)c12]

A mixture of 7-(3,5-dimethylisoxazol-4-yl)-4-(hydroxymethyl)-8-methoxy-1H-oxazolo[5,4-c]quinolin-2-one (12.5 mg, 36.6 mmol), Cs₂CO₃ (23.9 mg, 73.2 mmol), TBAI (13.5 mg, 36.6 mmol), and 1-(bromomethyl)-2-cyano-benzene (14.4 mg, 73.2 mmol) in dry DMF (2.00 mL) was stirred at rt for 12 h. The mixture was concentrated under reduced pressure, and the residue was diluted with EtOAc (25.0 mL) and saturated aq NaHCO₃ (25.0 mL). The aq phase was extracted with EtOAc (3×25.0 mL), and the combined organic phases were washed with brine (30 mL), dried over NaSO₄, filtered, and concentrated under reduced pressure. The product was purified by HPLC (high pH) followed by flash chromatography on silica gel, eluting with mixtures of EtOAc and hexanes to provide the title compound as a solid (1.9 mg, 11%). ¹H NMR (500 MHz, CDCl₃) δ 8.34 (s, 1H), 7.86 (d, J=7.4 Hz, 1H), 7.67 (s, 1H), 7.56 (s, 1H), 7.30 (s, 1H), 6.89 (s, 1H), 5.84 (s, 2H), 5.25 (s, 2H), 3.70 (s, 3H), 2.33 (s, 3H), 2.15 (s, 3H); [M+H]⁺ 457.4.

TABLE 2
Examples 58-61 can be prepared according to the procedure outlined in Example 55 for
7-(3,5-dimethylisoxazol-4-yl)-8-methoxy-1-[(1R)-1-(4-pyridyl)ethyl]oxazolo[5,4-c]
quinolin-2-one, by replacing 1-(4-pyridyl)ethanone with the appropriate ketone.

| Example | Structure | Name |
|---|---|---|
| 58 | 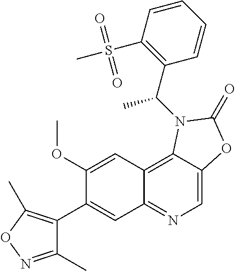 [SMILES: COc1cc2c(cc1-c1c(C)noc1C)ncc1oc(=O)n([C@H](C)c3ccccc3S(C)(=O)=O)c12] | 7-(3,5-Dimethylisoxazol-4-yl)-8- methoxy-1-[(1R)-1-(2-methylsulfonyl- phenyl)ethyl]oxazolo[5,4-c]quinolin- 2-one |
| 59 | 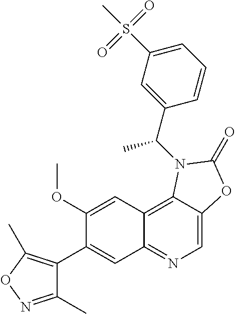 [SMILES: COc1cc2c(cc1-c1c(C)noc1C)ncc1oc(=O)n([C@H](C)c3cccc(S(C)(=O)=O)c3)c12] | 7-(3,5-Dimethylisoxazol-4-yl)-8- methoxy-1-[(1R)-1-(3-methyl- sulfonylphenyl)ethyl]oxazolo[5,4- c]quinolin-2-one |
| 60 | 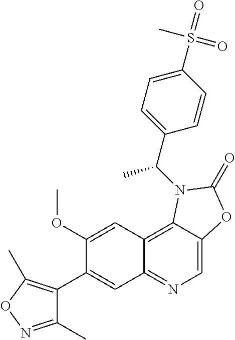 [SMILES: COc1cc2c(cc1-c1c(C)noc1C)ncc1oc(=O)n([C@H](C)c3ccc(S(C)(=O)=O)cc3)c12] | 7-(3,5-Dimethylisoxazol-4-yl)-8- methoxy-1-[(1R)-1-(4-methyl- sulfonylphenyl)ethyl]oxazolo[5,4- c]quinolin-2-one |
| 61 | 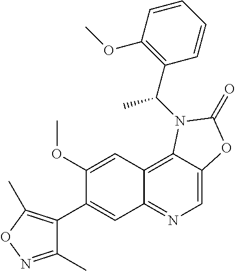 [SMILES: COc1cc2c(cc1-c1c(C)noc1C)ncc1oc(=O)n([C@H](C)c3ccccc3OC)c12] | 7-(3,5-Dimethylisoxazol-4-yl)-8- methoxy-1-[(1R)-1-(2-methoxy- phenyl)ethyl]oxazolo[5,4- c]quinolin-2-one |

TABLE 3
Examples 62-65 can be prepared according to the procedure outlined in Example 45 for
1-[(1R)-1-(3-chlorophenyl)ethyl]-7-(3,5-dimethylisoxazol-4-yl)-8-methoxy-oxazolo[5,4-c]
quinolin-2-one, by replacing (1R)-1-(3-chlorophenyl)ethanamine with the appropriate amine.

| Example | Structure | Name |
|---|---|---|
| 62 | 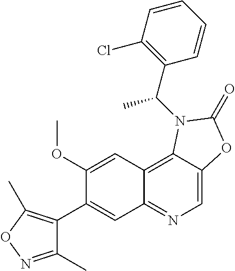 [SMILES: COc1cc2c(cc1-c1c(C)noc1C)ncc1oc(=O)n([C@H](C)c3ccccc3Cl)c12] | 1-[(1R)-1-(2-Chlorophenyl)ethyl]-7- (3,5-dimethylisoxazol-4-yl)-8- methoxy-oxazolo[5,4-c]quinolin-2- one |
| 63 | 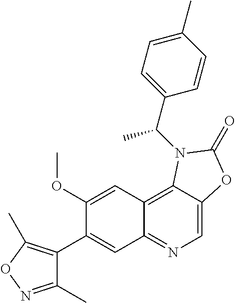 [SMILES: COc1cc2c(cc1-c1c(C)noc1C)ncc1oc(=O)n([C@H](C)c3ccc(C)cc3)c12] | 7-(3,5-Dimethylisoxazol-4-yl)-8- methoxy-1-[(1R)-1-(p- tolyl)ethyl]oxazolo[5,4-c]quinolin- 2-one |
| 64 | 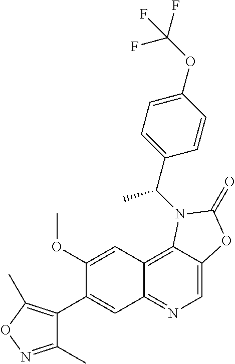 [SMILES: COc1cc2c(cc1-c1c(C)noc1C)ncc1oc(=O)n([C@H](C)c3ccc(OC(F)(F)F)cc3)c12] | 7-(3,5-Dimethylisoxazol-4-yl)-8- methoxy-1-[(1R)-1-[4-(trifluoro- methoxy)phenyl]ethyl]oxazolo[5,4- c]quinolin-2-one |
| 65 | 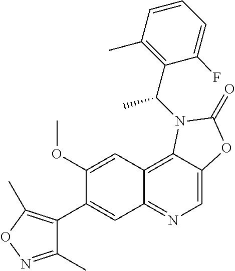 [SMILES: COc1cc2c(cc1-c1c(C)noc1C)ncc1oc(=O)n([C@H](C)c3c(C)cccc3F)c12] | 7-(3,5-Dimethylisoxazol-4-yl)-1- [(1R)-1-(2-fluoro-6-methyl- phenyl)ethyl]-8-methoxy- oxazolo[5,4-c]quinolin-2-one |

TABLE 4
Examples 66-78 can be prepared according to the procedure outlined in steps 1 and 2,
Example 12A for 3-[(1R)-1-phenylethyl]oxazol-2-one, by replacing (1R)-1-phenylethanamine
with the appropriate amine, followed by the procedure outlined in Example 35 for
7-(3,5-dimethyl-isoxazol-4-yl)-4-(hydroxymethyl)-8-methoxy-1-[(1R)-1-phenylethyl]
oxazolo[5,4-c]quinolin-2-one, replacing 3-[(1R)-1-phenylethyl]oxazol-2-one with the
appropriate oxazol-2-one-dienophile.

| Example | Structure | Name |
|---|---|---|
| 66 | 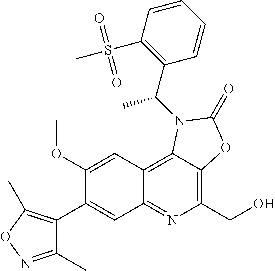 [SMILES: COc1cc2c(cc1-c1c(C)noc1C)nc(CO)c1oc(=O)n([C@H](C)c3ccccc3S(C)(=O)=O)c12] | 7-(3,5-Dimethylisoxazol-4-yl)-4- (hydroxymethyl)-8-methoxy-1- [(1R)-1-(2-methylsulfonyl- phenyl)ethyl]oxazolo[5,4- c]quinolin-2-one |
| 67 | 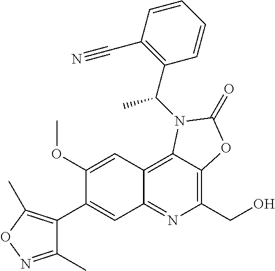 [SMILES: COc1cc2c(cc1-c1c(C)noc1C)nc(CO)c1oc(=O)n([C@H](C)c3ccccc3C#N)c12] | 2-[(1R)-1-[7-(3,5-Dimethylisoxazol- 4-yl)-4-(hydroxymethyl)-8- methoxy-2-oxo-oxazolo[5,4- c]quinolin-1-yl]ethyl]benzonitrile |
| 68 | 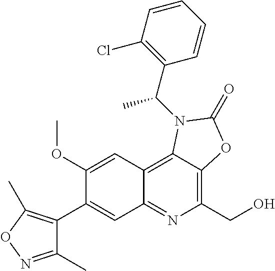 [SMILES: COc1cc2c(cc1-c1c(C)noc1C)nc(CO)c1oc(=O)n([C@H](C)c3ccccc3Cl)c12] | 1-[(1R)-1-(2-Chlorophenyl)ethyl]-7- (3,5-dimethylisoxazol-4-yl)-4- (hydroxymethyl)-8-methoxy- oxazolo[5,4-c]quinolin-2-one |
| 69 | 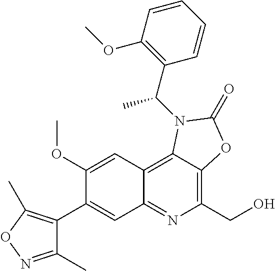 [SMILES: COc1cc2c(cc1-c1c(C)noc1C)nc(CO)c1oc(=O)n([C@H](C)c3ccccc3OC)c12] | 7-(3,5-Dimethylisoxazol-4-yl)-4- (hydroxymethyl)-8-methoxy-1- [(1R)-1-(2-methoxy- phenyl)ethyl]oxazolo[5,4- c]quinolin-2-one |
| 70 | 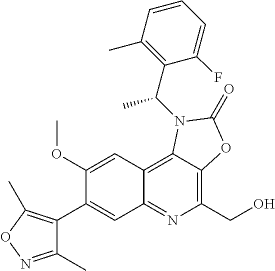 [SMILES: COc1cc2c(cc1-c1c(C)noc1C)nc(CO)c1oc(=O)n([C@H](C)c3c(C)cccc3F)c12] | 7-(3,5-Dimethylisoxazol-4-yl)-1- [(1R)-1-(2-fluoro-6-methyl- phenyl)ethyl]-4-(hydroxymethyl)-8- methoxy-oxazolo[5,4-c]quinolin-2- one |
| 71 | 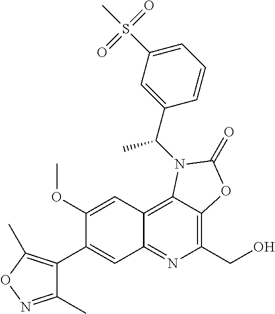 [SMILES: COc1cc2c(cc1-c1c(C)noc1C)nc(CO)c1oc(=O)n([C@H](C)c3cccc(S(C)(=O)=O)c3)c12] | 7-(3,5-Dimethylisoxazol-4-yl)-4- (hydroxymethyl)-8-methoxy-1- [(1R)-1-(3-methylsulfonyl- phenyl)ethyl]oxazolo[5,4- c]quinolin-2-one |
| 72 | 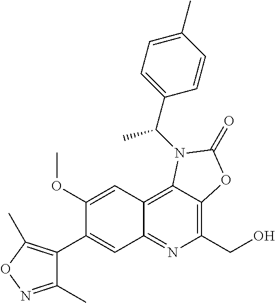 [SMILES: COc1cc2c(cc1-c1c(C)noc1C)nc(CO)c1oc(=O)n([C@H](C)c3ccc(C)cc3)c12] | 7-(3,5-Dimethylisoxazol-4-yl)-4- (hydroxymethyl)-8-methoxy-1- [(1R)-1-(p-tolyl)ethyl]oxazolo[5,4- c]quinolin-2-one |
| 73 | 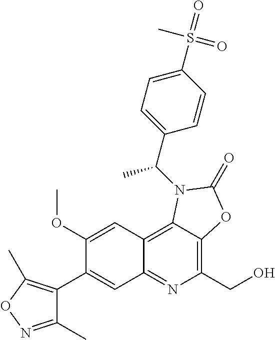 [SMILES: COc1cc2c(cc1-c1c(C)noc1C)nc(CO)c1oc(=O)n([C@H](C)c3ccc(S(C)(=O)=O)cc3)c12] | 7-(3,5-Dimethylisoxazol-4-yl)-4- (hydroxymethyl)-8-methoxy-1- [(1R)-1-(4-methylsulfonyl- phenyl)ethyl]oxazolo[5,4- c]quinolin-2-one |
| 74 | 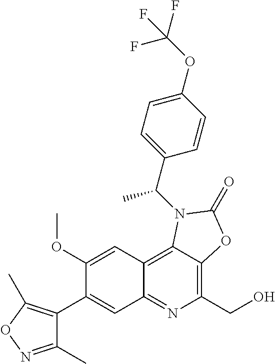 [SMILES: COc1cc2c(cc1-c1c(C)noc1C)nc(CO)c1oc(=O)n([C@H](C)c3ccc(OC(F)(F)F)cc3)c12] | 7-(3,5-Dimethylisoxazol-4-yl)-4- (hydroxymethyl)-8-methoxy-1- [(1R)-1-[4-(trifluoro- methoxy)phenyl]ethyl]oxazolo[5,4- c]quinolin-2-one |
| 75 | 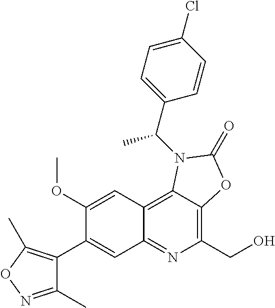 [SMILES: COc1cc2c(cc1-c1c(C)noc1C)nc(CO)c1oc(=O)n([C@H](C)c3ccc(Cl)cc3)c12] | 1-[(1R)-1-(4-Chlorophenyl)ethyl]-7- (3,5-dimethylisoxazol-4-yl)-4- (hydroxymethyl)-8-methoxy- oxazolo[5,4-c]quinolin-2-one |
| 76 | 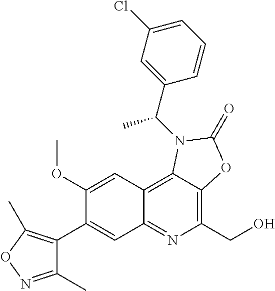 [SMILES: COc1cc2c(cc1-c1c(C)noc1C)nc(CO)c1oc(=O)n([C@H](C)c3cccc(Cl)c3)c12] | 1-[(1R)-1-(3-Chlorophenyl)ethyl]-7- (3,5-dimethylisoxazol-4-yl)-4- (hydroxymethyl)-8-methoxy- oxazolo[5,4-c]quinolin-2-one |
| 77 | 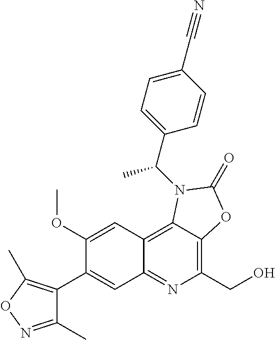 [SMILES: COc1cc2c(cc1-c1c(C)noc1C)nc(CO)c1oc(=O)n([C@H](C)c3ccc(C#N)cc3)c12] | 4-[(1R)-1-[7-(3,5-Dimethylisoxazol- 4-yl)-4-(hydroxymethyl)-8- methoxy-2-oxo-oxazolo[5,4- c]quinolin-1-yl]ethyl]benzonitrile |
| 78 | 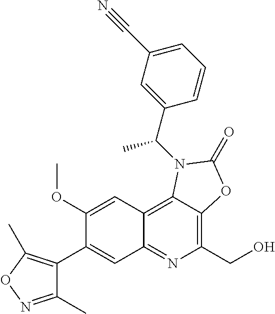 [SMILES: COc1cc2c(cc1-c1c(C)noc1C)nc(CO)c1oc(=O)n([C@H](C)c3cccc(C#N)c3)c12] | 3-[(1R)-1-[7-(3,5-Dimethylisoxazol- 4-yl)-4-(hydroxymethyl)-8- methoxy-2-oxo-oxazolo[5,4- c]quinolin-1-yl]ethyl]benzonitrile |

TABLE 5
Examples 79-90 can be prepared according to the procedure outlined in Example 57 for 2-[[7-
(3,5-dimethylisoxazol-4-yl)-4-(hydroxymethyl)-8-methoxy-2-oxo-oxazolo[5,4-c]quinolin-1-yl]
methyl]benzonitrile, by replacing 1-(bromomethyl)-2-cyano-benzene with the appropriate halide.

| Example | Structure | Name |
|---|---|---|
| 79 | 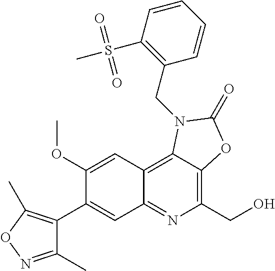 [SMILES: COc1cc2c(cc1-c1c(C)noc1C)nc(CO)c1oc(=O)n(Cc3ccccc3S(C)(=O)=O)c12] | 7-(3,5-Dimethylisoxazol-4-yl)-4- (hydroxymethyl)-8-methoxy-1-[(2- methylsulfonylphenyl)methyl] oxazolo[5,4-c]quinolin-2-one |
| 80 | 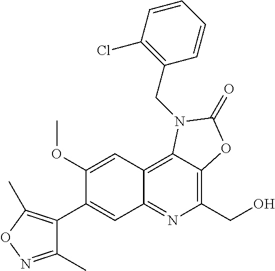 [SMILES: COc1cc2c(cc1-c1c(C)noc1C)nc(CO)c1oc(=O)n(Cc3ccccc3Cl)c12] | 1-[(2-Chlorophenyl)methyl]-7-(3,5- dimethylisoxazol-4-yl)-4- (hydroxymethyl)-8-methoxy- oxazolo[5,4-c]quinolin-2-one |
| 81 | 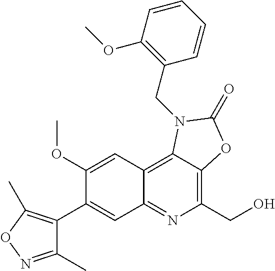 [SMILES: COc1ccccc1Cn1c(=O)oc2c(CO)nc3cc(-c4c(C)noc4C)c(OC)cc3c21] | 7-(3,5-Dimethylisoxazol-4-yl)-4- (hydroxymethyl)-8-methoxy-1-[(2- methoxyphenyl)methyl]oxazolo[5,4- c]quinolin-2-one |
| 82 | 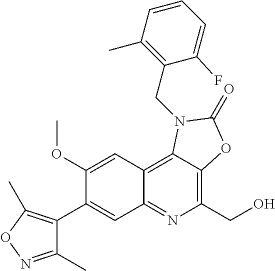 [SMILES: COc1cc2c(cc1-c1c(C)noc1C)nc(CO)c1oc(=O)n(Cc3c(C)cccc3F)c12] | 7-(3,5-Dimethylisoxazol-4-yl)-1-[(2- fluoro-6-methyl-phenyl)methyl]-4- (hydroxymethyl)-8-methoxy- oxazolo[5,4-c]quinolin-2-one |
| 83 | 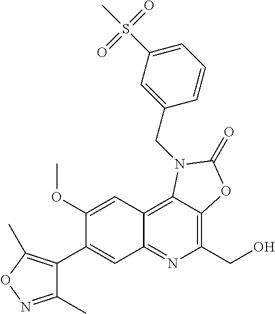 [SMILES: COc1cc2c(cc1-c1c(C)noc1C)nc(CO)c1oc(=O)n(Cc3cccc(S(C)(=O)=O)c3)c12] | 7-(3,5-Dimethylisoxazol-4-yl)-4- (hydroxymethyl)-8-methoxy-1-[(3- methylsulfonylphenyl)methyl] oxazolo[5,4-c]quinolin-2-one |
| 84 | 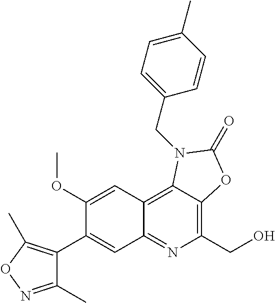 [SMILES: COc1cc2c(cc1-c1c(C)noc1C)nc(CO)c1oc(=O)n(Cc3ccc(C)cc3)c12] | 7-(3,5-Dimethylisoxazol-4-yl)-4- (hydroxymethyl)-8-methoxy-1-(p- tolylmethyl)oxazolo[5,4-c]quinolin- 2-one |
| 85 | 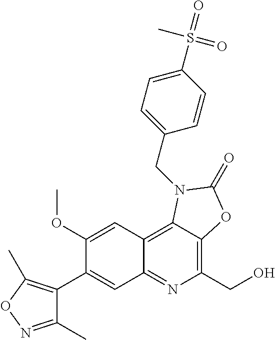 [SMILES: COc1cc2c(cc1-c1c(C)noc1C)nc(CO)c1oc(=O)n(Cc3ccc(S(C)(=O)=O)cc3)c12] | 7-(3,5-Dimethylisoxazol-4-yl)-4- (hydroxymethyl)-8-methoxy-1-[(4- methylsulfonylphenyl)methyl] oxazolo[5,4-c]quinolin-2-one |
| 86 | 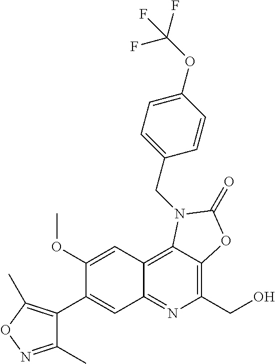 [SMILES: COc1cc2c(cc1-c1c(C)noc1C)nc(CO)c1oc(=O)n(Cc3ccc(OC(F)(F)F)cc3)c12] | 7-(3,5-Dimethylisoxazol-4-yl)-4- (hydroxymethyl)-8-methoxy-1-[[4- (trifluoromethoxy)phenyl]methyl] oxazolo[5,4-c]quinolin-2-one |
| 87 | 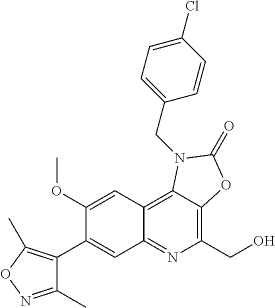 [SMILES: COc1cc2c(cc1-c1c(C)noc1C)nc(CO)c1oc(=O)n(Cc3ccc(Cl)cc3)c12] | 1-[(4-Chlorophenyl)methyl]-7-(3,5- dimethylisoxazol-4-yl)-4- (hydroxymethyl)-8-methoxy- oxazolo[5,4-c]quinolin-2-one |
| 88 | 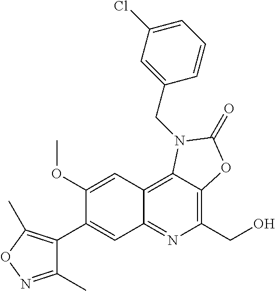 [SMILES: COc1cc2c(cc1-c1c(C)noc1C)nc(CO)c1oc(=O)n(Cc3cccc(Cl)c3)c12] | 1-[(3-Chlorophenyl)methyl]-7-(3,5- dimethylisoxazol-4-yl)-4- (hydroxymethyl)-8-methoxy- oxazolo[5,4-c]quinolin-2-one |
| 89 | 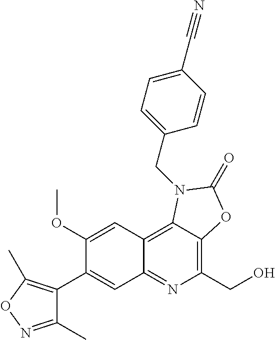 [SMILES: COc1cc2c(cc1-c1c(C)noc1C)nc(CO)c1oc(=O)n(Cc3ccc(C#N)cc3)c12] | 4-[[7-(3,5-Dimethylisoxazol-4-yl)-4- (hydroxymethyl)-8-methoxy-2-oxo- oxazolo[5,4-c]quinolin-1- yl]methyl]benzonitrile |
| 90 | 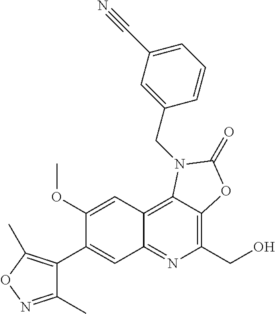 [SMILES: COc1cc2c(cc1-c1c(C)noc1C)nc(CO)c1oc(=O)n(Cc3cccc(C#N)c3)c12] | 3-[[7-(3,5-Dimethylisoxazol-4-yl)-4- (hydroxymethyl)-8-methoxy-2-oxo- oxazolo[5,4-c]quinolin-1- yl]methyl]benzonitrile |

TABLE 6
Example 91 can be prepared according to the procedure outlined in
Example 43 for 4-[(1R)-1-[7-(3,5-dimethylisoxazol-4-yl)-8-methoxy-
2-oxo-oxazolo[5,4-c]quinolin-1-yl]ethyl]benzonitrile,
replacing 4-formylbenzonitrile with 2-formylbenzonitrile.

| Example | Structure | Name |
|---|---|---|
| 91 | 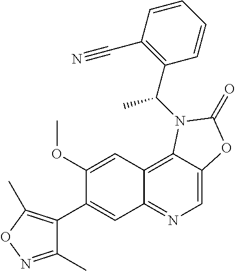 [SMILES: COc1cc2c(cc1-c1c(C)noc1C)ncc1oc(=O)n([C@H](C)c3ccccc3C#N)c12] | 2-[(1R)-1-[7-(3,5- Dimethylisoxazol- 4-yl)-8-methoxy-2- oxo-oxazolo[5,4- c]quinolin-1- yl]ethyl]benzonitrile |

TABLE 7
Examples 92-97 can be synthesized as outlined in Example 55 for 7-(3,5-dimethylisoxazol-4-yl)-
8-methoxy-1-[1-(4-pyridyl)ethyl]oxazolo[5,4-c]quinolin-2-one, replacing 1-(4-pyridyl)ethanol
with the appropriate alcohol, and replacing 7-(3,5-dimethylisoxazol-4-yl)-8-methoxy-1H-oxazolo
[5,4-c]quinolin-2-one with ethyl 7-(3,5-dimethylisoxazol-4-yl)-8-methoxy-2-oxo-1H-oxazolo
[5,4-c]quinoline-4-carboxylate, which can be synthesized as outlined in Example 11 for 7-(3,5-
dimethylisoxazol-4-yl)-8-methoxy-1H-oxazolo[5,4-c]quinolin-2-one, replacing paraformalde-
hyde with ethyl glyoxylate. The product can then be reduced as outlined in Example 35 for 7-
(3,5-dimethylisoxazol-4-yl)-4-(hydroxymethyl)-8-methoxy-1-[(1R)-1-phenylethyl]oxazolo
[5,4-c]quinolin-2-one, replacing ethyl 7-(3,5-dimethylisoxazol-4-yl)-8-methoxy-2-oxo-1-
[(1R)-1-phenylethyl]oxazolo[5,4-c]quinoline-4-carboxylate with the appropriate ester.

| Example | Structure | Name |
|---|---|---|
| 92 | 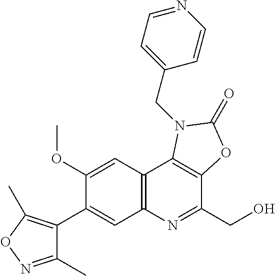 [SMILES: COc1cc2c(cc1-c1c(C)noc1C)nc(CO)c1oc(=O)n(Cc3ccncc3)c12] | 7-(3,5-Dimethylisoxazol-4-yl)-4- (hydroxymethyl)-8-methoxy-1-(4- pyridylmethyl)oxazolo[5,4- c]quinolin-2-one |
| 93 | 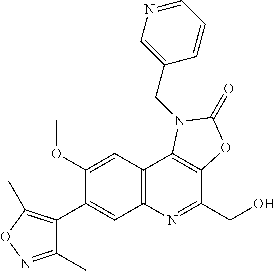 [SMILES: COc1cc2c(cc1-c1c(C)noc1C)nc(CO)c1oc(=O)n(Cc3cccnc3)c12] | 7-(3,5-Dimethylisoxazol-4-yl)-4- (hydroxymethyl)-8-methoxy-1-(3- pyridylmethyl)oxazolo[5,4- c]quinolin-2-one |
| 94 | 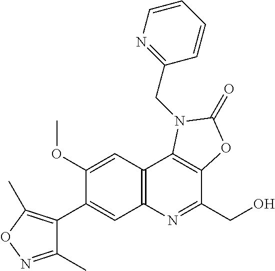 [SMILES: COc1cc2c(cc1-c1c(C)noc1C)nc(CO)c1oc(=O)n(Cc3ccccn3)c12] | 7-(3,5-Dimethylisoxazol-4-yl)-4- (hydroxymethyl)-8-methoxy-1-(2- pyridylmethyl)oxazolo[5,4- c]quinolin-2-one |
| 95 | 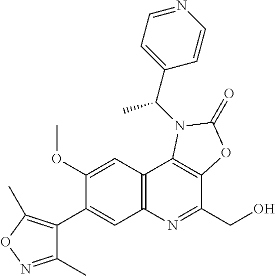 [SMILES: COc1cc2c(cc1-c1c(C)noc1C)nc(CO)c1oc(=O)n([C@H](C)c3ccncc3)c12] | 7-(3,5-Dimethylisoxazol-4-yl)-4- (hydroxymethyl)-8-methoxy-1- [(1R)-1-(4- pyridyl)ethyl]oxazolo[5,4- c]quinolin-2-one |
| 96 | 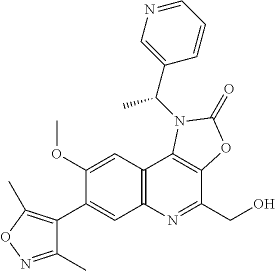 [SMILES: COc1cc2c(cc1-c1c(C)noc1C)nc(CO)c1oc(=O)n([C@H](C)c3cccnc3)c12] | 7-(3,5-Dimethylisoxazol-4-yl)-4- (hydroxymethyl)-8-methoxy-1- [(1R)-1-(3- pyridyl)ethyl]oxazolo[5,4- c]quinolin-2-one |
| 97 | 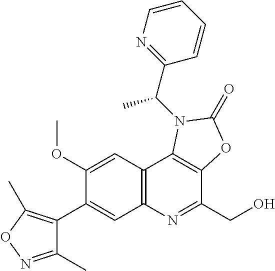 [SMILES: COc1cc2c(cc1-c1c(C)noc1C)nc(CO)c1oc(=O)n([C@H](C)c3ccccn3)c12] | 7-(3,5-Dimethylisoxazol-4-yl)-4- (hydroxymethyl)-8-methoxy-1- [(1R)-1-(2- pyridyl)ethyl]oxazolo[5,4- c]quinolin-2-one |

### Example 98

### In Vitro Bromodomain Inhibition Assay

To measure activity of bromodomain inhibitors, a His-epitope tagged BRD4 BD149-170 was purchased from BPS Bioscience. BRD4 binding and inhibition was assessed by monitoring the engagement of biotinylated H4-tetraacetyl peptide (H4K5/8/12/16; AnaSpec #64989-025) with the target using the AlphaLISA technology (Perkin-Elmer). Specifically, in a 384 well OptiPlate, BRD4(BD1) (200 nM final) was pre-incubated with either DMSO (final 1.0% DMSO) or a compound dilution series in DMSO. All reagents were diluted in assay buffer containing 50 mM HEPES (pH 7.4), 100 mM NaCl, 0.1% (w/v) BSA, and 0.05% (w/v) CHAPS. After a 30 minute incubation at room temperature, H4 peptide was added (200 nM final) and the reaction was incubated an additional 30 minutes at room temperature. Alpha. streptavidin donor beads and AlphaLISA nickel chelate acceptor beads were then added to a final concentration of 10 ug/mL each. After one hour, equilibration plates were read on an Envision instrument and IC₅₀s calculated using a four parameter non-linear curve fit. The BRD4 IC₅₀ values for the following examples were each less than 1 μM: 1, 2, 3, 12A, 18, 33, 34, 35, 36, 37, 38, 39, 40, 41, 42, 43, 44, 45, 46, 47, 48, 49, 50, 51, 52, 53, and 54.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

1.-65. (canceled)

66. A compound or compounds of Formula (I): 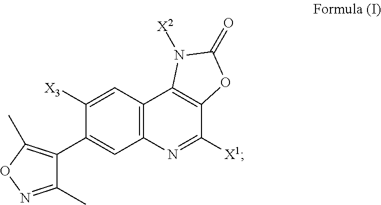
or a pharmaceutically acceptable salt thereof,
wherein:
X¹ is H, —C(O)NR¹R², —C(O)—R¹, —C(O)OR¹, cycloalkyl, heterocycloalkyl, aryl, heteroaryl, —CH₂OR¹, —CH₂R¹, or —C≡N;
X² is H, optionally substituted alkyl, optionally substituted alkenyl, optionally substituted cycloalkyl, optionally substituted aryl, optionally substituted heterocycloalkyl, optionally substituted heteroaryl, optionally substituted —CH₂-cyloalkyl, optionally substituted —CH₂-aryl, optionally substituted —CH₂-heterocycloalkyl, optionally substituted —CH₂-heteroaryl, optionally substituted —CH(C₁-C₆-alkyl)-alkyl, optionally substituted —CH(C₁-C₆-alkyl)-cycloalkyl, optionally substituted —CH(C₁-C₆-alkyl)-aryl, optionally substituted —CH(C₁-C₆-alkyl)-heterocycloalkyl, or optionally substituted —CH(C₁-C₆-alkyl)-heteroaryl;
X³ is —OR³, —C≡N, —CH₂OR³, —NH-alkyl, —N(alkyl)₂, —CH₂N(alkyl)₂, —CH₂NH(alkyl), or halogen, and
R¹, R² and R³ are each independently H, C₁-C₁₂alkyl, cycloalkyl, heterocycloalkyl, aryl, or heteroaryl optionally substituted with alkyl.

67. The compound of claim 0, wherein X³ is —OR³ and R³ is C₁-C₆alkyl.

68. The compound of claim 67, wherein X³ is —OCH₃.

69. The compound of claim 0, wherein X¹ is:
a) H;
b) —C(O)NR¹R², —C(O)R¹, —C(O)OR¹, cycloalkyl, heterocycloalkyl, aryl, heteroaryl, —CH₂OR¹, or —CH₂R¹;
c) —C(O)NR¹R², and R¹ and R² are each independently H or C₁-C₆alkyl;
d) —C(O)R¹, and R¹ is heterocycloalkyl;
e) —C(O)OR¹, and R¹ is H or C₁-C₆alkyl;
f) cycloalkyl;
g) heterocycloalkyl;
h) aryl;
i) heteroaryl;
j) —CH₂OR¹ and R¹ is aryl or C₁-C₆alkyl; or
k) —CH₂R¹ and R¹ is aryl or C₁-C₆alkyl.

70. The compound of claim 68, wherein X¹ is:
a) H;
b) —C(O)NR¹R², —C(O)R¹, —C(O)OR¹, cycloalkyl, heterocycloalkyl, aryl, heteroaryl, —CH₂OR¹, or —CH₂R¹;
c) —C(O)NR¹R², and R¹ and R² are each independently H or C₁-C₆alkyl;
d) —C(O)R¹, and R¹ is heterocycloalkyl;
e) —C(O)OR¹, and R¹ is H or C₁-C₆alkyl;
f) cycloalkyl;
g) heterocycloalkyl;
h) aryl;
i) heteroaryl;
j) —CH₂OR¹ and R¹ is aryl or C₁-C₆alkyl; or
k) —CH₂R¹ and R¹ is aryl or C₁-C₆alkyl.

71. The compound of claim 0, wherein X² is:
a) H;
b) alkenyl;
c) —CH₂-aryl optionally substituted with halogen or methoxy;
d) —CH₂-aryl optionally substituted with chloro or methoxy;
e) —CH₂-aryl;
f) —CH₂-heteroaryl optionally substituted with halogen, trifluoromethyl, or methoxy;
g) —CH₂-pyridinyl or —CH₂-furanyl;
h) unsubstituted —CH₂-pyridinyl;
i) —CH₂-heterocycloalkyl;
j) unsubstituted —CH₂-piperidinyl or unsubstituted —CH₂-tetrahydropyranyl;
k) —CH₂-cycloalkyl;
l) —CH₂-cyclohexyl;
m) —CH(C₁-C₆-alkyl)-aryl;
n) —CH(CH₃)-phenyl;
o) phenyl; or
p) pyridinyl.

72. The compound of claim 68, wherein X² is:
a) H;
b) alkenyl;
c) —CH₂-aryl optionally substituted with halogen or methoxy;
d) —CH₂-aryl optionally substituted with chloro or methoxy;
e) —CH₂-aryl;
f) —CH₂-heteroaryl optionally substituted with halogen, trifluoromethyl, or methoxy;
g) —CH₂-pyridinyl or —CH₂-furanyl;
h) unsubstituted —CH₂-pyridinyl;
i) —CH₂-heterocycloalkyl;
j) unsubstituted —CH₂-piperidinyl or unsubstituted —CH₂-tetrahydropyranyl;
k) —CH₂-cycloalkyl;
l) —CH₂-cyclohexyl;
m) —CH(C₁-C₆-alkyl)-aryl;
n) —CH(CH₃)-phenyl;
o) phenyl; or
p) pyridinyl.

73. The compound of claim 0, wherein X¹ is H, X² is —CH(CH₃)-phenyl and X³ is —OCH₃.

74. The compound of claim 0, wherein X¹ is CH₂R¹ and R¹ is C₁-C₆alkyl, X² is —CH(CH₃)-phenyl and X³ is —OCH₃.

75. The compound of claim 0, wherein X¹ is —CH₂OR¹ and R¹ is H or C₁-C₆alkyl, X² is —CH(CH₃)-phenyl and X³ is —OCH₃.

76. The compound according to claim 0, wherein the compound is selected from:
Ethyl 1-benzyl-7-(3,5-dimethylisoxazol-4-yl)-8-methoxy-2-oxo-oxazolo[5,4-c]quinoline-4-carboxylate,
1-Benzyl-7-(3,5-dimethylisoxazol-4-yl)-8-methoxy-oxazolo[5,4-c]quinolin-2-one,
7-(3,5-Dimethylisoxazol-4-yl)-8-methoxy-1-[(1R)-1-(2-pyridyl)ethyl]oxazolo[5,4-c]quinolin-2-one,
7-(3,5-Dimethylisoxazol-4-yl)-8-methoxy-1-phenyl-oxazolo[5,4-c]quinolin-2-one,
7-(3,5-Dimethylisoxazol-4-yl)-8-methoxy-1-(2-pyridyl)oxazolo[5,4-c]quinolin-2-one,
1-(Cyclohexylmethyl)-7-(3,5-dimethylisoxazol-4-yl)-8-methoxy-oxazolo[5,4-c]quinolin-2-one,
7-(3,5-Dimethylisoxazol-4-yl)-8-methoxy-1-tetrahydropyran-3-yl-oxazolo[5,4-c]quinolin-2-one,
7-(3,5-Dimethylisoxazol-4-yl)-8-methoxy-1-(tetrahydropyran-2-ylmethyl)oxazolo[5,4-c]quinolin-2-one,
7-(3,5-Dimethylisoxazol-4-yl)-8-methoxy-1-(2-piperidylmethyl)oxazolo[5,4-c]quinolin-2-one,
1-allyl-7-(3,5-dimethylisoxazol-4-yl)-8-methoxy-oxazolo[5,4-c]quinolin-2-one,
7-(3,5-dimethylisoxazol-4-yl)-8-methoxy-1H-oxazolo[5,4-c]quinolin-2-one,
7-(3,5-Dimethylisoxazol-4-yl)-8-methoxy-1-(1-phenylethyl)oxazolo[5,4-c]quinolin-2-one,
7-(3,5-Dimethylisoxazol-4-yl)-8-methoxy-1-(3-pyridylmethyl)oxazolo[5,4-c]quinolin-2-one,
7-(3,5-dimethylisoxazol-4-yl)-8-methoxy-1-[[5-(trifluoromethyl)-2-furyl]methyl]oxazolo[5,4-c]quinolin-2-one,
7-(3,5-dimethylisoxazol-4-yl)-8-methoxy-1-(4-pyridylmethyl)oxazolo[5,4-c]quinolin-2-one,
7-(3,5-dimethylisoxazol-4-yl)-8-methoxy-1-(2-pyridylmethyl)oxazolo[5,4-c]quinolin-2-one,
1-[(3-chlorophenyl)methyl]-7-(3,5-dimethylisoxazol-4-yl)-8-methoxy-oxazolo[5,4-c]quinolin-2-one,
1-[(2-chlorophenyl)methyl]-7-(3,5-dimethylisoxazol-4-yl)-8-methoxy-oxazolo[5,4-c]quinolin-2-one,
7-(3,5-dimethylisoxazol-4-yl)-8-methoxy-1-[(3-methoxyphenyl)methyl]oxazolo[5,4-c]quinolin-2-one,
7-(3,5-dimethylisoxazol-4-yl)-8-methoxy-1-[(2-methoxyphenyl)methyl]oxazolo[5,4-c]quinolin-2-one,
1-Benzyl-7-(3,5-dimethylisoxazol-4-yl)-8-methoxy-2-oxo-oxazolo[5,4-c]quinoline-4-carboxylic acid,
1-Benzyl-7-(3,5-dimethylisoxazol-4-yl)-8-methoxy-N-methyl-2-oxo-oxazolo[5,4-c]quinoline-4-carboxamide,
1-benzyl-7-(3,5-dimethylisoxazol-4-yl)-8-methoxy-N,N-dimethyl-2-oxo-oxazolo[5,4-c]quinoline-4-carboxamide,
1-Benzyl-7-(3,5-dimethylisoxazol-4-yl)-8-methoxy-4-(morpholine-4-carbonyl)oxazolo[5,4-c]quinolin-2-one,
1-Benzyl-4-cyclohexyl-7-(3,5-dimethylisoxazol-4-yl)-8-methoxy-oxazolo[5,4-c]quinolin-2-one,
1-Benzyl-7-(3,5-dimethylisoxazol-4-yl)-8-methoxy-4-(4-piperidyl)oxazolo[5,4-c]quinolin-2-one,
1-benzyl-7-(3,5-dimethylisoxazol-4-yl)-8-methoxy-4-(methoxymethyl)oxazolo[5,4-c]quinolin-2-one,
1-Benzyl-7-(3,5-dimethylisoxazol-4-yl)-8-methoxy-4-(phenoxymethyl)oxazolo[5,4-c]quinolin-2-one,
1-Benzyl-7-(3,5-dimethylisoxazol-4-yl)-8-methoxy-4-phenyl-oxazolo[5,4-c]quinolin-2-one,
1-Benzyl-7-(3,5-dimethylisoxazol-4-yl)-4-(1H-imidazol-4-yl)-8-methoxy-oxazolo[5,4-c]quinolin-2-one,
1-Benzyl-7-(3,5-dimethylisoxazol-4-yl)-4-isobutyl-8-methoxy-oxazolo[5,4-c]quinolin-2-one, and
1,4-Dibenzyl-7-(3,5-dimethylisoxazol-4-yl)-8-methoxy-oxazolo[5,4-c]quinolin-2-one; or a pharmaceutically acceptable salt thereof.

77. The compound according to claim 0 together with a pharmaceutically acceptable carrier, diluent and excipient.

78. A method of treating a disease or condition for which a bromodomain inhibitor is indicated in a subject in need thereof which comprises administering a therapeutically effective amount of a compound according to claim 0.

79. The method of claim 78 which comprises administering a therapeutically effective amount of a compound according to claim 76.

80. The method according to claim 78, wherein the disease or condition is an auto-immune disorder, an inflammatory disorder, a dermal disorder or cancer.

81. The method according to claim 78, wherein the inflammatory disorder is rheumatoid arthritis, irritable bowel syndrome, or psoriasis.

82. The method according to claim 78, wherein the disease or condition is brain cancer, pancreatic cancer, breast cancer, lung cancer, or prostate cancer.

83. The method according to claim 79, wherein the disease or condition is brain cancer, pancreatic cancer, breast cancer, lung cancer, or prostate cancer.

84. The method according to claim 82, wherein the disease or condition is glioblastoma multiforme.

85. A method for inhibiting a bromodomain which comprising contacting the bromodomain with a compound of according to claim 0.
